(19) United States
(12) Patent Application Publication
(10) Pub. No.: US 2023/0103980 A1
(43) Pub. Date: Apr. 6, 2023

(54) THERMAL STATE SWITCHES IN MACROPHAGES

(71) Applicant: California Institute of Technology, Pasadena, CA (US)

(72) Inventors: Abdullah S. Farooq, Pasadena, CA (US); Mohamad H. Abedi, Pasadena, CA (US); Mikhail G. Shapiro, Pasadena, CA (US); Ann Liu, Pasadena, CA (US)

(21) Appl. No.: 17/937,614

(22) Filed: Oct. 3, 2022

Related U.S. Application Data
(60) Provisional application No. 63/251,847, filed on Oct. 4, 2021.

(51) Int. Cl. C12N 15/82; G01N 33/53; B01L 7/00; C12N 15/63

(52) U.S. Cl. CPC C12N 15/8217; G01N 33/53; B01L 7/52; C12N 15/63

(57) ABSTRACT

Disclosed herein include methods, compositions, and kits suitable for use in spatiotemporal regulation of therapeutic macrophages through a combination of molecular and physical actuation. There are provided, in some embodiments, thermal bioswitches that allow macrophages to sense small changes in temperature and use them as inputs for the actuation of genetic circuits. Genetic circuits capable of inducing expression of a payload upon thermal stimulation are provided. There are provided, in some embodiments, heat-inducible macrophages and methods of using are provided.

### RELATED APPLICATIONS

This application claims the benefit under 35 U.S.C. § 119(e) of U.S. Provisional Patent Application Ser. No. 63/251,847, filed Oct. 4, 2021, the content of this related application is incorporated herein by reference in its entirety for all purposes.

### STATEMENT REGARDING FEDERALLY SPONSORED R&D

This invention was made with government support under Grant No. MH117080 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 30KJ-302453-US, created Oct. 1, 2022, which is 20.0 kilobytes in size. The information in the electronic format of the Sequence Listing is incorporated herein by reference in its entirety.

### BACKGROUND

### Field

The present disclosure relates generally to the field of macrophage therapies and more specifically to spatiotemporal control of macrophage activity.

### Description of the Related Art

CAR-T cell therapy has shown tremendous clinical promise in the treatment of hematologic malignancies but has struggled in the translation to solid tumours. This lack of efficacy in solid tumors is due in part to the immunosuppressive tumor microenvironment, preventing immune cells that have trafficked into the tumour from mounting an antitumor response. A critical component of the immunosuppressive tumor microenvironment is the presence of tumor-associated macrophages (TAMs). These cells modulate the immune system by secreting chemokines, cytokines, and growth factors, in addition to activating checkpoint pathways in T cells. Due to their critical role in preventing immune surveillance and activation within the tumor, TAMs are recruited in high numbers to the tumor, and can make up nearly 50% of tumor mass in certain tumors. While the recruitment of TAMs presents a unique clinical challenge to the treatment of solid tumors, this process can be capitalized upon to create more effective immunotherapies. Macrophages can be engineered to secrete molecular therapeutic payloads that manually engage the immune system, such as IL-12, a pro-inflammatory cytokine. These cells can then function as “Trojan horses” that are recruited actively to tumours and then release immunomodulators that they are engineered to express within the tumour. However, many of the benefits that molecular immunomodulators carry must be balanced with the significant risk of adverse effects if delivered in parts of the body other than the tumor, due to widespread and inappropriate immune activation. These potential effects include the development of type 1 diabetes mellitus, hypothyroidism, adrenocortical hormone insufficiency, and other autoimmune-like syndromes. These side effects have limited the clinical applicability of these immunomodulators. Since macrophages traffic to multiple physiological niches upon injection, such as the lungs and liver, it is necessary to spatially restrict the release of therapeutic payloads from macrophage-based agents to the tumour microenvironment. Systemically administered chemical inducers have been used to control immune cells in vivo, but this control modality is inherently limited by the biodistribution of the inducer molecule. Optical methods for controlling immune cells provide a high degree of spatiotemporal control, but are limited for usage in vivo due to the limited penetration of light at depth within tissues. Due to the limitations of chemical and optical control methods, there is a need for compositions and methods wherein spatiotemporal targeting is combined with the ability to penetrate deep into tissues.

### SUMMARY

Disclosed herein include populations of heat-inducible macrophages. In some embodiments, one or more of said heat-inducible macrophages comprise: a first inducible promoter operably linked to a first polynucleotide comprising a recombinase gene, wherein the first inducible promoter is capable of inducing transcription of the recombinase gene to generate a recombinase transcript upon thermal stimulation, and wherein the recombinase transcript is capable of being translated to generate a recombinase; and a second promoter and a second polynucleotide comprising a payload gene, wherein, in the absence of a recombination event, the second promoter and the second polynucleotide are not operably linked, wherein the recombinase is capable of catalyzing the recombination event, and wherein the second promoter and the second polynucleotide are operably linked after the recombination event such that the second promoter is capable of inducing transcription of the payload gene to generate a payload transcript.

In some embodiments, the recombination event comprises removal of a sequence flanked by recombinase target sites or an inversion of a sequence flanked by recombinase target sites. In some embodiments, the second polynucleotide is flanked by recombinase target sites. In some embodiments, prior to the recombination event, the sequence of the payload gene is inverted relative to the promoter. A heat-inducible macrophage can comprise: at least one stop cassette situated between the second promoter and the payload gene, wherein the stop cassette comprises one or more stop sequences, and wherein the one or more stop cassettes are flanked by recombinase target sites. In some embodiments, the at least one stop cassette is configured to prevent transcription of the payload gene and/or translation of the payload transcript. In some embodiments, the one or more stop sequences comprise a polyadenylation signal, a stop codon, a frame-shifting mutation, or any combination thereof. In some embodiments, the recombinase is Cre, Dre, Flp, KD, B2, B3, λ, HK022, HP1, γ6, ParA, Tn3, Gin, ΦC31, Bxb1, R4, derivatives thereof, or any combination thereof. In some embodiments, the recombinase is a Flp recombinase and the recombinase target sites are FRT sites. In some embodiments, the recombinase is a Cre recombinase and the recombinase target sites are loxP sites.

A heat-inducible macrophage can comprise: a third polynucleotide comprising a default gene. In some embodiments, in the absence of a recombination event, the second promoter and the third polynucleotide are operably linked such that the second promoter is capable of inducing transcription of the default gene to generate a default transcript. In some embodiments, the default transcript is capable of being translated to generate a default protein. In some embodiments, the second promoter and the third polynucleotide are not operably linked after the recombination event such that the second promoter is no longer capable of inducing transcription of the default gene to generate a default transcript.

In some embodiments, default gene encodes a detectable protein, optionally the detectable protein comprises green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mScarlet, mApple, mCitrine, mCherry, mruby3, rsCherry, rsCherryRev, derivatives thereof, or any combination thereof.

In some embodiments, the second promoter comprises a ubiquitous promoter, optionally the ubiquitous promoter is selected from the group comprising a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CBH promoter, or any combination thereof.

In some embodiments, the default gene is a pro-death gene encoding a pro-death protein, optionally the pro-death protein is capable of halting cell growth and/or inducing cell death, optionally the third polynucleotide further comprises a detectable gene encoding a detectable protein, optionally the detectable protein comprises green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mScarlet, mApple, mCitrine, mCherry, mruby3, rsCherry, rsCherryRev, derivatives thereof, or any combination thereof, optionally the detectable gene and the pro-death gene are operably linked to a tandem gene expression element, optionally the tandem gene expression element is an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof.

In some embodiments, the payload protein is a pro-death protein, optionally the pro-death protein is capable of halting cell growth and/or inducing cell death, further optionally the pro-death protein comprises a secretion tag, optionally the secretion tag is selected from the group comprising AbnA, AmyE, AprE, BglC, BglS, Bpr, Csn, Epr, Ggt, GlpQ, HtrA, LipA, LytD, MntA, Mpr, NprE, OppA, PbpA, PbpX, Pel, PelB, PenP, PhoA, PhoB, PhoD, PstS, TasA, Vpr, WapA, WprA, XynA, XynD, YbdN, Ybxl, YcdH, YclQ, YdhF, YdhT, YfkN, YflE, YfmC, Yfnl, YhcR, YlqB, YncM, YnfF, YoaW, YocH, YolA, YqiX, Yqxl, YrpD, YrpE, YuaB, Yurl, YvcE, YvgO, YvpA, YwaD, YweA, YwoF, YwtD, YwtF, YxaLk, YxiA, and YxkC, optionally heat-inducible macrophages that have undergone the recombination event are capable of secreting said pro-death protein and killing bystander tumor cells.

In some embodiments, the pro-death protein comprises cytosine deaminase, iCasp9, thymidine kinase, Bax, Bid, Bad, Bak, BCL2L11, p53, PUMA, Diablo/SMAC, S-TRAIL, Cas9, Cas9n, hSpCas9, hSpCas9n, HSVtk, cholera toxin, diphtheria toxin, alpha toxin, anthrax toxin, exotoxin, pertussis toxin, Shiga toxin, shiga-like toxin Fas, TNF, caspase 2, caspase 3, caspase 6, caspase 7, caspase 8, caspase 9, caspase 10, caspase 11, caspase 12, purine nucleoside phosphorylase, or any combination thereof. In some embodiments, the pro-death protein is capable of halting cell growth and/or inducing cell death in the presence of a pro-death agent. In some embodiments, pro-death protein comprises iCasp9 and the pro-death agent comprises AP20187. In some embodiments, the pro-death protein comprises Caspase-9 and the pro-death agent comprises AP1903. In some embodiments, the pro-death protein comprises HSV thymidine kinase (TK) and the pro-death agent comprises Ganciclovir (GCV), Ganciclovir elaidic acid ester, Penciclovir (PCV), Acyclovir (ACV), Valacyclovir (VCV), (E)-5-(2-bromovinyl)-2′-deoxyuridine (BVDU), Zidovuline (AZT), and/or 2′-exo-methanocarbathymidine (MCT). In some embodiments, the pro-death protein comprises Cytosine Deaminase (CD) and the pro-death agent comprises 5-fluorocytosine (5-FC). In some embodiments, the pro-death protein comprises Purine nucleoside phosphorylase (PNP) and the pro-death agent comprises 6-methylpurine deoxyriboside (MEP) and/or fludarabine (FAMP). In some embodiments, the pro-death protein comprises a Cytochrome p450 enzyme (CYP) and the pro-death agent comprises Cyclophosphamide (CPA), Ifosfamide (IFO), and/or 4-ipomeanol (4-IM). In some embodiments, the pro-death protein comprises a Carboxypeptidase (CP) and the pro-death agent comprises 4-[(2-chloroethyl)(2-mesyloxyethyl)amino]benzoyl-L-glutamic acid (CMDA), Hydroxy-and amino-aniline mustards, Anthracycline glutamates, and/or Methotrexate α-peptides (MTX-Phe). In some embodiments, the pro-death protein comprises Carboxylesterase (CE) and the pro-death agent comprises Irinotecan (IRT), and/or Anthracycline acetals. In some embodiments, the pro-death protein comprises Nitroreductase (NTR) and the pro-death agent comprises dinitroaziridinylbenzamide CB1954, dinitrobenzamide mustard SN23862, 4-Nitrobenzyl carbamates, and/or Quinones. In some embodiments, the pro-death protein comprises Horse radish peroxidase (HRP) and the pro-death agent comprises Indole-3-acetic acid (IAA) and/or 5-Fluoroindole-3-acetic acid (FIAA). In some embodiments, the pro-death protein comprises Guanine Ribosyltransferase (XGRTP) and the pro-death agent comprises 6-Thioxanthine (6-TX); the pro-death protein comprises a glycosidase enzyme and the pro-death agent comprises HM1826 and/or Anthracycline acetals. In some embodiments, the pro-death protein comprises Methionine-α,γ-lyase (MET) and the pro-death agent comprises Selenomethionine (SeMET). In some embodiments, the pro-death protein comprises thymidine phosphorylase (TP) and the pro-death agent comprises 5′-Deoxy-5-fluorouridine (5′-DFU).

Disclosed herein include populations of heat-inducible macrophages. In some embodiments, one or more of said heat-inducible macrophages comprise: a first inducible promoter operably linked to a first polynucleotide comprising a transactivator gene, and a second promoter operably linked to a second polynucleotide comprising a payload gene, wherein the first inducible promoter is capable of inducing transcription of the transactivator gene to generate a transactivator transcript in the presence of thermal stimulation, wherein the transactivator transcript is capable of being translated to generate a transactivator; and wherein, in the presence of the transactivator and a transactivator-binding compound, the second promoter is capable of inducing transcription of the payload gene to generate a payload transcript.

Disclosed herein include populations of heat-inducible macrophages. In some embodiments, one or more of said heat-inducible macrophages comprise: a first inducible promoter and a second promoter each operably linked to a first polynucleotide comprising a payload gene and to a second polynucleotide comprising a transactivator gene, wherein the first inducible promoter is capable of inducing transcription of the payload gene and the transactivator gene to generate a polycistronic transcript upon thermal stimulation, wherein, in the presence of the transactivator and a transactivator-binding compound, the second promoter is capable of inducing transcription of the payload gene and the transactivator gene to generate a polycistronic transcript, and wherein the polycistronic transcript is capable of being translated to generate the transactivator and a payload protein and/or payload RNA agent.

In some embodiments, the second promoter comprises one or more copies of a transactivator recognition sequence the transactivator is capable of binding to induce transcription, and wherein the transactivator is incapable of binding the transactivator recognition sequence in the absence of the transactivator-binding compound, optionally the one or more copies of a transactivator recognition sequence comprise one or more copies of a tet operator (TetO).

Disclosed herein include populations of heat-inducible macrophages. In some embodiments, one or more of said heat-inducible macrophages comprise: a first inducible promoter operably linked to a first polynucleotide comprising a transactivator gene, and a second promoter operably linked to a second polynucleotide comprising a payload gene, wherein the first inducible promoter is capable of inducing transcription of the transactivator gene to generate a transactivator transcript in the presence of thermal stimulation, wherein the second promoter comprises one or more copies of a transactivator recognition sequence the transactivator is capable of binding to induce transcription, and wherein the transactivator is incapable of binding the transactivator recognition sequence in the presence of the transactivator-binding compound, wherein the transactivator transcript is capable of being translated to generate a transactivator; and wherein, in the presence of the transactivator and in the absence of transactivator-binding compound, the second promoter is capable of inducing transcription of the payload gene to generate a payload transcript.

Disclosed herein include populations of heat-inducible macrophages. In some embodiments, one or more of said heat-inducible macrophages comprise: a first inducible promoter and a second promoter each operably linked to a first polynucleotide comprising a payload gene and to a second polynucleotide comprising a transactivator gene, wherein the first inducible promoter is capable of inducing transcription of the payload gene and the transactivator gene to generate a polycistronic transcript upon thermal stimulation, wherein the second promoter comprises one or more copies of a transactivator recognition sequence the transactivator is capable of binding to induce transcription, and wherein the transactivator is incapable of binding the transactivator recognition sequence in the presence of the transactivator-binding compound, wherein, in the presence of the transactivator and in the absence of a transactivator-binding compound, the second promoter is capable of inducing transcription of the payload gene and the transactivator gene to generate a polycistronic transcript, and wherein the polycistronic transcript is capable of being translated to generate the transactivator and a payload protein and/or payload RNA agent.

In some embodiments, the one or more copies of a transactivator recognition sequence comprise one or more copies of a tet operator (TetO). In some embodiments, the second promoter comprises a tetracycline response element (TRE), and wherein the TRE comprises one or more copies of a tet operator (TetO). In some embodiments, the transactivator comprises reverse tetracycline-controlled transactivator (rtTA). In some embodiments, the transactivator comprises tetracycline-controlled transactivator (tTA). In some embodiments, the transactivator-binding compound comprises tetracycline, doxycycline or a derivative thereof. In some embodiments, the first polynucleotide and the second polynucleotide are operably linked to a tandem gene expression element, optionally the tandem gene expression element is an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof. In some embodiments, the payload protein and the transactivator are expressed as separate proteins.

Disclosed herein include populations of heat-inducible macrophages. In some embodiments, one or more of said heat-inducible macrophages comprise: a first inducible promoter operably linked to a first polynucleotide comprising a payload gene, wherein the first inducible promoter is capable of inducing transcription of the payload gene to generate a payload transcript upon thermal stimulation.

In some embodiments, the payload transcript is capable of being translated to generate a payload protein. In some embodiments, thermal stimulation comprises heating to an activating temperature. In some embodiments, the activating temperature is about 37.5° C., about 38.0° C., about 38.5° C., about 39.0° C., about 39.5° C., about 40.0° C., about 40.5° C., about 41.0° C., about 41.5° C., about 42.0° C., about 42.5° C., about 43.0° C., about 43.5° C., about 44.0° C., about 44.5° C., about 45.0° C., about 45.5° C., or about 46.0° C.

In some embodiments, in the absence of thermal stimulation, the payload protein reaches unstimulated steady state payload protein levels in the heat-inducible macrophage, optionally unstimulated steady state payload protein levels are insufficient to exert a phenotypic effect and/or therapeutic effect on said heat-inducible macrophage. In some embodiments, upon thermal stimulation, transcription of the payload gene, transactivator gene, and/or recombinase gene from the first inducible promoter is increased by at least 1.1-fold. In some embodiments, the steady-state levels of the payload transcript, the steady-state levels of transactivator transcript, the steady-state levels of recombinase transcript, and/or the steady-state levels of the polycistronic transcript are at least 1.1 higher upon thermal stimulation. In some embodiments, upon thermal stimulation, the payload protein reaches stimulated steady state payload protein levels in the heat-inducible macrophage, optionally: the payload protein does not return to unstimulated steady state payload protein levels; or the payload protein does not return to unstimulated steady state payload protein levels for at least about 4 weeks, about 3 weeks, about 2 weeks, about 1 week, about 6 days, about 5 days, about 96 hours, about 48 hours, about 44 hours, about 40 hours, about 35 hours, about 30 hours, about 25 hours, 20 hours, 15 hours, 10 hours, about 8 hours, about 8 hours, 8 hours, about 7 hours, about 6 hours, about 5 hours, about 4 hours, about 3 hours, about 2 hours, about 1 hour, about 30 minutes, about 15 minutes, about 10 minutes, or about 5 minutes. In some embodiments, stimulated steady state payload protein levels are at least 1.1-fold higher than unstimulated steady state payload protein levels. In some embodiments, increasing transactivator-binding compound concentration increases stimulated steady state payload protein levels.

In some embodiments, the first inducible promoter comprises or is derived from a mammalian heat shock promoter (HSP) or a C. elegans HSP, optionally the mammalian HSP is a human HSP or mouse HSP. In some embodiments, the first inducible promoter comprises a nucleotide sequence that is at least 80%, 85%, 90%, 95%, 98%, 99%, or 100% identical to any one of SEQ ID NOS: 1-14. In some embodiments, the first inducible promoter comprises one or more heat shock element (HSE) binding sites, optionally four HSE binding sites. In some embodiments, the first inducible promoter does not comprise a human transcription factor binding site other than one or more HSE binding sites. In some embodiments, the first polynucleotide and/or second polynucleotide comprises one or more transcript stabilization element(s). In some embodiments, the transcript stabilization element comprises woodchuck hepatitis post-translational regulatory element (WPRE), bovine growth hormone polyadenylation (bGH-polyA) signal sequence, human growth hormone polyadenylation (hGH-polyA) signal sequence, or any combination thereof.

In some embodiments, the payload RNA agent comprises one or more of dsRNA, siRNA, shRNA, pre-miRNA, pri-miRNA, miRNA, stRNA, lncRNA, piRNA, and snoRNA. In some embodiments, the first polynucleotide or the second polynucleotide comprises a payload gene and one or more secondary payload gene(s). In some embodiments, said secondary payload gene(s) encode one or more secondary payload RNA agent(s) and/or one secondary payload protein(s). In some embodiments, the payload gene and the one or more secondary payload gene(s) are operably linked to a tandem gene expression element, optionally the tandem gene expression element is an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof. In some embodiments, the payload transcript or the polycistronic transcript is capable of being translated to generate: (i) a payload protein or payload RNA agent, and (ii) one or more secondary payload RNA agents and/or one or more secondary payload proteins. In some embodiments, a payload protein is capable of diminishing the concentration, stability, and/or activity an endogenous protein. In some embodiments, a payload protein comprises a component of a synthetic protein circuit. In some embodiments, the payload protein is a therapeutic protein or a variant thereof, optionally a therapeutic protein configured to prevent or treat a disease or disorder of a subject, further optionally the subject suffers from a deficiency of said therapeutic protein.

In some embodiments, a payload protein comprises fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof. In some embodiments, a payload protein comprises nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof. In some embodiments, a payload protein comprises a CRE recombinase, GCaMP, a cell therapy component, a knock-down gene therapy component, a cell-surface exposed epitope, or any combination thereof. In some embodiments, a payload protein comprises a diagnostic agent, optionally wherein the diagnostic agent comprises green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCitrine, mCherry, mruby3 rsCherry, rsCherryRev, derivatives thereof, or any combination thereof.

In some embodiments, a payload protein comprises a bispecific T cell engager (BiTE), optionally obinutuzumab, mosunetuzumab, selicrelumab, blinatumomab, ertumaxomab, maxomab, AMV564, AFM13, REGN-1979, GEN-3013, or pasotuxizumab. In some embodiments, a payload protein comprises a cytokine, optionally the cytokine is selected from the group consisting of interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, granulocyte macrophage colony stimulating factor (GM-CSF), M-CSF, SCF, TSLP, oncostatin M, leukemia-inhibitory factor (LIF), CNTF, Cardiotropin-1, NNT-1/BSF-3, growth hormone, Prolactin, Erythropoietin, Thrombopoietin, Leptin, G-CSF, or receptor or ligand thereof. In some embodiments, a payload protein comprises a member of the TGF-β/BMP family selected from the group consisting of TGF-β1, TGF-β2, TGF-β3, BMP-2, BMP-3a, BMP-3b, BMP-4, BMP-5, BMP-6, BMP-7, BMP-8a, BMP-8b, BMP-9, BMP-10, BMP-11, BMP-15, BMP-16, endometrial bleeding associated factor (EBAF), growth differentiation factor-1 (GDF-1), GDF-2, GDF-3, GDF-5, GDF-6, GDF-7, GDF-8, GDF-9, GDF-12, GDF-14, mullerian inhibiting substance (MIS), activin-1, activin-2, activin-3, activin-4, and activin-5. In some embodiments, a payload protein comprises a member of the TNF family of cytokines selected from the group consisting of TNF-alpha, TNF-beta, LT-beta, CD40 ligand, Fas ligand, CD 27 ligand, CD 30 ligand, and 4-1 BBL.

In some embodiments, a payload protein comprises a member of the immunoglobulin superfamily of cytokines selected from the group consisting of B7.1 (CD80) and B7.2 (B70). In some embodiments, a payload protein comprises an interferon, optionally the interferon is selected from interferon alpha, interferon beta, or interferon gamma. In some embodiments, a payload protein comprises a chemokine, optionally the chemokine is selected from CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13, or CXCL15. In some embodiments, a payload protein comprises an interleukin, optionally the interleukin is selected from IL-10 IL-12, IL-1, IL-6, IL-7, IL-15, IL-2, IL-18 or IL-21. In some embodiments, a payload protein comprises a tumor necrosis factor (TNF), optionally the TNF is selected from TNF-alpha, TNF-beta, TNF-gamma, CD252, CD154, CD178, CD70, CD153, or 4-1BBL. In some embodiments, a payload protein comprises a factor locally down-regulating the activity of endogenous immune cells. In some embodiments, a payload protein is capable of remodeling a tumor microenvironment and/or reducing immunosuppression at a target site of a subject.

In some embodiments, a payload protein comprises a chimeric antigen receptor (CAR) or T-cell receptor (TCR). In some embodiments, the CAR and/or TCR comprises one or more of an antigen binding domain, a transmembrane domain, and an intracellular signaling domain. In some embodiments, the intracellular signaling domain comprises a primary signaling domain, a costimulatory domain, or both of a primary signaling domain and a costimulatory domain. In some embodiments, the primary signaling domain comprises a functional signaling domain of one or more proteins selected from the group consisting of CD3 zeta, CD3 gamma, CD3 delta, CD3 epsilon, common FcR gamma (FCERIG), FcR beta (Fc Epsilon Rib), CD79a, CD79b, Fcgamma RIIa, DAP10, and DAP12, or a functional variant thereof. In some embodiments, the costimulatory domain comprises a functional domain of one or more proteins selected from the group consisting of CD27, CD28, 4-1BB (CD137), OX40, CD28-OX40, CD28-4-1BB, CD30, CD40, PD-1, ICOS, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D, or a functional variant thereof.

In some embodiments, the antigen binding domain binds a tumor antigen, optionally the tumor antigen is a solid tumor antigen. In some embodiments, the tumor antigen is selected from the group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

In some embodiments, the tumor antigen is selected from the group comprising CD150, 5T4, ActRIIA, B7, BMCA, CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRvIII, EGP-2, EGP-4, EPHa2, ErbB2, ErbB3, ErbB4, FBP, GD2, GD3, HER1-HER2 in combination, HER2-HER3 in combination, HERV-K, HIV-1 envelope glycoprotein gp120, HIV-1 envelope glycoprotein gp41, HLA-DR, HM1.24, HMW-MAA, Her2, Her2/neu, IGF-1R, IL-11Ralpha, IL-13R-alpha2, IL-2, IL-22R-alpha, IL-6, IL-6R, Ia, Ii, L1-CAM, L1-cell adhesion molecule, Lewis Y, L1-CAM, MAGE A3, MAGE-A1, MART-1, MUC1, NKG2C ligands, NKG2D Ligands, NY-ESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-1, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D1), ephrinB2, epithelial tumor antigen, estrogen receptor, fetal acethycholine e receptor, folate binding protein, gp100, hepatitis B surface antigen, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, β2-Microglobulin, Fc Receptor-like 5 (FcRL5), or molecules expressed by HIV, HCV, HBV, or other pathogens.

In some embodiments, the antigen binding domain comprises an antibody, an antibody fragment, an scFv, a Fv, a Fab, a (Fab′)2, a single domain antibody (SDAB), a VH or VL domain, a camelid VHH domain, a Fab, a Fab′, a F(ab′)2, a Fv, a scFv, a dsFv, a diabody, a triabody, a tetrabody, a multispecific antibody formed from antibody fragments, a single-domain antibody (sdAb), a single chain comprising cantiomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof. In some embodiments, the antigen binding domain is connected to the transmembrane domain by a hinge region.

In some embodiments, the transmembrane domain comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C, or a functional variant thereof. In some embodiments, the CAR or TCR further comprises a leader peptide. In some embodiments, the TCR further comprises a constant region and/or CDR4.

In some embodiments, a payload protein comprises a programmable nuclease, optionally the programmable nuclease is selected from the group comprising: SpCas9 or a derivative thereof, VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof, dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions. In some embodiments, the programmable nuclease comprises a zinc finger nuclease (ZFN) and/or transcription activator-like effector nuclease (TALEN). In some embodiments, the programmable nuclease comprises Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease, Cas1, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13c, derivatives thereof, or any combination thereof. In some embodiments, the population further comprises a polynucleotide encoding (i) a targeting molecule and/or (ii) a donor nucleic acid, optionally a payload protein comprises (i) a targeting molecule and/or (ii) a donor nucleic acid. In some embodiments, the targeting molecule is capable of associating with the programmable nuclease, optionally wherein the targeting molecule comprises single strand DNA or single strand RNA, further optionally wherein the targeting molecule comprises a single guide RNA (sgRNA).

In some embodiments, the macrophages comprise one or more receptors and/or targeting moieties configured to bind a component of a target site of a subject, optionally said one or more receptors and/or targeting moieties are payload protein(s). In some embodiments, the one or more receptors and/or the one or more targeting moieties are selected from the group comprising mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, and an RGD peptide or RGD peptide mimetic.

In some embodiments, the one or more targeting moieties and/or one or more receptors comprise one or more of the following: an antibody or antigen-binding fragment thereof, a peptide, a polypeptide, an enzyme, a peptidomimetic, a glycoprotein, a lectin, a nucleic acid, a monosaccharide, a disaccharide, a trisaccharide, an oligosaccharide, a polysaccharide, a glycosaminoglycan, a lipopolysaccharide, a lipid, a vitamin, a steroid, a hormone, a cofactor, a receptor, a receptor ligand, and analogs and derivatives thereof. In some embodiments, the antibody or antigen-binding fragment thereof comprises a Fab, a Fab′, a F(ab′)2, a Fv, a scFv, a dsFv, a diabody, a triabody, a tetrabody, a multispecific antibody formed from antibody fragments, a single-domain antibody (sdAb), a single chain comprising complementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof.

In some embodiments, the one or more targeting moieties and/or one or more receptors are configured to bind one or more of the following: CD3, CD4, CD5, CD6, CD7, CD8, CD9, CD10, CD11a, CD11b, CD11c, CD12w, CD14, CD15, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD46, CD47, CD48, CD49b, CD49c, CD51, CD52, CD53, CD54, CD55, CD56, CD58, CD59, CD61, CD62E, CD62L, CD62P, CD63, CD66, CD68, CD69, CD70, CD72, CD74, CD79, CD79a, CD79b, CD80, CD81, CD82, CD83, CD86, CD87, CD88, CD89, CD90, CD91, CD95, CD96, CD98, CD100, CD103, CD105, CD106, CD109, CD117, CD120, CD125, CD126, CD127, CD133, CD134, CD135, CD137, CD138, CD141, CD142, CD143, CD144, CD147, CD151, CD147, CD152, CD154, CD156, CD158, CD163, CD166, CD168, CD174, CD180, CD184, CDw186, CD194, CD195, CD200, CD200a, CD200b, CD209, CD221, CD227, CD235a, CD240, CD262, CD271, CD274, CD276 (B7-H3), CD303, CD304, CD309, CD326, 4-1BB, 5 AC, 5T4 (Trophoblast glycoprotein, TPBG, 5T4, Wnt-Activated Inhibitory Factor 1 or WAIF1), Adenocarcinoma antigen, AGS-5, AGS-22M6, Activin receptor like kinase 1, AFP, AKAP-4, ALK, Alpha integrin, Alpha v beta6, Amino-peptidase N, Amyloid beta, Androgen receptor, Angiopoietin 2, Angiopoietin 3, Annexin A1, Anthrax toxin protective antigen, Anti-transferrin receptor, AOC3 (VAP-1), B7-H3, Bacillus anthracis anthrax, BAFF (B-cell activating factor), B-lymphoma cell, bcr-abl, Bombesin, BORIS, C5, C242 antigen, CA125 (carbohydrate antigen 125, MUC16), CA-IX (CAIX, carbonic anhydrase 9), CALLA, CanAg, Canis lupus familiaris IL31, Carbonic anhydrase IX, Cardiac myosin, CCL11 (C-C motif chemokine 11), CCR4 (C-C chemokine receptor type 4, CD194), CCR5, CD3E (epsilon), CEA (Carcinoembryonic antigen), CEACAM3, CEACAM5 (carcinoembryonic antigen), CFD (Factor D), Ch4D5, Cholecystokinin 2 (CCK2R), CLDN18 (Claudin-18), Clumping factor A, CRIPTO, FCSF1R (Colony stimulating factor 1 receptor, CD 115), CSF2 (colony stimulating factor 2, Granulocyte-macrophage colony-stimulating factor (GM-CSF)), CTLA4 (cytotoxic T-lymphocyte-associated protein 4), CTAA16.88 tumor antigen, CXCR4 (CD 184), C-X-C chemokine receptor type 4, cyclic ADP ribose hydrolase, Cyclin B 1, CYP1B 1, Cytomegalovirus, Cytomegalovirus glycoprotein B, Dabigatran, DLL4 (delta-like-ligand 4), DPP4 (Dipeptidyl-peptidase 4), DR5 (Death receptor 5), E. coli Shiga toxin type-1, E. coli Shiga toxin type-2, ED-B, EGFL7 (EGF-like domain-containing protein 7), EGFR, EGFRII, EGFRvIII, Endoglin (CD 105), Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, FOLR (folate receptor), Folate receptor alpha, Folate hydrolase, Fos-related antigen 1.F protein of respiratory syncytial virus, Frizzled receptor, Fucosyl GM1, GD2 ganglioside, G-28 (a cell surface antigen glycolipid), GD3 idiotype, GloboH, Glypican 3, N-glycolylneuraminic acid, GM3, GMCSF receptor α-chain, Growth differentiation factor 8, GP100, GPNMB (Transmembrane glycoprotein NMB), GUCY2C (Guanylate cyclase 2C, guanylyl cyclase C (GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-γ, Influenza hemagglutinin, IgE, IgE Fc region, IGHE, IL-1, IL-2 receptor (interleukin 2 receptor), IL-4, IL-5, IL-6, IL-6R (interleukin 6 receptor), IL-9, IL-10, IL-12, IL-13, IL-17, IL-17A, IL-20, IL-22, IL-23, IL31RA, ILGF2 (Insulin-like growth factor 2), Integrins (α4, αᵤβ₃, αvβ₃, α₄β₇, α5β1, α6β4, α7β7, α11β3, α5β5, αvβ5), Interferon gamma-induced protein, ITGA2, ITGB2, KIR2D, LCK, Le, Legumain, Lewis-Y antigen, LFA-1 (Lymphocyte function-associated antigen 1, CD11a), LHRH, LINGO-1, Lipoteichoic acid, LIVIA, LMP2, LTA, MAD-CT-1, MAD-CT-2, MAGE-1, MAGE-2, MAGE-3, MAGE A1, MAGE A3, MAGE 4, MARTI, MCP-1, MIF (Macrophage migration inhibitory factor, or glycosylation inhibiting factor (GIF)), MS4A1 (membrane-spanning 4-domains subfamily A member 1), MSLN (mesothelin), MUCl (Mucin 1, cell surface associated (MUC1) or polymorphic epithelial mucin (PEM)), MUC1-KLH, MUC16 (CA125), MCPl (monocyte chemotactic protein 1), MelanA/MARTl, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES 1, P21, p53 nonmutant, P97, Page 4, PAP, Paratope of anti-(N-glycolylneuraminic acid), PAX3, PAX5, PCSK9, PDCD1 (PD-1, Programmed cell death protein 1, CD279), PDGF-Ra (Alpha-type platelet-derived growth factor receptor), PDGFR-β, PDL-1, PLAC1, PLAP-like testicular alkaline phosphatase, Platelet-derived growth factor receptor beta, Phosphate-sodium co-transporter, PMEL 17, Polysialic acid, Proteinase3 (PR1), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI), CD240), Rhesus factor, RANKL, RhoC, Ras mutant, RGS5, ROBO4, Respiratory syncytial virus, RON, Sarcoma translocation breakpoints, SART3, Sclerostin, SLAMF7 (SLAM family member 7), Selectin P, SDC1 (Syndecan 1), sLe(a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-a, TGF-β (Transforming growth factor beta), TGF-β1, TGF-β2 (Transforming growth factor-beta 2), Tie (CD202b), Tie2, TIM-1 (CDX-014), Tn, TNF, TNF-a, TNFRSF8, TNFRSF10B (tumor necrosis factor receptor superfamily member 10), TNFRSF13B (tumor necrosis factor receptor superfamily member 13B), TPBG (trophoblast glycoprotein), TRAIL-R1 (Tumor necrosis apoptosis Inducing ligand Receptor 1), TRAILR2 (Death receptor 5 (DR5)), tumor-associated calcium signal transducer 2, tumor specific glycosylation of MUC1, TWEAK receptor, TYRP1 (glycoprotein 75), TRP-2, Tyrosinase, VCAM-1 (CD 106), VEGF, VEGF-A, VEGF-2 (CD309), VEGFR-1, VEGFR2, or vimentin, WT1, XAGE 1, or cells expressing any insulin growth factor receptors, or any epidermal growth factor receptors.

In some embodiments, the macrophages do not comprise an exogenous receptor or targeting moiety configured to bind a component of a target site of a subject, optionally the macrophages are capable of trafficking to solid tumor(s) of a subject via endogenous physiological signals. In some embodiments, a payload protein comprises an agonistic or antagonistic antibody or antigen-binding fragment thereof specific to a checkpoint inhibitor or checkpoint stimulator molecule, optionally PD1, PD-L1, PD-L2, CD27, CD28, CD40, CD137, OX40, GITR, ICOS, A2AR, B7-H3, B7-H4, BTLA, CTLA4, IDO, KIR, LAG3, PD-1, and/or TIM-3. In some embodiments, the one or more payloads comprise a secretion tag, optionally the secretion tag is selected from the group comprising AbnA, AmyE, AprE, BglC, BglS, Bpr, Csn, Epr, Ggt, GlpQ, HtrA, LipA, LytD, MntA, Mpr, NprE, OppA, PbpA, PbpX, Pel, PelB, PenP, PhoA, PhoB, PhoD, PstS, TasA, Vpr, WapA, WprA, XynA, XynD, YbdN, Ybxl, YcdH, YclQ, YdhF, YdhT, YfkN, YflE, YfmC, Yfnl, YhcR, YlqB, YncM, YnfF, YoaW, YocH, YolA, YqiX, Yqxl, YrpD, YrpE, YuaB, Yurl, YvcE, YvgO, YvpA, YwaD, YweA, YwoF, YwtD, YwtF, YxaLk, YxiA, and YxkC.

In some embodiments, a payload protein comprises a constitutive signal peptide for protein degradation, optionally PEST. In some embodiments, a payload protein comprises a nuclear localization signal (NLS) or a nuclear export signal (NES). In some embodiments, a payload protein comprises a dosage indicator protein, optionally the dosage indicator protein is detectable, optionally the dosage indicator protein comprises green fluorescent protein (GFP), enhanced green fluorescent protein (EGFP), yellow fluorescent protein (YFP), enhanced yellow fluorescent protein (EYFP), blue fluorescent protein (BFP), mScarlet, red fluorescent protein (RFP), TagRFP, Dronpa, Padron, mApple, mCherry, mruby3, rsCherry, rsCherryRev, derivatives thereof, or any combination thereof. In some embodiments, a payload protein comprises a degron, optionally the steady-state levels of the payload protein can be varied by varying the sequence of the degron. In some embodiments, the macrophages are derived from blood, cord blood, bone marrow, or iPSC. In some embodiments, the macrophages are autologous macrophages and/or allogeneic macrophages. In some embodiments, the macrophages are Kupffer cells, stellate macrophages, M1 macrophages, M2 macrophages, tumor-associated macrophages (TAMs), or any combination thereof.

Disclosed herein include methods of treating a disease or disorder in a subject. In some embodiments, the method comprises: administering to the subject an effective amount of a population of heat-inducible macrophages disclosed herein. The method can comprise: applying thermal energy to a target site of the subject sufficient to increase the local temperature of the target site to an activating temperature, thereby inducing the expression of the payload in heat-inducible macrophages at the target site.

In some embodiments, the activating temperature is about 37.5° C., about 38.0° C., about 38.5° C., about 39.0° C., about 39.5° C., about 40.0° C., about 40.5° C., about 41.0° C., about 41.5° C., about 42.0° C., about 42.5° C., about 43.0° C., about 43.5° C., about 44.0° C., about 44.5° C., about 45.0° C., about 45.5° C., or about 46.0° C. In some embodiments, applying thermal energy to a target site of the subject comprises the application of one or more of focused ultrasound (FUS), magnetic hyperthermia, microwaves, infrared irradiation, liquid-based heating, and contact heating, optionally liquid-based heating comprises intraperitoneal chemotherapy (HIPEC). In some embodiments, the period of time between the administering and applying thermal energy is about 48 hours, about 44 hours, about 40 hours, about 35 hours, about 30 hours, about 25 hours, 20 hours, 15 hours, 10 hours, about 8 hours, about 8 hours, 8 hours, about 7 hours, about 6 hours, about 5 hours, about 4 hours, about 3 hours, about 2 hours, about 1 hour, about 30 minutes, about 15 minutes, about 10 minutes, or about 5 minutes. In some embodiments, applying thermal energy to a target site comprises a continuous application of thermal energy to the target site over a duration of time. In some embodiments, applying thermal energy to a target site comprises applying one or more pulses of thermal energy to the target site over a duration of time. In some embodiments, the duration of time is about 48 hours, about 44 hours, about 40 hours, about 35 hours, about 30 hours, about 25 hours, 20 hours, 15 hours, 10 hours, about 8 hours, about 8 hours, 8 hours, about 7 hours, about 6 hours, about 5 hours, about 4 hours, about 3 hours, about 2 hours, about 1 hour, about 30 minutes, about 15 minutes, about 10 minutes, or about 5 minutes. In some embodiments, the one or more pulses have a duty cycle of greater than about 1% and less than about 100%. In some embodiments, the one or more pulses each have a pulse duration of about 1 hour, about 30 minutes, about 15 minutes, about 10 minutes, or about 5 minutes, about 1 minute, about 1 second, or about 1 millisecond.

The method can comprise: monitoring the temperature of the target region, optionally the monitoring is performed by magnetic resonance imaging (MRI) and/or fiber optic thermometry. In some embodiments, the application of thermal energy to a target site of the subject is guided spatially by magnetic resonance imaging (MRI). In some embodiments, upon administration, the macrophages migrate to tumor(s) of the subject, optionally at least about 20 percent of the population of macrophages migrate to tumor(s) of the subject, further optionally said macrophages constitute at least about 10 percent of the volume of said tumor(s) about 7 days post-administration. In some embodiments, the administration of the population of macrophages does not cause neurological side effects or the development of type 1 diabetes mellitus, hypothyroidism, adrenocortical hormone insufficiency, and/or other autoimmune-like syndromes. In some embodiments, at least about 20 percent of the population of macrophages present in the target site of the subject have undergone the recombination event about four days after applying thermal energy to the target site. In some embodiments, administering to the subject an effective amount of the population of heat-inducible macrophages comprises two or more administrations. In some embodiments, applying thermal energy to a target site of the subject comprises applying thermal energy to two or more target site(s) of the subject.

The method can comprise: prior to the administering step, transducing macrophages with a viral vector comprising a circuit disclosed herein, optionally a lentiviral vector to generate an effective amount of the population of heat-inducible macrophages, optionally the method further comprises selecting heat-inducible macrophages that have non undergone the recombination event, optionally heat-inducible macrophages that have non undergone the recombination event are detected via the presence of the detectable protein.

In some embodiments, the expression of the payload from the heat-inducible macrophages is not suppressed by the tumor microenvironment of solid tumor(s). In some embodiments, at least about 20 percent of the population of heat-inducible macrophages are viable 7 days post-administration. In some embodiments, at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the heat-inducible macrophages at the target site express the payload protein after applying thermal energy to the target site. In some embodiments, less than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%, of the heat-inducible macrophages at a site other than the target site express the payload protein. In some embodiments, the ratio of the concentration of payload-expressing heat-inducible macrophages at the subject's target site to the concentration of payload-expressing heat-inducible macrophages in subject's blood, serum, or plasma is at least about 2:1. In some embodiments, the ratio of the concentration of payload protein at the subject's target site to the concentration of payload protein in subject's blood, serum, or plasma is about 2:1 to about 3000:1, about 2:1 to about 2000:1, about 2:1 to about 1000:1, or about 2:1 to about 600:1.

In some embodiments, the target site comprises target cells, optionally the target cells are tumor cells, further optionally the tumor cells are solid tumor cells. In some embodiments, the application of thermal energy to a target site of the subject results in the death of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, of the target cells. In some embodiments, non-target cells comprise cells of the subject other than target cells, and wherein the ratio of target cell death to non-target cell death after application of thermal energy is at least about 2:1. In some embodiments, the ratio of target cell death to non-target cell death is at least about 1.1-fold greater as compared to a method comprising macrophages constitutively expressing the payload protein. In some embodiments, the ratio of the concentration of payload protein at the subject's target site to the concentration of payload protein in a non-target site of the subject is about 2:1 to about 3000:1, about 2:1 to about 2000:1, about 2:1 to about 1000:1, or about 2:1 to about 600:1, optionally said non-target sites comprise the lungs and/or liver.

In some embodiments, the target site comprises a solid tumor. In some embodiments, the target site comprises a site of disease or disorder or is proximate to a site of a disease or disorder, optionally the location of the one or more sites of a disease or disorder is predetermined, is determined during the method, or both, further optionally the target site is an immunosuppressive environment.

In some embodiments, the target site comprises a tissue, optionally the tissue is inflamed tissue and/or infected tissue. In some embodiments, the tissue comprises adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue and/or fat tissue. In some embodiments, the tissue comprises: (i) grade I, grade II, grade III or grade IV cancerous tissue; (ii) metastatic cancerous tissue; (iii) mixed grade cancerous tissue; (iv) a sub-grade cancerous tissue; (v) healthy or normal tissue; and/or (vi) cancerous or abnormal tissue. In some embodiments, the subject is a mammal. In some embodiments, the disease is associated with expression of a tumor antigen, wherein the disease associated with expression of a tumor antigen is selected from the group consisting of a proliferative disease, a precancerous condition, a cancer, and a non-cancer related indication associated with expression of the tumor antigen.

In some embodiments, the disease or disorder is a cancer, optionally a solid tumor. In some embodiments, the cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. In some embodiments, the cancer is a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

The method can comprise: administering an effective amount of pro-death agent and/or a transactivator-binding compound to the subject prior to, during, and/or after administration of the heat-inducible macrophages, optionally the amount of transactivator-binding compound is an amount effective to induce or attenuate a sufficient level of payload expression to treat the subject, further optionally the transactivator-binding compound comprises tetracycline, doxycycline or a derivative thereof. The method can comprise: administering one or more additional agents to the subject, optionally the one or more additional agents increases the efficacy of the heat-inducible macrophages. In some embodiments, the one or more additional agents comprise a protein phosphatase inhibitor, a kinase inhibitor, a cytokine, an inhibitor of an immune inhibitory molecule, or any combination thereof. In some embodiments, the one or more additional agents comprise an immune modulator, an anti-metastatic, a chemotherapeutic, a hormone or a growth factor antagonist, an alkylating agent, a TLR agonist, a cytokine antagonist, a cytokine antagonist, or any combination thereof. In some embodiments, the one or more additional agents comprise an agonistic or antagonistic antibody specific to a checkpoint inhibitor or checkpoint stimulator molecule such as PD1, PD-L1, PD-L2, CD27, CD28, CD40, CD137, OX40, GITR, ICOS, A2AR, B7-H3, B7-H4, BTLA, CTLA4, IDO, KIR, LAG3, PD-1, TIM-3. In some embodiments, administering comprises aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof.

### BRIEF DESCRIPTION OF THE DRAWINGS

FIGS. 1A-1B depict non-limiting exemplary schematics related to the heat-inducible macrophage platforms provided herein. FIG. 1A depicts a non-limiting exemplary schematic showing HSP promoter activation in response to accumulation of unfolded protein activating the expression of Cre recombinase, which catalyzes inversion of DNA flanked by loxP sites. FIG. 1B depicts a non-limiting exemplary schematic illustrating engineered macrophage injection systemically, followed by trafficking of macrophages to various physiological niches. Focused ultrasound can be used to locally elevate temperatures deep within the body in a spatially defined manner. This elevated temperature can activate gene expression in macrophages within a tumour to locally immunomodulate the microenvironment.

FIGS. 2A-2D depict non-limiting exemplary schematics and data related to the construction and evaluation of a thermal state switch in RAW264.7 cells. FIG. 2A depicts a non-limiting exemplary illustration of the genetic circuit constructed to establish a temperature responsive state switch. FIG. 2B depicts data related to the percentage of cells expressing GFP in four separate genetic constructs as a function of thermal induction, measured using flow cytometry. FIG. 2C depicts data related to the percentage of cells expressing GFP as a function of different heating parameters, including temperature and duration, expressed as a ratio of GFP+ cells to viable cells treated with the same heating parameters. Cell viability was normalized using viability stains applied to cells at 37° C. FIG. 2D depicts data related to the percentage of cells expressing either GFP or mScarlet following a thermal stimulus of 43° C. for 15 minutes on day 0. Thresholds for expression of GFP or mScarlet were determined using wild-type RAW264.7 cells. Where not seen, error bars (±SEM) are smaller than the symbol. N=3 biological replicates for each sample.

FIGS. 3A-3E depict non-limiting exemplary schematics and data related to temperature-controlled state switches control the release of CD19-BiTE and IL-12 in RAW264.7 cells. FIG. 3A depicts a non-limiting exemplary illustration of the genetic circuit controlling release of CD-19 BiTE with a temperature responsive state switch. FIG. 3B depicts data showing that following application of the thermal stimulus, RFP expression is switched off while the therapeutic is switched on. After four days, the number of RFP+ cells decreases by nearly 40%. Thresholds for expression of GFP or mScarlet were determined using wild-type RAW264.7 cells. FIG. 3C depicts data related to a competitive ELISA (GenScript) using a C-terminal His-tag on supernatant for detecting secreted CD19 BiTE. N=3 biological replicates for each sample. FIG. 3D depicts a non-limiting exemplary illustration of a genetic circuit controlling release of mouse IL-12 (m-IL-12). This circuit uses a modified design in which an additional fluorescent protein, miRFP, is expressed downstream of the therapeutic. This enables the user to assess inversion of the circuit following heating by flow cytometry. FIG. 3E depicts data related to a IL-12 ELISA (R&D Biosystems) directly detecting mIL12 on supernatant of IL-12 secreting RAW264.7 cells after heating at 42 C for 1 hour.

FIGS. 4A-4C depict non-limiting exemplary schematics and data related to thermal state switch activation using focused ultrasound hyperthermia in vivo. FIG. 4A depicts a non-limiting exemplary illustration of focused ultrasound setup. Temperature probes in the tumor and rectum of the animal are used to monitor tissue heating and body temperature. A closed-loop setup continuously modulates the ultrasound intensity in response to changes in temperature. FIG. 4B depicts data related to the percentage of GFP+ macrophages that have trafficked into Raji tumors following application of focused ultrasound hyperthermia (FUS). Temperatures within the tumor were elevated to 43° C. for 15 minutes, as measured using fiber thermometry. FIG. 4C depicts a representative flow cytometry plot of tumor homogenization 24 hours after HIFU application.

FIGS. 5A-5D depict non-limiting exemplary schematics showing temperature-controlled state switches for the control of macrophage proliferation and prodrug release. FIGS. 5A-5B depict non-limiting exemplary schematics of a genetic circuit controlling expression of iCasp9 (FIG. 5A) and its application in a mouse administered AP20187 (FIG. 5B). FIGS. 5C-5D depict non-limiting exemplary schematics of a genetic circuit controlling expression of HSV-TK (FIG. 5C) and its application in a mouse administered ganciclovir (FIG. 5D).

FIGS. 6A-6D depict non-limiting exemplary schematics and data related to IC-21 heat-inducible macrophages. FIG. 6A depicts a non-limiting exemplary schematic showing a temperature-sensitive state switch genetic circuit design. This circuit was lentivirally transduced into the C57BL/6 mouse peritoneal macrophage cell line, IC-21. FIG. 6B depicts data related to the percent of activated (GFP+) IC-21 cells measured using flow cytometry over 6 days after incubation at 37° C., 42° C., or 43° C. FIG. 6C depicts data related to mean fluorescence intensity of GFP (indicating active state) and RFP (indicating inactive state) of IC-21 cells over 6 days after incubation at 37° C., 42° C., or 43° C. FIG. 6D depicts data related to percent of activated (GFP+) IC-21 cells after incubation at 37° C. or 42° C. for 30 minutes. The cells were treated again 23 days after the initial incubation (indicated by the black arrow).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include populations of heat-inducible macrophages. In some embodiments, one or more of said heat-inducible macrophages comprise: a first inducible promoter operably linked to a first polynucleotide comprising a recombinase gene, wherein the first inducible promoter is capable of inducing transcription of the recombinase gene to generate a recombinase transcript upon thermal stimulation, and wherein the recombinase transcript is capable of being translated to generate a recombinase; and a second promoter and a second polynucleotide comprising a payload gene, wherein, in the absence of a recombination event, the second promoter and the second polynucleotide are not operably linked, wherein the recombinase is capable of catalyzing the recombination event, and wherein the second promoter and the second polynucleotide are operably linked after the recombination event such that the second promoter is capable of inducing transcription of the payload gene to generate a payload transcript.

Disclosed herein include populations of heat-inducible macrophages. In some embodiments, one or more of said heat-inducible macrophages comprise: a first inducible promoter operably linked to a first polynucleotide comprising a transactivator gene, and a second promoter operably linked to a second polynucleotide comprising a payload gene, wherein the first inducible promoter is capable of inducing transcription of the transactivator gene to generate a transactivator transcript in the presence of thermal stimulation, wherein the transactivator transcript is capable of being translated to generate a transactivator; and wherein, in the presence of the transactivator and a transactivator-binding compound, the second promoter is capable of inducing transcription of the payload gene to generate a payload transcript.

Disclosed herein include populations of heat-inducible macrophages. In some embodiments, one or more of said heat-inducible macrophages comprise: a first inducible promoter and a second promoter each operably linked to a first polynucleotide comprising a payload gene and to a second polynucleotide comprising a transactivator gene, wherein the first inducible promoter is capable of inducing transcription of the payload gene and the transactivator gene to generate a polycistronic transcript upon thermal stimulation, wherein, in the presence of the transactivator and a transactivator-binding compound, the second promoter is capable of inducing transcription of the payload gene and the transactivator gene to generate a polycistronic transcript, and wherein the polycistronic transcript is capable of being translated to generate the transactivator and a payload protein and/or payload RNA agent.

Disclosed herein include populations of heat-inducible macrophages. In some embodiments, one or more of said heat-inducible macrophages comprise: a first inducible promoter operably linked to a first polynucleotide comprising a transactivator gene, and a second promoter operably linked to a second polynucleotide comprising a payload gene, wherein the first inducible promoter is capable of inducing transcription of the transactivator gene to generate a transactivator transcript in the presence of thermal stimulation, wherein the second promoter comprises one or more copies of a transactivator recognition sequence the transactivator is capable of binding to induce transcription, and wherein the transactivator is incapable of binding the transactivator recognition sequence in the presence of the transactivator-binding compound, wherein the transactivator transcript is capable of being translated to generate a transactivator; and wherein, in the presence of the transactivator and in the absence of transactivator-binding compound, the second promoter is capable of inducing transcription of the payload gene to generate a payload transcript.

Disclosed herein include populations of heat-inducible macrophages. In some embodiments, one or more of said heat-inducible macrophages comprise: a first inducible promoter and a second promoter each operably linked to a first polynucleotide comprising a payload gene and to a second polynucleotide comprising a transactivator gene, wherein the first inducible promoter is capable of inducing transcription of the payload gene and the transactivator gene to generate a polycistronic transcript upon thermal stimulation, wherein the second promoter comprises one or more copies of a transactivator recognition sequence the transactivator is capable of binding to induce transcription, and wherein the transactivator is incapable of binding the transactivator recognition sequence in the presence of the transactivator-binding compound, wherein, in the presence of the transactivator and in the absence of a transactivator-binding compound, the second promoter is capable of inducing transcription of the payload gene and the transactivator gene to generate a polycistronic transcript, and wherein the polycistronic transcript is capable of being translated to generate the transactivator and a payload protein and/or payload RNA agent.

Disclosed herein include populations of heat-inducible macrophages. In some embodiments, one or more of said heat-inducible macrophages comprise: a first inducible promoter operably linked to a first polynucleotide comprising a payload gene, wherein the first inducible promoter is capable of inducing transcription of the payload gene to generate a payload transcript upon thermal stimulation.

Disclosed herein include methods of treating a disease or disorder in a subject. In some embodiments, the method comprises: administering to the subject an effective amount of a population of heat-inducible macrophages disclosed herein. The method can comprise: applying thermal energy to a target site of the subject sufficient to increase the local temperature of the target site to an activating temperature, thereby inducing the expression of the payload in heat-inducible macrophages at the target site.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). For purposes of the present disclosure, the following terms are defined below.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

The term “vector” as used herein, can refer to a vehicle for carrying or transferring a nucleic acid. Non-limiting examples of vectors include plasmids and viruses (for example, AAV viruses).

The term “construct,” as used herein, refers to a recombinant nucleic acid that has been generated for the purpose of the expression of a specific nucleotide sequence(s), or that is to be used in the construction of other recombinant nucleotide sequences.

As used herein, the term “plasmid” refers to a nucleic acid that can be used to replicate recombinant DNA sequences within a host organism. The sequence can be a double stranded DNA.

The term “element” refers to a separate or distinct part of something, for example, a nucleic acid sequence with a separate function within a longer nucleic acid sequence. The term “regulatory element” and “expression control element” are used interchangeably herein and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Exemplary regulatory elements in prokaryotes include promoters, operator sequences and a ribosome binding sites. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “enhancer” refers to a type of regulatory element that can increase the efficiency of transcription, regardless of the distance or orientation of the enhancer relative to the start site of transcription.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

The term “construct,” as used herein, refers to a recombinant nucleic acid that has been generated for the purpose of the expression of a specific nucleotide sequence(s), or that is to be used in the construction of other recombinant nucleotide sequences.

As used herein, a “subject” refers to an animal that is the object of treatment, observation or experiment. “Animal” includes cold- and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human.

As used herein, the term “treatment” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

As used herein, the term “effective amount” refers to an amount sufficient to effect beneficial or desirable biological and/or clinical results.

“Pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™. Auxiliary, stabilizer, emulsifier, lubricant, binder, pH adjustor controller, isotonic agent and other conventional additives may also be added to the carriers.

The term “antibody fragment” shall be given its ordinary meaning, and shall also refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

The term “autologous” shall be given its ordinary meaning, and shall also refer to any material derived from the same individual to whom it is later to be re-introduced into the individual.

The term “allogeneic” shall be given its ordinary meaning, and shall also refer to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “stimulation,” shall be given its ordinary meaning, and shall also refer to a primary response induced by binding of a stimulatory molecule (e.g., a TCR/CD3 complex or CAR) with its cognate ligand (or tumor antigen in the case of a CAR) thereby mediating a signal transduction event, such as, but not limited to, signal transduction via the TCR/CD3 complex or signal transduction via the appropriate NK receptor or signaling domains of the CAR. Stimulation can mediate altered expression of certain molecules.

### Heat-Inducible Macrophages

The methods and compositions provided herein solve the aforementioned problems in the art and combine spatiotemporal targeting with the ability to penetrate deep into tissues. There are provided, in some embodiments, methods and compositions employing thermally actuated control elements to solve the aforementioned problems in the art, exploiting the ability for temperature to be elevated at arbitrary depth and with high spatial precision using noninvasive methods such as focused ultrasound (FUS). In recent work, it has been shown that temperature responsive bioswitches can be combined with focused ultrasound hyperthermia to control the transcriptional activity of microbes at depth in vivo. In addition, it was recently shown that heat-shock promoters (HSPs) can be assembled with synthetic eukaryotic gene-regulatory elements to create temperature-responsive genetic circuits in immortalized T cells capable of controlling molecular payloads. However, the application of these circuits in vivo in T-cells presents unique challenges. Firstly, T-cells are actively immunosuppressed within solid tumors, which would blunt clinical efficacy, even using a synthetic gene circuit. In addition, the aforementioned work demonstrated that HSPs are strongly activated by T-cell activation, making their usage as ultrasound-controllable therapeutics in T-cells inherently limited in some embodiments. The heat-inducible macrophages provided herein can offer a more effective alternative. Thermal control elements provided herein can be an excellent modality to control the release of therapeutics from systemically administered engineered macrophages harboring temperature-controlled state switches. These engineered macrophages can be actively recruited to tumors. There are provided, in some embodiments, platforms for coupling a brief thermal stimulus to stable gene activation and therapeutic release in macrophages.

Disclosed herein include populations of heat-inducible macrophages. In some embodiments, one or more of said heat-inducible macrophages comprise: a first inducible promoter operably linked to a first polynucleotide comprising a recombinase gene, wherein the first inducible promoter is capable of inducing transcription of the recombinase gene to generate a recombinase transcript upon thermal stimulation, and wherein the recombinase transcript is capable of being translated to generate a recombinase; and a second promoter and a second polynucleotide comprising a payload gene, wherein, in the absence of a recombination event, the second promoter and the second polynucleotide are not operably linked, wherein the recombinase is capable of catalyzing the recombination event, and wherein the second promoter and the second polynucleotide are operably linked after the recombination event such that the second promoter is capable of inducing transcription of the payload gene to generate a payload transcript.

The macrophages can be derived from blood, cord blood, bone marrow, or iPSC. The macrophages can be autologous macrophages and/or allogeneic macrophages. The macrophages can be Kupffer cells, stellate macrophages, M1 macrophages, M2 macrophages, tumor-associated macrophages (TAMs), or any combination thereof.

The recombination event can comprise removal of a sequence flanked by recombinase target sites or an inversion of a sequence flanked by recombinase target sites. The second polynucleotide can be flanked by recombinase target sites. Prior to the recombination event, the sequence of the payload gene can be inverted relative to the promoter. A heat-inducible macrophage can comprise: at least one stop cassette situated between the second promoter and the payload gene, wherein the stop cassette comprises one or more stop sequences, and wherein the one or more stop cassettes are flanked by recombinase target sites. The at least one stop cassette can be configured to prevent transcription of the payload gene and/or translation of the payload transcript. The one or more stop sequences can comprise a polyadenylation signal, a stop codon, a frame-shifting mutation, or any combination thereof.

The second promoter can comprise a ubiquitous promoter. The ubiquitous promoter can be selected from the group comprising a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CBH promoter, or any combination thereof.

The one or more copies of a transactivator recognition sequence can comprise one or more copies of a tet operator (TetO). The second promoter can comprise a tetracycline response element (TRE). The TRE can comprise one or more copies of a tet operator (TetO). The transactivator can comprise reverse tetracycline-controlled transactivator (rtTA). The transactivator can comprise tetracycline-controlled transactivator (tTA). The transactivator-binding compound can comprise tetracycline, doxycycline or a derivative thereof. The first polynucleotide and the second polynucleotide can be operably linked to a tandem gene expression element. The tandem gene expression element can be an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof. The payload protein and the transactivator can be expressed as separate proteins. Tetracycline regulated transcriptional element, or tetracycline transactivator (tTA) is a fusion protein that combines the tetracycline repressor protein (tetR) DNA binding domain with the transcriptional activation domain of VP-16, such that when tTA binds to a minimal promoter containing tetR sequences, transcription of the target gene is activated. Tetracycline binding to tTA prevents activation by causing a conformational change in the tetR portion of tTA which blocks binding of tTA to tetR (Hinrichs, W., et al., (1994) Science 264:418-420); gene activation is achieved by removing tetracycline (Gossen, M. & Bujard, H., (1992) Proc. Natl. Acad. Sci. USA 89:5547-5551). Derivatives or analogues of tetracycline may also be used, including, for example, doxycycline (DOX), minocycline, metacycline, sancycline, chloro-tetracycline, demeclocycline, and tigecycline. Alternatives to rtTA-based transactivators are also contemplated herein, such as, for example, gal4-based transactivators and Cas9-based transactivators. In some embodiments, the transactivator comprises a Cas9 polypeptide (e.g., a dCas9 polypeptide or a dCas12 polypeptide) operably linked to a transcriptional activation domain. In some embodiments, the transcriptional activation domain is selected from the group comprising a VP 16 activation domain, a VP64 activation domain, a p65 activation domain, a MyoDl activation domain, a HSF1 activation domain, a RTA activation domain, a SET7/9 activation domain, a VP64-p65-Rta (VPR) activation domain, a mini VPR activation domain, a yeast GAL4 activation domain, a yeast HAP1 activation domain, a histone acetyltransferase, or any combination thereof. A transactivator recognition sequence can be configured to be recognized by the Cas9 polypeptide. In some embodiments, the second promoter is a TRE3G inducible promoter, a tetracycline-regulated promoter, a steroid-regulated promoter, a metal-regulated promoter, an estrogen receptor-regulated promoter, or a UAS inducible promoter.

The recombinase can be Cre, Dre, Flp, KD, B2, B3, λ, HK022, HP1, γ6, ParA, Tn3, Gin, ΦC31, Bxb1, R4, derivatives thereof, or any combination thereof. The recombinase can be a Flp recombinase and the recombinase target sites can be FRT sites. The recombinase can be a Cre recombinase and the recombinase target sites can be loxP sites. As used herein, the term “lox site” refers to a nucleotide sequence at which the product of the ere gene of bacteriophage PI, Cre recombinase, can catalyze a site-specific recombination. A variety of lox sites are known to the art including but not limited to the naturally occurring loxP (the sequence found in the PI genome), loxB, loxL and loxR (these are found in the E. coli chromosome) as well as a number of mutant or variant lox sites such as loxP511, lox2272, loxA86, loxA117, loxC2, loxP2, loxP3 and loxP23. The term “frt site” as used herein refers to a nucleotide sequence at which the product of the FLP gene of the yeast 2 pm plasmid, FLP recombinase, can catalyze a site-specific recombination.

The term “recombinase,” as used herein, refers to a site-specific enzyme that mediates the recombination of DNA between recombinase recognition sequences (e.g., recombinase target sites), which results in the excision, integration, inversion, or exchange (e.g., translocation) of DNA fragments between the recombinase recognition sequences. Recombinases can be classified into two distinct families: serine recombinases (e.g., resolvases and invertases) and tyrosine recombinases (e.g., integrases). Examples of serine recombinases include, without limitation, Hin, Gin, Tn3, β-six, CinH, ParA, yδ, Bxb1, ϕC31, TP901, TG1, φBT1, R4, φRV1, φFC1, MR11, A118, U153, and gp29. Examples of tyrosine recombinases include, without limitation, Cre, FLP, R, Lambda, HK101, HK022, and pSAM2. The term “recombine” or “recombination,” in the context of a nucleic acid modification (e.g., a genomic modification), is used to refer to the process by which two or more nucleic acid molecules, or two or more regions of a single nucleic acid molecule, are modified by the action of a recombinase protein. Recombination can result in, inter alia, the insertion, inversion, excision, or translocation of a nucleic acid sequence, e.g., in or between one or more nucleic acid molecules. As used herein, the term “recombination site sequences” or “recombinase target sites” refers to short polynucleic acid sequences, typically palindromic, that are specifically recognized and acted upon by a DNA recombinase. DNA recombinase/recombination site sequence pairs include, but are not limited to, Cre/loxP, Dre/rox, VCre/VloxP, SCre/SloxP, Vika/vox, λ-int/attP, Flp/FRT, R/RRT, Kw/KwRT, Kd/KdRT, B2/B2RT, and B3/B3RT.

A heat-inducible macrophage can comprise: a third polynucleotide comprising a default gene. In some embodiments, in the absence of a recombination event, the second promoter and the third polynucleotide are operably linked such that the second promoter is capable of inducing transcription of the default gene to generate a default transcript. The default transcript can be capable of being translated to generate a default protein. In some embodiments, the second promoter and the third polynucleotide are not operably linked after the recombination event such that the second promoter is no longer capable of inducing transcription of the default gene to generate a default transcript. In some embodiments, default gene encodes a detectable protein. The detectable protein can comprise green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mScarlet, mApple, mCitrine, mCherry, mruby3, rsCherry, rsCherryRev, derivatives thereof, or any combination thereof.

The default gene can be a pro-death gene encoding a pro-death protein. The pro-death protein can be capable of halting cell growth and/or inducing cell death. The third polynucleotide can further comprise a detectable gene encoding a detectable protein. The detectable protein can comprise green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mScarlet, mApple, mCitrine, mCherry, mruby3, rsCherry, rsCherryRev, derivatives thereof, or any combination thereof. The detectable gene and the pro-death gene can be operably linked to a tandem gene expression element. The tandem gene expression element can be an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof.

The payload protein can be a pro-death protein. The pro-death protein can be capable of halting cell growth and/or inducing cell death. The pro-death protein can comprise a secretion tag. The secretion tag can be selected from the group comprising AbnA, AmyE, AprE, BglC, BglS, Bpr, Csn, Epr, Ggt, GlpQ, HtrA, LipA, LytD, MntA, Mpr, NprE, OppA, PbpA, PbpX, Pel, PelB, PenP, PhoA, PhoB, PhoD, PstS, TasA, Vpr, WapA, WprA, XynA, XynD, YbdN, Ybxl, YcdH, YclQ, YdhF, YdhT, YfkN, YflE, YfmC, Yfnl, YhcR, YlqB, YncM, YnfF, YoaW, YocH, YolA, YqiX, Yqxl, YrpD, YrpE, YuaB, Yurl, YvcE, YvgO, YvpA, YwaD, YweA, YwoF, YwtD, YwtF, YxaLk, YxiA, and YxkC. In some embodiments, heat-inducible macrophages that have undergone the recombination event are capable of secreting said pro-death protein and killing bystander tumor cells.

The pro-death protein can comprise cytosine deaminase, iCasp9, thymidine kinase, Bax, Bid, Bad, Bak, BCL2L11, p53, PUMA, Diablo/SMAC, S-TRAIL, Cas9, Cas9n, hSpCas9, hSpCas9n, HSVtk, cholera toxin, diphtheria toxin, alpha toxin, anthrax toxin, exotoxin, pertussis toxin, Shiga toxin, shiga-like toxin Fas, TNF, caspase 2, caspase 3, caspase 6, caspase 7, caspase 8, caspase 9, caspase 10, caspase 11, caspase 12, purine nucleoside phosphorylase, or any combination thereof. The pro-death protein can be capable of halting cell growth and/or inducing cell death in the presence of a pro-death agent. In some embodiments, pro-death protein can comprise iCasp9 and the pro-death agent can comprise AP20187. The pro-death protein can comprise Caspase-9 and the pro-death agent can comprise AP1903. The pro-death protein can comprise HSV thymidine kinase (TK) and the pro-death agent can comprise Ganciclovir (GCV), Ganciclovir elaidic acid ester, Penciclovir (PCV), Acyclovir (ACV), Valacyclovir (VCV), (E)-5-(2-bromovinyl)-2′-deoxyuridine (BVDU), Zidovuline (AZT), and/or 2′-exo-methanocarbathymidine (MCT). The pro-death protein can comprise Cytosine Deaminase (CD) and the pro-death agent can comprise 5-fluorocytosine (5-FC). The pro-death protein can comprise Purine nucleoside phosphorylase (PNP) and the pro-death agent can comprise 6-methylpurine deoxyriboside (MEP) and/or fludarabine (FAMP). The pro-death protein can comprise a Cytochrome p450 enzyme (CYP) and the pro-death agent can comprise Cyclophosphamide (CPA), Ifosfamide (IFO), and/or 4-ipomeanol (4-IM). The pro-death protein can comprise a Carboxypeptidase (CP) and the pro-death agent can comprise 4-[(2-chloroethyl)(2-mesyloxyethyl)amino]benzoyl-L-glutamic acid (CMDA), Hydroxy-and amino-aniline mustards, Anthracycline glutamates, and/or Methotrexate α-peptides (MTX-Phe). The pro-death protein can comprise Carboxylesterase (CE) and the pro-death agent can comprise Irinotecan (IRT), and/or Anthracycline acetals. The pro-death protein can comprise Nitroreductase (NTR) and the pro-death agent can comprise dinitroaziridinylbenzamide CB1954, dinitrobenzamide mustard SN23862, 4-Nitrobenzyl carbamates, and/or Quinones. The pro-death protein can comprise Horse radish peroxidase (HRP) and the pro-death agent can comprise Indole-3-acetic acid (IAA) and/or 5-Fluoroindole-3-acetic acid (FIAA). The pro-death protein can comprise Guanine Ribosyltransferase (XGRTP) and the pro-death agent can comprise 6-Thioxanthine (6-TX). The pro-death protein can comprise a glycosidase enzyme and the pro-death agent can comprise HM1826 and/or Anthracycline acetals. The pro-death protein can comprise Methionine-α,γ-lyase (MET) and the pro-death agent can comprise Selenomethionine (SeMET). The pro-death protein can comprise thymidine phosphorylase (TP) and the pro-death agent can comprise 5′-Deoxy-5-fluorouridine (5′-DFU).

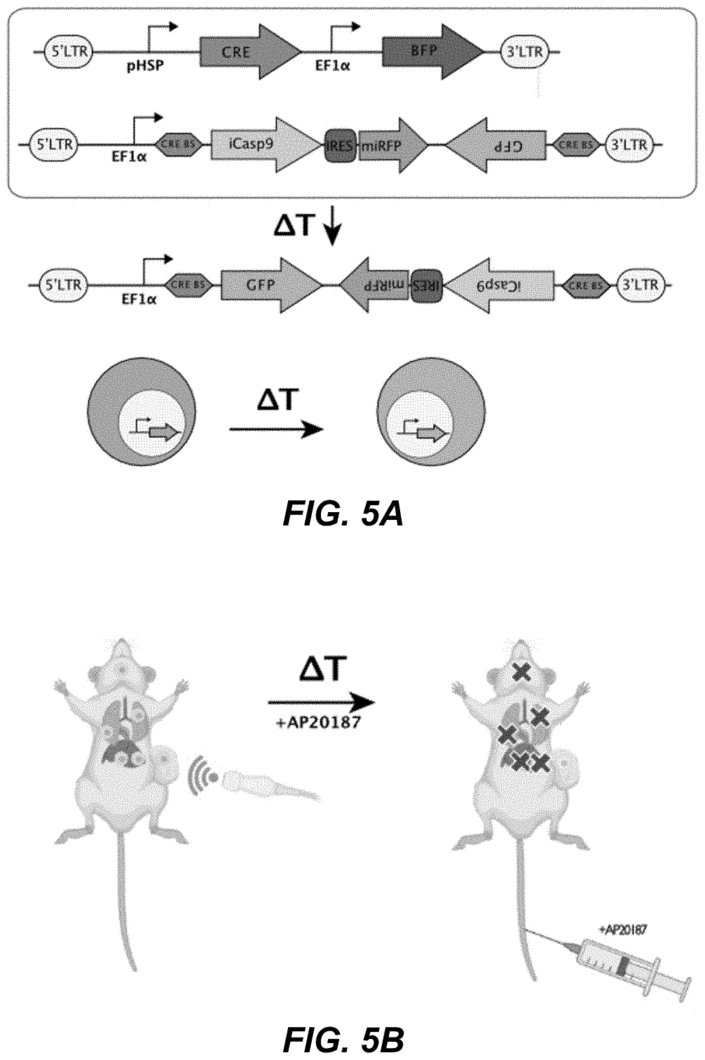

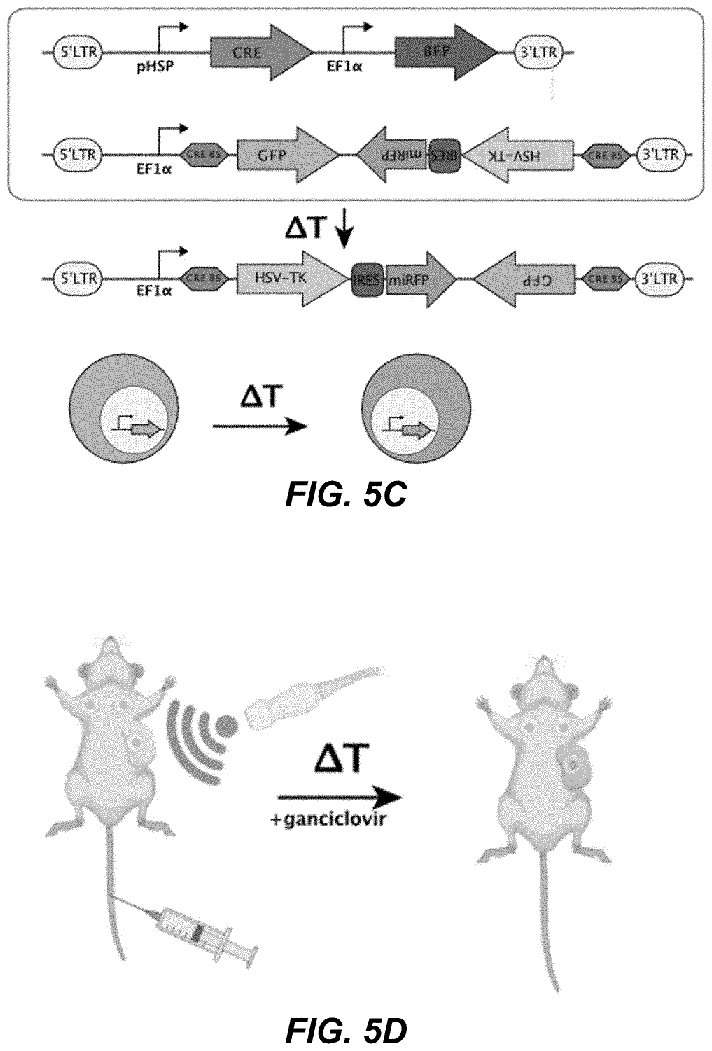

FIGS. 5A-5D depict non-limiting exemplary schematics showing temperature-controlled state switches for the control of macrophage proliferation and prodrug release. FIG. 5A depicts non-limiting exemplary schematic of a genetic circuit controlling expression of iCasp9. iCasp9 is a drug inducible caspase switch that executes an apoptotic program. In the absence of the inducer, AP20187, iCasp9 remains off and cells are able to survive and proliferate (FIG. 5B). After cells are injected systemically, focused ultrasound is used to switch the iCasp9 gene off. Following dilution of the iCasp9 through cell division, AP20187 is injected into the mouse. Only cells that have received focused ultrasound hyperthermia are able to survive the addition of the drug, as all cells which have not undergone inversion will undergo apoptosis. This switch may be used to prevent proliferation of an engineered cell line outside of the tumor. FIG. 5C depicts non-limiting exemplary schematic of a genetic circuit controlling expression of HSV-TK. HSV-TK is a thymidine kinase that converts ganciclovir, a prodrug into a toxic metabolite (GCVTP). Once focused ultrasound hyperthermia is applied, ganciclovir is administered systemically (FIG. 5D). Only cells that have received focused ultrasound hyperthermia are able to convert ganciclovir into the toxic metabolite as a result of recombinase expression. This metabolite is able to kill bystander tumor cells once it is transported out of the engineered macrophage. In some embodiments, the methods and compositions provided herein employ one or more elements of the circuits and methods depicted in FIGS. 5A-5D.

Disclosed herein include populations of heat-inducible macrophages. In some embodiments, one or more of said heat-inducible macrophages comprise: a first inducible promoter operably linked to a first polynucleotide comprising a transactivator gene, and a second promoter operably linked to a second polynucleotide comprising a payload gene, wherein the first inducible promoter is capable of inducing transcription of the transactivator gene to generate a transactivator transcript in the presence of thermal stimulation, wherein the transactivator transcript is capable of being translated to generate a transactivator; and wherein, in the presence of the transactivator and a transactivator-binding compound, the second promoter is capable of inducing transcription of the payload gene to generate a payload transcript.

Disclosed herein include populations of heat-inducible macrophages. In some embodiments, one or more of said heat-inducible macrophages comprise: a first inducible promoter and a second promoter each operably linked to a first polynucleotide comprising a payload gene and to a second polynucleotide comprising a transactivator gene, wherein the first inducible promoter is capable of inducing transcription of the payload gene and the transactivator gene to generate a polycistronic transcript upon thermal stimulation, wherein, in the presence of the transactivator and a transactivator-binding compound, the second promoter is capable of inducing transcription of the payload gene and the transactivator gene to generate a polycistronic transcript, and wherein the polycistronic transcript is capable of being translated to generate the transactivator and a payload protein and/or payload RNA agent.

In some embodiments, the second promoter comprises one or more copies of a transactivator recognition sequence the transactivator is capable of binding to induce transcription, and wherein the transactivator is incapable of binding the transactivator recognition sequence in the absence of the transactivator-binding compound. The one or more copies of a transactivator recognition sequence can comprise one or more copies of a tet operator (TetO).

Disclosed herein include populations of heat-inducible macrophages. In some embodiments, one or more of said heat-inducible macrophages comprise: a first inducible promoter operably linked to a first polynucleotide comprising a transactivator gene, and a second promoter operably linked to a second polynucleotide comprising a payload gene, wherein the first inducible promoter is capable of inducing transcription of the transactivator gene to generate a transactivator transcript in the presence of thermal stimulation, wherein the second promoter comprises one or more copies of a transactivator recognition sequence the transactivator is capable of binding to induce transcription, and wherein the transactivator is incapable of binding the transactivator recognition sequence in the presence of the transactivator-binding compound, wherein the transactivator transcript is capable of being translated to generate a transactivator; and wherein, in the presence of the transactivator and in the absence of transactivator-binding compound, the second promoter is capable of inducing transcription of the payload gene to generate a payload transcript.

Disclosed herein include populations of heat-inducible macrophages. In some embodiments, one or more of said heat-inducible macrophages comprise: a first inducible promoter and a second promoter each operably linked to a first polynucleotide comprising a payload gene and to a second polynucleotide comprising a transactivator gene, wherein the first inducible promoter is capable of inducing transcription of the payload gene and the transactivator gene to generate a polycistronic transcript upon thermal stimulation, wherein the second promoter comprises one or more copies of a transactivator recognition sequence the transactivator is capable of binding to induce transcription, and wherein the transactivator is incapable of binding the transactivator recognition sequence in the presence of the transactivator-binding compound, wherein, in the presence of the transactivator and in the absence of a transactivator-binding compound, the second promoter is capable of inducing transcription of the payload gene and the transactivator gene to generate a polycistronic transcript, and wherein the polycistronic transcript is capable of being translated to generate the transactivator and a payload protein and/or payload RNA agent.

The one or more copies of a transactivator recognition sequence can comprise one or more copies of a tet operator (TetO). The second promoter can comprise a tetracycline response element (TRE), and the TRE can comprise one or more copies of a tet operator (TetO). The transactivator can comprise reverse tetracycline-controlled transactivator (rtTA). The transactivator can comprise tetracycline-controlled transactivator (tTA). The transactivator-binding compound can comprise tetracycline, doxycycline or a derivative thereof. The first polynucleotide and the second polynucleotide can be operably linked to a tandem gene expression element. The tandem gene expression element can be an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof. The payload protein and the transactivator can be expressed as separate proteins.

Disclosed herein include populations of heat-inducible macrophages. In some embodiments, one or more of said heat-inducible macrophages comprise: a first inducible promoter operably linked to a first polynucleotide comprising a payload gene, wherein the first inducible promoter is capable of inducing transcription of the payload gene to generate a payload transcript upon thermal stimulation.

The payload transcript can be capable of being translated to generate a payload protein. Thermal stimulation can comprise heating to an activating temperature. The activating temperature can be about 37.5° C., about 38.0° C., about 38.5° C., about 39.0° C., about 39.5° C., about 40.0° C., about 40.5° C., about 41.0° C., about 41.5° C., about 42.0° C., about 42.5° C., about 43.0° C., about 43.5° C., about 44.0° C., about 44.5° C., about 45.0° C., about 45.5° C., or about 46.0° C.

In some embodiments, in the absence of thermal stimulation, the payload protein reaches unstimulated steady state payload protein levels in the heat-inducible macrophage. The unstimulated steady state payload protein levels can be insufficient to exert a phenotypic effect and/or therapeutic effect on said heat-inducible macrophage and/or target cells (e.g., target cells at a target site). In some embodiments, upon thermal stimulation, transcription of the payload gene, transactivator gene, and/or recombinase gene from the first inducible promoter is increased by at least 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, or a number or a range between any of these values). In some embodiments, the steady-state levels of the payload transcript, the steady-state levels of transactivator transcript, the steady-state levels of recombinase transcript, and/or the steady-state levels of the polycistronic transcript are at least 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, or a number or a range between any of these values) higher upon thermal stimulation. In some embodiments, upon thermal stimulation, the payload protein reaches stimulated steady state payload protein levels in the heat-inducible macrophage. In some embodiments, the payload protein does not return to unstimulated steady state payload protein levels; and/or the payload protein does not return to unstimulated steady state payload protein levels for at least about 4 weeks, about 3 weeks, about 2 weeks, about 1 week, about 6 days, about 5 days, about 96 hours, about 48 hours, about 44 hours, about 40 hours, about 35 hours, about 30 hours, about 25 hours, 20 hours, 15 hours, 10 hours, about 8 hours, about 8 hours, 8 hours, about 7 hours, about 6 hours, about 5 hours, about 4 hours, about 3 hours, about 2 hours, about 1 hour, about 30 minutes, about 15 minutes, about 10 minutes, or about 5 minutes. Stimulated steady state payload protein levels can be at least 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, or a number or a range between any of these values) higher than unstimulated steady state payload protein levels. In some embodiments, increasing transactivator-binding compound concentration increases stimulated steady state payload protein levels. The systems, methods, compositions, and kits provided herein can, in some embodiments, be employed in concert with the systems, methods, compositions, and kits described in U.S. patent application Ser. No. 17/866,240 & 17/230,998, the contents of which are incorporated herein by reference in their entireties.

Inducible Promoters

There are provided, in some embodiments, promoters capable of inducing transcription upon thermal stimulation (e.g., inducible promoters). The first inducible promoter can comprise or can be derived from a mammalian heat shock promoter (HSP) or a C. elegans HSP. The mammalian HSP can be a human HSP or mouse HSP. The first inducible promoter can comprise a nucleotide sequence that is at least 80%, 85%, 90%, 95%, 98%, 99%, or 100% identical to any one of SEQ ID NOS: 1-14. The first inducible promoter can comprise one or more AP-1 sites. In some embodiments, the first inducible promoter does not comprise an AP-1 site. The first inducible promoter can comprise a bidirectional promoter and/or a minimal bidirectional promoter. The first inducible promoter can comprise one or more heat shock element (HSE) binding sites (e.g., four HSE binding sites). In some embodiments, the first inducible promoter does not comprise a human transcription factor binding site other than one or more HSE binding sites. In some embodiments, the first inducible promoter comprises one or more of a TATA box, GC-Box, CAAT signal, and AP-1 site. Nucleic acids provided herein can comprise a portion of a promoter, an enhancer, positive or negative cis-acting sequences, inducible or repressible control element, 5′ UTR sequences that are upstream of a gene, or any combination thereof. A disclosed promoter (e.g., first inducible promoter, second promoter) can comprise 0, 1, 2, 3, 4, 5, 6, 7, 8, 9, 10, 11, 12, 13, 14, 15, 16, 17, 18, 19, or 20 HSE binding sites. The inducible promoter can comprise a promoter sequence shown in Table 1.

A disclosed promoter (e.g., first inducible promoter, second promoter) can be derived from the heat shock promoter (HSP) of one or more species selected from the group comprising: Arabidopsis thaliana; Aspergillus nidulans; Bombyx mori; Candida albicans; Caenorhabditis elegans; Chlamydomonas rheinhardtii; Cricetulus griseus; Cyanophora paradoxa; Cylindrotheca fusiformis; Danio rerio; Dictyostelium discoideum; Drosophila melanogaster; Drosophila yakuba; Gallus gallus; Homo Sapiens; Leishmania chagasi; Leishmania major; Loligo pealii; Lymantria dispar; Monodelphis domestica; Morone saxatilis; Mus musculus; Nectria haematococca; Neurospora crassa; Nicotiana tabacum; Oryza sativa; Paracentrotus lividus; Plasmodium falciparum; Rattus norvegicus; Saccharomyces cerevisiae; Schizosaccharomyces pombe; Solanum tuberosum; Strongylocentrotus purpuratus; Syncephalastrum racemosum; Tetrahymena thermophila; Trypanosoma brucei; Ustilago maydis; Volvox carteri; and Xenopus laevis.

The length of the promoters provided herein (e.g., first inducible promoter, second promoter) can vary. In some embodiments, a disclosed promoter (e.g., first promoter, second promoter) is, or is about, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1050, 1100, 1150, 1200, 1250, 1300, 1350, 1400, 1450, 1500, 1550, 1600, 1650, 1700, 1750, 1800, 1850, 1900, 1950, 1100, 1150, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, or 4000, or a number or a range between any two of these values, nucleotides in length. In some embodiments, a disclosed promoter (e.g., first promoter, second promoter) is at least, or is at most, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1050, 1100, 1150, 1200, 1250, 1300, 1350, 1400, 1450, 1500, 1550, 1600, 1650, 1700, 1750, 1800, 1850, 1900, 1950, 1100, 1150, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, or 4000, nucleotides in length.

In some embodiments, the sequence identity between a disclosed promoter (e.g., first inducible promoter, second promoter) and the sequence of any one of SEQ ID NOS: 1-14 can be, or be about, 0.000000001%, 0.00000001%, 0.0000001%, 0.000001%, 0.00001%, 0.0001%, 0.001%, 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values. In some embodiments, the sequence identity between a disclosed promoter (e.g., first promoter, second promoter) and the sequence of any one of SEQ ID NOS: 1-14 can be at least, or at most, 0.000000001%, 0.00000001%, 0.0000001%, 0.000001%, 0.00001%, 0.0001%, 0.001%, 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

A disclosed promoter (e.g., first inducible promoter, second promoter) can comprise at least about 20 consecutive nucleotides (e.g., about 20 nt, 25 nt, 30 nt, 35 nt, 40 nt, 45 nt, 50 nt, 60 nt, 70 nt, 80 nt, 90 nt, 100 nt, 110 nt, 120 nt, 128 nt, 130 nt, 140 nt, 150 nt, 160 nt, 170 nt, 180 nt, 190 nt, 200 nt, 210 nt, 220 nt, 230 nt, 240 nt, 250 nt, 260 nt, 270 nt, 280 nt, 290 nt, 300 nt, 310 nt, 320 nt, 330 nt, 340 nt, 350 nt, 360 nt, 370 nt, 380 nt, 390 nt, 400 nt, 410 nt, 420 nt, 430 nt, 440 nt, 450 nt, 460 nt, 470 nt, 480 nt, 490 nt, 500 nt, 510 nt, 520 nt, 530 nt, 540 nt, 550 nt, 560 nt, 570 nt, 580 nt, 590 nt, 600 nt, 610 nt, 620 nt, 630 nt, 640 nt, 650 nt, 660 nt, 670 nt, 680 nt, 690 nt, 700 nt, 710 nt, 720 nt, 730 nt, 740 nt, 750 nt, 760 nt, 770 nt, 780 nt, 790 nt, 800 nt, 810 nt, 820 nt, 830 nt, 840 nt, 850 nt, 860 nt, 870 nt, 880 nt, 890 nt, 900 nt, 910 nt, 920 nt, 930 nt, 940 nt, 950 nt, 960 nt, 970 nt, 980 nt, 990 nt, 1000 nt, or a number or a range between any two of these values) of a sequences described by SEQ ID NOS: 1-14.

TABLE 1
pHSP Sequences

| NAME | SEQUENCE | SEQ ID NO: |
|---|---|---|
| HSPB | AATTAGCTTGAggatcctccacagccccggggagaccttgcctctaaagt | SEQ ID NO: 1 |
| | tgctgcttttgcagctctgccacaaccgcgcgtcctcagagccagccggg | |
| | aggagctagaaccttccccgcgtttctttcagcagccctgagtcagaggc | |
| | gggctggccttgcaagtagccgcccagccttcttcggtctcacggaccga | |
| | tccgcccgaaccttctcccggggtcagcgccgcgctgcgccgcccggctg | |
| | actcagcccgggcgggcgggcgggaggctctcgactgggcgggaaggtgc | |
| | gggaaggttcgcggcggcggggtcggggaggtgcaaaaggatgaaaagcc | |
| | cgtggacggagctgagcagatccggccgggctggcggcagagaaaccgca | |
| | gggagagcctcactgctgagcgcccctcgacgcgggcggcagcagcctcc | |
| | gtggcctccagcatccgacaagaagcttcagcc | |
| HSPB'1 | AATTAGCTTGAGCCTCTAAAGTTGCTGCTTTTGCAGCCTCTGCCACAACC | SEQ ID NO: 2 |
| | GCGCGTCCTCAGAGCCAGCCCGGAGGAGCTAGAACCTTCCCCGCATTTCT | |
| | TTCAGCAGCCTGAGTCAGAGGCGGGCTGGCCTGGCGTAGCCGCCCAGCCT | |
| | CGCGGCTCATGCCCCGATCTGCCCGAACCTTCTCCCGGGGTCAGCGCCGC | |
| | GCCGCGCCACCCGGCTGAGTCAGCCCGGGCGGGCGAGAGGCTCTCAACTG | |
| | GGCGGGAAGGTGCGGGAAGGTGCGGAAAGGTTCGCGAAAGTTCGCGGCGG | |
| | CGGGGGTCGGGTGAGGCGCAAAAGGATAAAAAGCCggtggaagcggaGCT | |
| | GAGCAGATCCGAGCCGGGCTGGCTGCAGAGAAACCGCAGGGAGAGCCTCA | |
| | CTGCTGAGCGCCCCTCGACGGCGGAGCGGCAGCAGCCTCCGTGGCCTCCA | |
| | GCATCCGACAAGAAGCTTGAATTCGAGCTCGCCGGGGATCCTCTAGTCAG | |
| | CTGACGCGTGCTAGCGCGGCCGCACCACTAGTGCCACC | |
| HSPB'2 | AATTAGCTTGAGCCTCTAAAGTTGCTGCTTTTGCAGCCTCTGCCACAACC | SEQ ID NO: 3 |
| | GCGCGTCCTCAGAGCCAGCCCGGAGGAGCTAGAACCTTCCCCGCATTTCT | |
| | TTCAGCAGCCTGAGTCAGAGGCGGGCTGGCCTGGCGTAGCCGCCCAGCCT | |
| | CGCGGCTCATGCCCCGATCTGCCCGAACCTTCTCCCGGGGTCAGCGCCGC | |
| | GCCGCGCCACCCGGCTGAGTCAGCCCGGGCGGGCGAGAGGCTCTCAACTG | |
| | GGCGGGAAGGTGCGGGAAGGTGCGGAAAGGTTCGCGAAAGTTCGCGGCGG | |
| | CGGGGGTCGGGTGAGGCGCAAAAGGATAAAAAGCCggtggaagcggaGCT | |
| | GAGCAGATCCGAGCCGGGCTGGCTGCAGAGAAACCGCAGGGAGAGCCTCA | |
| | CTGCTGAGCGCCCCTCGACGGCGGAGCGGCAGCAGCCTCCGTGGCCTCCA | |
| | GCATCCGACAAGAAGCTTGAATTCGAGCTCGCCGGGGATCCTCTAGTCAG | |
| | CTGACGCGTGCTAGCGCGGCGCCACC | |
| HSPB‘3 | AATTAGCTTGAGCCTCTAAAGTTGCTGCTTTTGCAGCCTCTGCCACAACC | SEQ ID NO: 4 |
| | GCGCGTCCTCAGAGCCAGCCCGGAGGAGCTAGAACCTTCCCCGCATTTCT | |
| | TTCAGCAGCCTGAGTCAGAGGCGGGCTGGCCTGGCGTAGCCGCCCAGCCT | |
| | CGCGGCTCATGCCCCGATCTGCCCGAACCTTCTCCCGGGGTCAGCGCCGC | |
| | GCCGCGCCACCCGGCTGAGTCAGCCCGGGCGGGCGAGAGGCTCTCAACTG | |
| | GGCGGGAAGGTGCGGGAAGGTGCGGAAAGGTTCGCGAAAGTTCGCGGCGG | |
| | CGGGGGTCGGGTGAGGCGCAAAAGGATAAAAAGCCggtggaagcggaGCT | |
| | GAGCAGATCCGAGCCGGGCTGGCTGCAGAGAAACCGCAGGGAGAGCCTCA | |
| | CTGCTGAGCGCCCCTCGACGGCGGAGCGGCAGCAGCCTCCGTGGCCTCCA | |
| | GCATCCGACAAGAAGCTTcagCC | |
| SynHSPB'l | AATTAGCTTGACCCCGATCTGCCCGAACCTTCTCCCGGGGTCAGCGCCGC | SEQ ID NO: 5 |
| | GCCGCGCCACCCGGCTGAGTCAGCCCGGGCGGGCGAGAGGCTCTCAACTG | |
| | GGCGGGAAGGTGCGGGAAGGTGCGGAAAGGTTCGCGAAAGTTCGCGGCGG | |
| | CGGGGGTCGGGTGAGGCGCAAAAGGATAAAAAGCCggtggaagcggaGCT | |
| | GAGCAGATCCGAGCCGGGCTGGCTGCAGAGAAACCGCAGGGAGAGCCTCA | |
| | CTGCTGAGCGCCCCTCGACGGCGGAGCGGCAGCAGCCTCCGTGGCCTCCA | |
| | GCATCCGACAAGAAGCTTcagCC | |
| SynHSPB'2 | AATTAGCTTGACCCCGATCTGCCCGAACCTTCTCCCGGGGTCAGCGCCGC | SEQ ID NO: 6 |
| | GCCGCGCCACCCGGCTGCAGCAGCCCGGGCGGGCGAGAGGCTCTCAACTG | |
| | GGCGGGAAGGTGCGGGAAGGTGCGGAAAGGTTCGCGAAAGTTCGCGGCGG | |
| | CGGGGGTCGGGTGAGGCGCAAAAGGATAAAAAGCCggtggaagcggaGCT | |
| | GAGCAGATCCGAGCCGGGCTGGCTGCAGAGAAACCGCAGGGAGAGCCTCA | |
| | CTGCTGAGCGCCCCTCGACGGCGGAGCGGCAGCAGCCTCCGTGGCCTCCA | |
| | GCATCCGACAAGAAGCTTcagCC | |
| SynHSPB'3 | AATTAGCTTGACCCCGATCTGCCCGAACCTTCTCCCGGGGTCAGCGCCGC | SEQ ID NO: 7 |
| | GCCGCGCCACCCGGCTGCAGCAGCCCGGGCGGGCGAGAGGCTCTCAACTG | |
| | GGCGGGAAGGTGCGGGAAGGTGCGGAAAGGTTCGCGAAAGTTCGCGGCCG | |
| | GACTAGAGTGGCGAGATCCCCCGATCTGCCCGAACCTTCTCCCGGGGTCA | |
| | GCGCCGCGCCGCGCCACCCGGCTGCAGCAGCCCGGGCGGGCGAGAGGCTC | |
| | TCAACTGGGCGGGAAGGTGCGGGAAGGTGCGGAAAGGTTCGCGAAAGTTC | |
| | GCGGCAATTAGCTTGACCCCGATCTGCCCGAACCTTCTCCCGGGGTCAGC | |
| | GCCGCGCCGCGCCACCCGGCTGCAGCAGCCCGGGCGGGCGAGAGGCTCTC | |
| | AACTGGGCGGGAAGGTGCGGGAAGGTGCGGAAAGGTTCGCGAAAGTTCGC | |
| | GGCGGCGGGGGTCGGGTGAGGCGCAAAAGGATAAAAAGCCggtggaagcg | |
| | gaGCTGAGCAGATCCGAGCCGGGCTGGCTGCAGAGAAACCGCAGGGAGAG | |
| | CCTCACTGCTGAGCGCCCCTCGACGGCGGAGCGGCAGCAGCCTCCGTGGC | |
| | CTCCAGCATCCGACAAGAAGCTTcagCC | |
| HSPA/A | AATTAGCTTGAGCCGCCCACTCCCCCTTCCTCTCAGGGTCCCTGTCCCCT | SEQ ID NO: 8 |
| | CCAGTGAATCCCAGAAGACTCTGGAGAGTTCTGAGCAGGGGGCGGCACTC | |
| | TGGCCTCTGATTGGTCCAAGGAAGGCTGGGGGGCAGGACGGGAGGCGAAA | |
| | ACCCTGGAATATTCCCGACCTGGCAGCCTCATCGAGCTCGGTGATTGGCT | |
| | CAGAAGGGAAAAGGCGGGTCTCCGTGACGACTTATAAAAGCCCAGGGGCA | |
| | AGCGGTCCGGATAACGGCTAGCCTGAGGAGCTGCTGCGACAGTCCACTAC | |
| | CTTTTTCGAGAGTGACTCCCGTTGTCCCAAGGCTTCCCAGAGCGAACCTG | |
| | TGCGGCTGCAGGCACCGGCGCGTCGAGTTTCCGGCGTCCGGAAGGACCGA | |
| | GCTCTTCTCGCGGATCCAGTGTTCCGTTTCCAGCCCCCAATCTCAGAGCG | |
| | GAGCCGACAGAGAGCAGGGAACCgCC | |
| HSPA/B | AATTAGCTTGActccttcccattaagacggaaaaaacatccgggagagcc | SEQ ID NO: 9 |
| | ggtccgtttctcaggcagactaggccattaggtgcctcggagaaaggacc | |
| | caaggctgctccgtccttcacagacacagtccaatcagagtttcccaggc | |
| | acatcgatgcaccgcctccttcgagaaacaaggtaactttcgggttctgg | |
| | ttgtctccaaagtcatccgaccaatctcgcaccgcccagagcgggccctt | |
| | cctgtcaattacctactgaagggcaggcggccagcatcgccatggagacc | |
| | aacacccttcccaccaccactccccctttctctcagggcccctgtcccct | |
| | ccagtgaatcccagaagactctggagagttctgagcagagggcggcaccc | |
| | tgccctctgattggtccaaggaaggctggggggcaggacgggaggcgaaa | |
| | cccctggaatattcccgacctggcagcctcatcgagcttggtgattggct | |
| | cagaaggggaaaggcgggtctccacgacgacttataaaagccgaggggcg | |
| | egcggtccggaaaacggccageetgaggagctgctgcgagggtcegette | |
| | gtctttcgagagtgactcccgcggtcccaaggctttccagagcgaacctg | |
| | tgcggctgcaggcaccggcgtgttgagtttccggcgttccgaaggactga | |
| | gctcttgtcgcggatcccgtccgccgtttccagcccccagtctcagagcg | |
| | gagcccacagagcagggcaccggc | |
| HSPm1 | AATTAGCTTGAAAATCAGTCAAACCTAAGAAAATTCTCaaccgcatcaaa | SEQ ID NO: 10 |
| | ccgaggaccaactgggacacagagcttctgccccactccaatcagagcct | |
| | tcccagctcacctgggatctctacgccttcgatccagtttggaaaatttc | |
| | aagtegetgageccctacgagaggagctccaggaacataccaaactgagg | |
| | cagccggggtcccccccaccccccaccccgcccctcccggcaactttgag | |
| | cctgtgctgggacagagcctctagttcctaaattagtccatgaggtcaga | |
| | ggcagcactgccattgtaaccgcgattggagaggatcacgtcaccggaca | |
| | cgccccaggcatctccctgggtctcctaaacttggccggggagaagtttt | |
| | agcccttaaggttttagcctttaacccccatattcagaactgtgcgagtt | |
| | ggcgaaaccccacaaatcacaacaaactgtacacaacaccgaggctagag | |
| | gtgatctttcttgtccattccacacaggccttagtaattgcgtcgccata | |
| | gcaacagtgtcactagtagcaccagcacgttccccacaccctccccctca | |
| | ggaatccgtactctccagtgaaccccagaaacctctggagagttctggac | |
| | aagggcggaacccacaactccgattactcaagggaggcggggaagctcca | |
| | ccagacgcgaaactgctggaagattcctggccccaaggcctcctccggct | |
| | cgctgattggcccagcggagagtgggcggggccggtgaagactccttaaa | |
| | ggcgcagggcggcgagcacggtcaccagacgctgacagctactcagaacc | |
| | aaatctggttccatccagagacaagcgaagacaagagaagcagagcagag | |
| | cggcgcgttcccgatcctcggccaggaccagccttccccagagcatccct | |
| | gccgcgggacgcaaccttcccaggagcatccctgccgcggagcaactttc | |
| | cccggagcatccagcccggacgcagcCTTCCAGAAGCACGAGCCCACCAC | |
| | TAGTGCCACC | |
| HSPm2 | AATTAGCTTGACCTGCAGCCTGAGGCAAAGGGAGTGGCTACAGCCTGGCA | SEQ ID NO: 11 |
| | CGGTCGATTAAGCCCTGCTCTCCGGGTCCTGGGACACTTTCCTTTTTCCT | |
| | CTTTTGAGTCACAGGTCCTCCTAACATGAGAATCAAGTATTTTCACGCTG | |
| | ATTTCCTTATAAAATTGTGAGAACTCCATAGGCGATGTACCGCCTACTCC | |
| | TACCTTAACCGTGATGTAAAGACAGCAAAACAAATGAACTATACTGCAAG | |
| | ATCTCTTCTATTTCCCTATTCAAACCTAAAATGAAGAGGGAGGGGGAGAC | |
| | ATGGACAAGCAAGCATTCCACAGGCGCCCCTGCCCAACGCTGTCACTCAA | |
| | ACCAGGACCCAATCACAGACTTTTTAGCCAAGCCTTATCCCGCCTCTCTT | |
| | GAGAAACTTTCTGCGTCCGCCATCCTGTAGGAAGGATTTGTACACTTTAA | |
| | ACTCCCTCCCTGGTCTGAGTCCCACACTCTCACCACCCAGCACCTTCAGG | |
| | AGCTGACCCTTAACAGCTTCACCCACAGGGACCCCGAAGTTGCGTCGCCT | |
| | CCGCAACAGTGTCAATAGCAGCACCAGCACTTCCCCACACCCTCCCCCTC | |
| | AGGAATCCGTACTCTCTAGCGAACCCCAGAAACCTCTGGAGAGTTCTGGA | |
| | CAAGGGCGGAACCCACAACTCCGATTACTCAAGGGAGGCGGGGAAGCTCC | |
| | ACCAGACGCGAAACTGCTGGAAGATTCCTGGCCCCAAGGCCTCCTCCGGC | |
| | TCGCTGATTGGCCCAGCGGAGAGTGGGCGGGGCCGGTGAAGACTCCTTAA | |
| | AGGCGCAGGGCGGCGAGCAGGGCACCAGACGCTGACAGCTACTCAGAATC | |
| | AAATCTGGTTCCATCCAGAGACAAGCGAAGACAAGAGAAGCAGAGCGAGC | |
| | GGCGCGTTCCCGATCCTCGGCCAGGACCAGCCTTCCCCAGAGCATCCACG | |
| | CCGCGGAGCGCAACCTTCCCAGGAGCATCCCTGCCGCGGAGCGCAACTTT | |
| | CCCCGGAGCATCCACGCCGCGGAGCGCAGCCTTCCAGAAGCAGAGCGCCA | |
| | CCACTAGTGCCACC | |
| HSPm3 | accagacgctgacagctactcagaaccaaatctggttccatccagagaca | SEQ ID NO: 12 |
| | agcgaagacaagagaagcagagcgagcggcgcgttcccgatcctcggcca | |
| | ggaccagccttccccagagcatccctgccgcggagcgcaaccttcccagg | |
| | agcatccctgccgcggagcgcaactttccccggagcatccacgccgcgga | |
| | gcgcagccttccagaagcagagcgcggcgcc | |
| HSP16F | gattgtagtttgaagatttcacaattagagtgaatgttgtttggttcggt | SEQ ID NO: 13 |
| | tttgtcactgtatttatactcatttccacctttttCTAGAAGGTCCTAGA | |
| | TGCATCTAGGACCTTCTAGAACATTCTAAacggctgcaggatacgggtat | |
| | ataagccaatcgtgttcagaggaaaccaatacactttgttcaagtgctta | |
| | ctgttcattctctaaacttcaagaCACC | |
| HSPmin | ACATTTCCTGTACAAGTGccCTAGGAGCTCGGATCCAGGAGGCCTAACTG | SEQ ID NO: 14 |
| | GCCGGTACCTGAGCTCCTGGAAGATTCTAGAACGTTCTGGAAGATTCTAG | |
| | AACGTTCCTCGAGGATATCAAGATCTGGCCTCGGCGGCCAAGCTTAGACA | |
| | CTAGAGGGTATATAATGGAAGCTCGACTTCCAGCTTGGCAATCCGGTACT | |
| | GTTGGTAAAGCgccacc | |

### Payloads

In some embodiments, the payload gene encodes a payload protein. The payload protein can comprise a factor locally down-regulating the activity of endogenous immune cells. The payload protein can be capable of remodeling a tumor microenvironment and/or reducing immunosuppression at a target site of a subject. The payload protein can comprise a degron. In some embodiments, the steady-state levels of the payload protein can be varied by varying the sequence of the degron. In some embodiments, the payload comprises a secreted protein. In some embodiments, induction of the first inducible promoter by thermal stimulation causes secretion of the payload molecule. In some embodiments, stimulated steady state payload protein levels, unstimulated steady state payload protein levels, the lower tuned threshold, and/or the upper tuned threshold can be tuned by adjusting the presence and/or sequence the tandem gene expression element. In some embodiments, the payload comprises a CAR and/or a TCR, wherein the payload is not expressed in the absence of thermal stimulus, and wherein engagement of the CAR and/or TCR initiates sustained expression of the payload. In some embodiments, the payload comprises a prodrug-converting enzyme (e.g., HSV thymidine kinase (TK), Cytosine Deaminase (CD), Purine nucleoside phosphorylase (PNP), Cytochrome p450 enzymes (CYP), Carboxypeptidases (CP), Caspase-9, Carboxylesterase (CE), Nitroreductase (NTR), Horse radish peroxidase (HRP), Guanine Ribosyltransferase (XGRTP), Glycosidase enzymes, Methionine-α,γ-lyase (MET), Thymidine phosphorylase (TP)).

In some embodiments, the payload gene encodes a payload RNA agent. A payload RNA agent can comprise one or more of dsRNA, siRNA, shRNA, pre-miRNA, pri-miRNA, miRNA, stRNA, lncRNA, piRNA, and snoRNA. In some embodiments, the payload gene encodes a siRNA, a shRNA, an antisense RNA oligonucleotide, an antisense miRNA, a trans-splicing RNA, a guide RNA, single-guide RNA, crRNA, a tracrRNA, a trans-splicing RNA, a pre-mRNA, a mRNA, or any combination thereof.

The payload protein can comprise a cytokine. The cytokine can be selected from the group consisting of interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, granulocyte macrophage colony stimulating factor (GM-CSF), M-CSF, SCF, TSLP, oncostatin M, leukemia-inhibitory factor (LIF), CNTF, Cardiotropin-1, NNT-1/BSF-3, growth hormone, Prolactin, Erythropoietin, Thrombopoietin, Leptin, G-CSF, or receptor or ligand thereof.

The payload protein can comprise a member of the TGF-β/BMP family selected from the group consisting of TGF-β1, TGF-β2, TGF-β3, BMP-2, BMP-3a, BMP-3b, BMP-4, BMP-5, BMP-6, BMP-7, BMP-8a, BMP-8b, BMP-9, BMP-10, BMP-11, BMP-15, BMP-16, endometrial bleeding associated factor (EBAF), growth differentiation factor-1 (GDF-1), GDF-2, GDF-3, GDF-5, GDF-6, GDF-7, GDF-8, GDF-9, GDF-12, GDF-14, mullerian inhibiting substance (MIS), activin-1, activin-2, activin-3, activin-4, and activin-5. The payload protein can comprise a member of the TNF family of cytokines selected from the group consisting of TNF-alpha, TNF-beta, LT-beta, CD40 ligand, Fas ligand, CD 27 ligand, CD 30 ligand, and 4-1 BBL. The payload protein can comprise a member of the immunoglobulin superfamily of cytokines selected from the group consisting of B7.1 (CD80) and B7.2 (B70). The payload protein can comprise an interferon. The interferon can be selected from interferon alpha, interferon beta, or interferon gamma. The payload protein can comprise a chemokine. The chemokine can be selected from CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13, or CXCL15. The payload protein can comprise a interleukin. The interleukin can be selected from IL-10 IL-12, IL-1, IL-6, IL-7, IL-15, IL-2, IL-18 or IL-21. The payload protein can comprise a tumor necrosis factor (TNF). The TNF can be selected from TNF-alpha, TNF-beta, TNF-gamma, CD252, CD154, CD178, CD70, CD153, or 4-1BBL.

The payload protein can comprise a CRE recombinase, GCaMP, a cell therapy component, a knock-down gene therapy component, a cell-surface exposed epitope, or any combination thereof. The payload protein can comprise a chimeric antigen receptor.

The payload protein can comprise a programmable nuclease. In some embodiments, the programmable nuclease is selected from the group comprising: SpCas9 or a derivative thereof, VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof, dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions. The programmable nuclease can comprise a zinc finger nuclease (ZFN) and/or transcription activator-like effector nuclease (TALEN). The programmable nuclease can comprise Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease, Cas1, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13c, derivatives thereof, or any combination thereof. The nucleic acid composition can comprise a polynucleotide encoding (i) a targeting molecule and/or (ii) a donor nucleic acid. The targeting molecule can be capable of associating with the programmable nuclease. The targeting molecule can comprise single strand DNA or single strand RNA. The targeting molecule can comprise a single guide RNA (sgRNA).

In some embodiments, the payload protein is a therapeutic protein or variant thereof. Non-limiting examples of therapeutic proteins include blood factors, such as β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-β), and the like; soluble receptors, such as soluble TNF-receptors, soluble VEGF receptors, soluble interleukin receptors (e.g., soluble IL-1 receptors and soluble type II IL-1 receptors), soluble γ/δ t cell receptors, ligand-binding fragments of a soluble receptor, and the like; enzymes, such as—glucosidase, imiglucarase, β-glucocerebrosidase, and alglucerase; enzyme activators, such as tissue plasminogen activator; chemokines, such as IP-10, monokine induced by interferon-gamma (Mig), Gro/IL-8, RANTES, MIP-l, MIP-I 3, MCP-1, PF-4, and the like; angiogenic agents, such as vascular endothelial growth factors (VEGFs, e.g., VEGF121, VEGF165, VEGF-C, VEGF-2), transforming growth factor-beta, basic fibroblast growth factor, glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); transforming growth factors (TGFs); tissue factors, luteinizing hormone; macrophage activating factors; tumor necrosis factor (TNF); neutrophil chemotactic factor (NCF); nerve growth factor; tissue inhibitors of metalloproteinases; vasoactive intestinal peptide; angiogenin; angiotropin; fibrin; hirudin; IL-1 receptor antagonists; and the like. Some other non-limiting examples of payload protein include ciliary neurotrophic factor (CNTF); brain-derived neurotrophic factor (BDNF); neurotrophins 3 and 4/5 (NT-3 and 4/5); glial cell derived neurotrophic factor (GDNF); aromatic amino acid decarboxylase (AADC); hemophilia related clotting proteins, such as Factor VIII, Factor IX, Factor X; dystrophin or mini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GLUT2), aldolase A, β-enolase, and glycogen synthase; lysosomal enzymes (e.g., beta-N-acetylhexosaminidase A); and any variants thereof.

In some embodiments, the payload protein is an active fragment of a protein, such as any of the aforementioned proteins. In some embodiments, the payload protein is a fusion protein comprising some or all of two or more proteins. In some embodiments a fusion protein can comprise all or a portion of any of the aforementioned proteins.

In some embodiments, the payload protein is a multi-subunit protein. For examples, the payload protein can comprise two or more subunits, or two or more independent polypeptide chains. In some embodiments, the payload protein can be an antibody. Examples of antibodies include, but are not limited to, antibodies of various isotypes (for example, IgG1, IgG2, IgG3, IgG4, IgA, IgD, IgE, and IgM); monoclonal antibodies produced by any means known to those skilled in the art, including an antigen-binding fragment of a monoclonal antibody; humanized antibodies; chimeric antibodies; single-chain antibodies; antibody fragments such as Fv, F(ab′)2, Fab′, Fab, Facb, scFv and the like; provided that the antibody is capable of binding to antigen. In some embodiments, the antibody is a full-length antibody.

In some embodiments, the payload gene encodes a pro-survival protein (e.g., Bcl-2, Bcl-XL, Mcl-1 and A1). In some embodiments, the payload gene encodes a apoptotic factor or apoptosis-related protein such as, for example, AIF, Apaf (e.g., Apaf-1, Apaf-2, and Apaf-3), oder APO-2 (L), APO-3 (L), Apopain, Bad, Bak, Bax, Bcl-2, Bcl-xL, Bcl-xs, bik, CAD, Calpain, Caspase (e.g., Caspase-1, Caspase-2, Caspase-3, Caspase-4, Caspase-5, Caspase-6, Caspase-7, Caspase-8, Caspase-9, Caspase-10, and Caspase-11), ced-3, ced-9, c-Jun, c-Myc, crm A, cytochrom C, CdR1, DcR1, DD, DED, DISC, DNA-PKcs, DR3, DR4, DR5, FADD/MORT-1, FAK, Fas (Fas-ligand CD95/fas (receptor)), FLICE/MACH, FLIP, fodrin, fos, G-Actin, Gas-2, gelsolin, granzyme A/B, ICAD, ICE, INK, Lamin A/B, MAP, MCL-1, Mdm-2, MEKK-1, MORT-1, NEDD, NF-ₖₐₚₚₐB, NuMa, p53, PAK-2, PARP, perforin, PITSLRE, PKCdelta, pRb, presenilin, prICE, RAIDD, Ras, RIP, sphingomyelinase, thymidinkinase from herpes simplex, TRADD, TRAF2, TRAIL-R1, TRAIL-R2, TRAIL-R3, and/or transglutaminase.

In some embodiments, the payload gene encodes a cellular reprogramming factor capable of converting an at least partially differentiated cell to a less differentiated cell, such as, for example, Oct-3, Oct-4, Sox2, c-Myc, Klf4, Nanog, Lin28, ASCL1, MYT1 L, TBX3b, SV40 large T, hTERT, miR-291, miR-294, miR-295, or any combinations thereof. In some embodiments, the payload gene encodes a programming factor that is capable of differentiating a given cell into a desired differentiated state, such as, for example, nerve growth factor (NGF), fibroblast growth factor (FGF), interleukin-6 (IL-6), bone morphogenic protein (BMP), neurogenin3 (Ngn3), pancreatic and duodenal homeobox 1 (Pdx1), Mafa, or any combination thereof.

In some embodiments, the payload gene encodes a human adjuvant protein capable of eliciting an innate immune response, such as, for example, cytokines which induce or enhance an innate immune response, including IL-2, IL-12, IL-15, IL-18, IL-21CCL21, GM-CSF and TNF-alpha; cytokines which are released from macrophages, including IL-1, IL-6, IL-8, IL-12 and TNF-alpha; from components of the complement system including C1q, MBL, C1r, C1s, C2b, Bb, D, MASP-1, MASP-2, C4b, C3b, C5a, C3a, C4a, C5b, C6, C7, C8, C9, CR1, CR2, CR3, CR4, C1qR, C1INH, C4 bp, MCP, DAF, H, I, P and CD59; from proteins which are components of the signaling networks of the pattern recognition receptors including TLR and IL-1 R1, whereas the components are ligands of the pattern recognition receptors including IL-1 alpha, IL-1 beta, Beta-defensin, heat shock proteins, such as HSP10, HSP60, HSP65, HSP70, HSP75 and HSP90, gp96, Fibrinogen, Typlll repeat extra domain A of fibronectin; the receptors, including IL-1 RI, TLR1, TLR2, TLR3, TLR4, TLR5, TLR6, TLR7, TLR8, TLR9, TLR10, TLR11; the signal transducers including components of the Small-GTPases signaling (e.g., RhoA, Ras, Rac1, Cdc42), components of the PIP signaling (e.g., PI3K, Src-Kinases), components of the MyD88-dependent signaling (e.g., MyD88, IRAK1, IRAK2), components of the MyD88-independent signaling (TICAM1, TICAM2 etc.); activated transcription factors including NF-κB, c-Fos, c-Jun, c-Myc; and induced target genes including IL-1 alpha, IL-1 beta, Beta-Defensin, IL-6, IFN gamma, IFN alpha and IFN beta; from costimulatory molecules, including CD28 or CD40-ligand or PD1; protein domains, including LAMP; cell surface proteins; or human adjuvant proteins including CD80, CD81, CD86, trif, flt-3 ligand, thymopentin, Gp96 or fibronectin, etc., or any species homolog of any of the above human adjuvant proteins.

As described herein, the nucleotide sequence encoding the payload protein can be modified to improve expression efficiency of the protein. The methods that can be used to improve the transcription and/or translation of a gene herein are not particularly limited. For example, the nucleotide sequence can be modified to better reflect host codon usage to increase gene expression (e.g., protein production) in the host (e.g., a mammal).

The degree of payload gene expression in the heat-inducible macrophages can vary. For example, in some embodiments, the payload gene encodes a payload protein. The amount of the payload protein expressed in the subject (e.g., the serum of the subject) can vary. For example, in some embodiments the protein can be expressed in the serum of the subject in the amount of at least about 9 μg/ml, at least about 10 μg/ml, at least about 50 μg/ml, at least about 100 μg/ml, at least about 200 μg/ml, at least about 300 μg/ml, at least about 400 μg/ml, at least about 500 μg/ml, at least about 600 μg/ml, at least about 700 μg/ml, at least about 800 μg/ml, at least about 900 μg/ml, or at least about 1000 μg/ml. In some embodiments, the payload protein is expressed in the serum of the subject in the amount of about 9 μg/ml, about 10 μg/ml, about 50 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1000 μg/ml, about 1500 μg/ml, about 2000 μg/ml, about 2500 μg/ml, or a range between any two of these values. A skilled artisan will understand that the expression level in which a payload protein is needed for the method to be effective can vary depending on non-limiting factors such as the particular payload protein and the subject receiving the treatment, and an effective amount of the protein can be readily determined by a skilled artisan using conventional methods known in the art without undue experimentation.

A payload protein encoded by a payload gene can be of various lengths. For example, the payload protein can be at least about 200 amino acids, at least about 250 amino acids, at least about 300 amino acids, at least about 350 amino acids, at least about 400 amino acids, at least about 450 amino acids, at least about 500 amino acids, at least about 550 amino acids, at least about 600 amino acids, at least about 650 amino acids, at least about 700 amino acids, at least about 750 amino acids, at least about 800 amino acids, or longer in length. In some embodiments, the payload protein is at least about 480 amino acids in length. In some embodiments, the payload protein is at least about 500 amino acids in length. In some embodiments, the payload protein is about 750 amino acids in length.

The payload genes can have different lengths in different implementations. The number of payload genes can be different in different embodiments. In some embodiments, the number of payload genes in a nucleic acid composition can be, or can be about, 1, 2, 3, 4, 5, 6, 7, 8, 9, 10, 11, 12, 13, 14, 15, 16, 17, 18, 19, 20, 21, 22, 23, 24, 25, or a number or a range between any two of these values. In some embodiments, the number of payload genes in a nucleic acid composition can be at least, or can be at most, 1, 2, 3, 4, 5, 6, 7, 8, 9, 10, 11, 12, 13, 14, 15, 16, 17, 18, 19, 20, 21, 22, 23, 24, 25. In some embodiments, a payload genes is, or is about, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, 10000, or a number or a range between any two of these values, nucleotides in length. In some embodiments, a payload gene is at least, or is at most, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, or 10000 nucleotides in length.

The payload can be an inducer of cell death. The payload can be induce cell death by a non-endogenous cell death pathway (e.g., a bacterial pore-forming toxin). In some embodiments, the payload can be a pro-survival protein. In some embodiments, the payload is a modulator of the immune system. The payload protein can comprise a CRE recombinase, GCaMP, a cell therapy component, a knock-down gene therapy component, a cell-surface exposed epitope, or any combination thereof.

A payload protein can comprise an agonistic or antagonistic antibody or antigen-binding fragment thereof specific to a checkpoint inhibitor or checkpoint stimulator molecule (e.g., PD1, PD-L1, PD-L2, CD27, CD28, CD40, CD137, OX40, GITR, ICOS, A2AR, B7-H3, B7-H4, BTLA, CTLA4, IDO, KIR, LAG3, PD-1, and/or TIM-3). The one or more payloads can comprise a secretion tag. The secretion tag can be selected from the group comprising AbnA, AmyE, AprE, BglC, BglS, Bpr, Csn, Epr, Ggt, GlpQ, HtrA, LipA, LytD, MntA, Mpr, NprE, OppA, PbpA, PbpX, Pel, PelB, PenP, PhoA, PhoB, PhoD, PstS, TasA, Vpr, WapA, WprA, XynA, XynD, YbdN, Ybxl, YcdH, YclQ, YdhF, YdhT, YfkN, YflE, YfmC, Yfnl, YhcR, YlqB, YncM, YnfF, YoaW, YocH, YolA, YqiX, Yqxl, YrpD, YrpE, YuaB, Yurl, YvcE, YvgO, YvpA, YwaD, YweA, YwoF, YwtD, YwtF, YxaLk, YxiA, and YxkC. A payload protein can comprise a constitutive signal peptide for protein degradation (e.g., PEST). A payload protein can comprise a nuclear localization signal (NLS) or a nuclear export signal (NES). A payload protein can comprise a dosage indicator protein. The dosage indicator protein can be detectable. The dosage indicator protein can comprise green fluorescent protein (GFP), enhanced green fluorescent protein (EGFP), yellow fluorescent protein (YFP), enhanced yellow fluorescent protein (EYFP), blue fluorescent protein (BFP), mScarlet, red fluorescent protein (RFP), TagRFP, Dronpa, Padron, mApple, mCherry, mruby3, rsCherry, rsCherryRev, derivatives thereof, or any combination thereof. A payload protein can comprise a degron. The steady-state levels of the payload protein can be varied by varying the sequence of the degron.

The payload RNA agent can comprise one or more of dsRNA, siRNA, shRNA, pre-miRNA, pri-miRNA, miRNA, stRNA, lncRNA, piRNA, and snoRNA. The first polynucleotide or the second polynucleotide can comprise a payload gene and one or more secondary payload gene(s). In some embodiments, said secondary payload gene(s) encode one or more secondary payload RNA agent(s) and/or one secondary payload protein(s). The payload gene and the one or more secondary payload gene(s) can be operably linked to a tandem gene expression element. The tandem gene expression element can be an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof. The payload transcript or the polycistronic transcript can be capable of being translated to generate: (i) a payload protein or payload RNA agent, and (ii) one or more secondary payload RNA agents and/or one or more secondary payload proteins.

A payload protein can be capable of diminishing the concentration, stability, and/or activity an endogenous protein. A payload protein can comprise a component of a synthetic protein circuit. The payload protein can be a therapeutic protein or a variant thereof (e.g., a therapeutic protein configured to prevent or treat a disease or disorder of a subject). In some embodiments, the subject suffers from a deficiency of said therapeutic protein. A payload protein can comprise fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof. A payload protein can comprise nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof. A payload protein can comprise a CRE recombinase, GCaMP, a cell therapy component, a knock-down gene therapy component, a cell-surface exposed epitope, or any combination thereof. A payload protein can comprise a diagnostic agent. The diagnostic agent can comprise green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCitrine, mCherry, mruby3, rsCherry, rsCherryRev, derivatives thereof, or any combination thereof. A payload protein can comprise a bispecific T cell engager (BiTE) (e.g., obinutuzumab, mosunetuzumab, selicrelumab, blinatumomab, ertumaxomab, maxomab, AMV564, AFM13, REGN-1979, GEN-3013, or pasotuxizumab).

Chimeric Antigen Receptors and Engineered t Cell Receptors

The payload protein can comprise a chimeric antigen receptor (CAR) or T-cell receptor (TCR). In some embodiments, the CAR comprises a T-cell receptor (TCR) antigen binding domain. The term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. The terms “CAR” and “CAR molecule” are used interchangeably. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some embodiments, the set of polypeptides are in the same polypeptide chain (e.g., comprise a chimeric fusion protein). In some aspects, the set of polypeptides are contiguous with each other. In some embodiments, the set of polypeptides are not contiguous with each other, e.g., are in different polypeptide chains. In some embodiments, the set of polypeptides include a dimerization switch that, upon the presence of a dimerization molecule, can couple the polypeptides to one another, e.g., can couple an antigen binding domain to an intracellular signaling domain. In some embodiments, the stimulatory molecule is the zeta chain associated with the t cell receptor complex. In some embodiments, the cytoplasmic signaling domain further comprises one or more functional signaling domains derived from at least one costimulatory molecule as defined below. In some embodiments, the costimulatory molecule is chosen from the costimulatory molecules described herein, e.g., 4-1BB (i.e., CD137), CD27 and/or CD28. In some embodiments, the CAR comprises a chimeric fusion protein comprising an extracellular antigen binding domain, a transmembrane domain and an intracellular signaling domain comprising a functional signaling domain derived from a stimulatory molecule. In some embodiments, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In some embodiments, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In some embodiments, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In some embodiments the CAR comprises an optional leader sequence at the amino-terminus (N-ter) of the CAR fusion protein. In some embodiments, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

The CAR and/or TCR can comprise one or more of an antigen binding domain, a transmembrane domain, and an intracellular signaling domain. The CAR or TCR further can comprise a leader peptide. The TCR further can comprise a constant region and/or CDR4. The term “signaling domain” refers to the functional portion of a protein which acts by transmitting information within the cell to regulate cellular activity via defined signaling pathways by generating second messengers or functioning as effectors by responding to such messengers. An “intracellular signaling domain,” as the term is used herein, refers to an intracellular portion of a molecule. The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines. In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a t cell receptor, and a costimulatory intracellular signaling domain can comprise cytoplasmic sequence from co-receptor or costimulatory molecule. A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12.

The intracellular signaling domain can comprise a primary signaling domain, a costimulatory domain, or both of a primary signaling domain and a costimulatory domain. The cytoplasmic domain or region of the CAR includes an intracellular signaling domain. An intracellular signaling domain is generally responsible for activation of at least one of the normal effector functions of the immune cell in which the CAR has been introduced. The term “effector function” refers to a specialized function of a cell. Effector function of a t cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The term a “costimulatory molecule” refers to a cognate binding partner on a t cell that specifically binds with a costimulatory ligand, thereby mediating a costimulatory response by the t cell, such as, but not limited to, proliferation. Costimulatory molecules are cell surface molecules other than antigen receptors or their ligands that are contribute to an efficient immune response. Costimulatory molecules include, but are not limited to an MHC class I molecule, BTLA and a Toll ligand receptor, as well as OX40, CD27, CD28, CD5, ICAM-1, LFA-1 (CD11a/CD18), ICOS (CD278), and 4-1BB (CD137). Further examples of such costimulatory molecules include CD5, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. The intracellular signaling domain can comprise the entire intracellular portion, or the entire native intracellular signaling domain, of the molecule from which it is derived, or a functional fragment or derivative thereof.

Examples of intracellular signaling domains for use in the CAR disclosed herein include the cytoplasmic sequences of the t cell receptor (TCR) and co-receptors that act in concert to initiate signal transduction following antigen receptor engagement, as well as any derivative or variant of these sequences and any recombinant sequence that has the same functional capability. It is known that signals generated through the TCR alone are insufficient for full activation of the t cell and that a secondary and/or costimulatory signal is also required. Thus, t cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain). A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs. The primary signaling domain can comprise a functional signaling domain of one or more proteins selected from the group consisting of CD3 zeta, CD3 gamma, CD3 delta, CD3 epsilon, common FcR gamma (FCERIG), FcR beta (Fc Epsilon Rib), CD79a, CD79b, Fcgamma RIIa, DAP10, and DAP12, or a functional variant thereof.

In some embodiments, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue. The costimulatory domain can comprise a functional domain of one or more proteins selected from the group consisting of CD27, CD28, 4-1BB (CD137), OX40, CD28-OX40, CD28-4-1BB, CD30, CD40, PD-1, ICOS, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D, or a functional variant thereof.

The portion of the CAR comprising an antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody, or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). The antigen binding domain of a CAR composition disclosed herein can comprises an antibody fragment. In some embodiments, the CAR comprises an antibody fragment that comprises a scFv.

In some embodiments, the CAR comprises a target-specific binding element otherwise referred to as an antigen binding domain. The choice of moiety depends upon the type and number of ligands that define the surface of a target cell. For example, the antigen binding domain may be chosen to recognize a ligand that acts as a cell surface marker on target cells associated with a particular disease state. Thus, examples of cell surface markers that may act as ligands for the antigen binding domain in a CAR include those associated with viral, bacterial and parasitic infections, autoimmune disease and cancer cells.

In some embodiments, the CAR-mediated T-cell response can be directed to an antigen of interest by way of engineering an antigen binding domain that specifically binds a desired antigen into the CAR. In some embodiments, the portion of the CAR comprising the antigen binding domain comprises an antigen binding domain that targets a tumor antigen, e.g., a tumor antigen described herein. The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a t cell receptor (TCR), or a fragment there of, e.g., single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment. In some embodiments, the antigen binding domain comprises a humanized antibody or an antibody fragment. The non-human antibody can be humanized, where specific sequences or regions of the antibody are modified to increase similarity to an antibody naturally produced in a human or fragment thereof. In some embodiments, the antigen binding domain is humanized.

The antigen binding domain can comprise an antibody, an antibody fragment, an scFv, a Fv, a Fab, a (Fab′)2, a single domain antibody (SDAB), a VH or VL domain, a camelid VHH domain, a Fab, a Fab′, a F(ab′)2, a Fv, a scFv, a dsFv, a diabody, a triabody, a tetrabody, a multispecific antibody formed from antibody fragments, a single-domain antibody (sdAb), a single chain comprising cantiomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof.

In some embodiments, the antigen binding domain is a t cell receptor (“TCR”), or a fragment thereof, for example, a single chain TCR (scTCR). Methods to make such TCRs are known in the art. See, e.g., Willemsen R A et al, Gene Therapy 7: 1369-1377 (2000); Zhang T et al, Cancer Gene Ther 11: 487-496 (2004); Aggen et al, Gene Ther. 19(4):365-74 (2012) (references are incorporated herein by its entirety). For example, scTCR can be engineered that contains the Vα and Vβ genes from a t cell clone linked by a linker (e.g., a flexible peptide). This approach is very useful to cancer associated target that itself is intracellar, however, a fragment of such antigen (peptide) is presented on the surface of the cancer cells by MHC.

In some embodiments, the antigen binding domain is a multispecific antibody molecule. In some embodiments, the multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope.

The antigen binding domain can be configured to bind to a tumor antigen. The terms “cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CARs includes CARs comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by t cell receptors (TCRs) on CD8+T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

The tumor antigen can be a solid tumor antigen. The tumor antigen can be selected from the group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCTA-1 or Galectin 8), melanoma antigen recognized by t cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By t cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

The tumor antigen can be selected from the group comprising CD150, 5T4, ActRIIA, B7, BMCA, CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRvIII, EGP-2, EGP-4, EPHa2, ErbB2, ErbB3, ErbB4, FBP, GD2, GD3, HER1-HER2 in combination, HER2-HER3 in combination, HERV-K, HIV-1 envelope glycoprotein gp120, HIV-1 envelope glycoprotein gp41, HLA-DR, HM1.24, HMW-MAA, Her2, Her2/neu, IGF-1R, IL-11Ralpha, IL-13R-alpha2, IL-2, IL-22R-alpha, IL-6, IL-6R, Ia, Ii, L1-CAM, L1-cell adhesion molecule, Lewis Y, L1-CAM, MAGE A3, MAGE-A1, MART-1, MUC1, NKG2C ligands, NKG2D Ligands, NY-ESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-1, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D1), ephrinB2, epithelial tumor antigen, estrogen receptor, fetal acethycholine e receptor, folate binding protein, gp100, hepatitis B surface antigen, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, β2-Microglobulin, Fc Receptor-like 5 (FcRL5), or molecules expressed by HIV, HCV, HBV, or other pathogens.

The antigen binding domain can be connected to the transmembrane domain by a hinge region. In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge (e.g., an IgG4 hinge, an IgD hinge), a GS linker (e.g., a GS linker described herein), a KIR2DS2 hinge or a CD8a hinge.

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In some embodiments, the transmembrane domain is one that is associated with one of the other domains of the CAR e.g., in one embodiment, the transmembrane domain may be from the same protein that the signaling domain, costimulatory domain or the hinge domain is derived from. In some embodiments, the transmembrane domain is not derived from the same protein that any other domain of the CAR is derived from. In some instances, the transmembrane domain can be selected or modified by amino acid substitution to avoid binding of such domains to the transmembrane domains of the same or different surface membrane proteins, e.g., to minimize interactions with other members of the receptor complex. In some embodiments, the transmembrane domain is capable of homodimerization with another CAR on the cell surface of a CAR-expressing cell. In some embodiments, the amino acid sequence of the transmembrane domain may be modified or substituted so as to minimize interactions with the binding domains of the native binding partner present in the same CAR-expressing cell.

The transmembrane domain can comprise a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7Ra, ITGA1, VLA1, CD49a, ITGA4, IA4, CD49D, ITGA6, VLA-6, CD49f, ITGAD, CD11 d, ITGAE, CD103, ITGAL, CD11a, LFA-1, ITGAM, CD11 b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C, or a functional variant thereof. The transmembrane domain may be derived either from a natural or from a recombinant source. Where the source is natural, the domain may be derived from any membrane-bound or transmembrane protein. In some embodiments the transmembrane domain is capable of signaling to the intracellular domain(s) whenever the CAR has bound to a target.

Receptors & Targeting Moieties

The macrophages can comprise one or more receptors and/or targeting moieties configured to bind a component of a target site of a subject. Said one or more receptors and/or targeting moieties can be payload protein(s). The one or more receptors and/or the one or more targeting moieties can be selected from the group comprising mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, and an RGD peptide or RGD peptide mimetic. The one or more targeting moieties and/or one or more receptors can comprise one or more of the following: an antibody or antigen-binding fragment thereof, a peptide, a polypeptide, an enzyme, a peptidomimetic, a glycoprotein, a lectin, a nucleic acid, a monosaccharide, a disaccharide, a trisaccharide, an oligosaccharide, a polysaccharide, a glycosaminoglycan, a lipopolysaccharide, a lipid, a vitamin, a steroid, a hormone, a cofactor, a receptor, a receptor ligand, and analogs and derivatives thereof. The antibody or antigen-binding fragment thereof can comprise a Fab, a Fab′, a F(ab′)2, a Fv, a scFv, a dsFv, a diabody, a triabody, a tetrabody, a multispecific antibody formed from antibody fragments, a single-domain antibody (sdAb), a single chain comprising complementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof. The one or more targeting moieties and/or one or more receptors can be configured to bind one or more of the following: CD3, CD4, CD5, CD6, CD7, CD8, CD9, CD10, CD11a, CD11b, CD11c, CD12w, CD14, CD15, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD46, CD47, CD48, CD49b, CD49c, CD51, CD52, CD53, CD54, CD55, CD56, CD58, CD59, CD61, CD62E, CD62L, CD62P, CD63, CD66, CD68, CD69, CD70, CD72, CD74, CD79, CD79a, CD79b, CD80, CD81, CD82, CD83, CD86, CD87, CD88, CD89, CD90, CD91, CD95, CD96, CD98, CD100, CD103, CD105, CD106, CD109, CD117, CD120, CD125, CD126, CD127, CD133, CD134, CD135, CD137, CD138, CD141, CD142, CD143, CD144, CD147, CD151, CD147, CD152, CD154, CD156, CD158, CD163, CD166, CD168, CD174, CD180, CD184, CDw186, CD194, CD195, CD200, CD200a, CD200b, CD209, CD221, CD227, CD235a, CD240, CD262, CD271, CD274, CD276 (B7-H3), CD303, CD304, CD309, CD326, 4-1BB, 5 AC, 5T4 (Trophoblast glycoprotein, TPBG, 5T4, Wnt-Activated Inhibitory Factor 1 or WAIF1), Adenocarcinoma antigen, AGS-5, AGS-22M6, Activin receptor like kinase 1, AFP, AKAP-4, ALK, Alpha integrin, Alpha v beta6, Amino-peptidase N, Amyloid beta, Androgen receptor, Angiopoietin 2, Angiopoietin 3, Annexin A1, Anthrax toxin protective antigen, Anti-transferrin receptor, AOC3 (VAP-1), B7-H3, Bacillus anthracis anthrax, BAFF (B-cell activating factor), B-lymphoma cell, bcr-abl, Bombesin, BORIS, C5, C242 antigen, CA125 (carbohydrate antigen 125, MUC16), CA-IX (CAIX, carbonic anhydrase 9), CALLA, CanAg, Canis lupus familiaris IL31, Carbonic anhydrase IX, Cardiac myosin, CCL11 (C-C motif chemokine 11), CCR4 (C-C chemokine receptor type 4, CD194), CCR5, CD3E (epsilon), CEA (Carcinoembryonic antigen), CEACAM3, CEACAM5 (carcinoembryonic antigen), CFD (Factor D), Ch4D5, Cholecystokinin 2 (CCK2R), CLDN18 (Claudin-18), Clumping factor A, CRIPTO, FCSF1R (Colony stimulating factor 1 receptor, CD 115), CSF2 (colony stimulating factor 2, Granulocyte-macrophage colony-stimulating factor (GM-CSF)), CTLA4 (cytotoxic T-lymphocyte-associated protein 4), CTAA16.88 tumor antigen, CXCR4 (CD 184), C-X-C chemokine receptor type 4, cyclic ADP ribose hydrolase, Cyclin B 1, CYP1B 1, Cytomegalovirus, Cytomegalovirus glycoprotein B, Dabigatran, DLL4 (delta-like-ligand 4), DPP4 (Dipeptidyl-peptidase 4), DR5 (Death receptor 5), E. coli Shiga toxin type-1, E. coli Shiga toxin type-2, ED-B, EGFL7 (EGF-like domain-containing protein 7), EGFR, EGFRII, EGFRvIII, Endoglin (CD 105), Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, FOLR (folate receptor), Folate receptor alpha, Folate hydrolase, Fos-related antigen 1.F protein of respiratory syncytial virus, Frizzled receptor, Fucosyl GM1, GD2 ganglioside, G-28 (a cell surface antigen glycolipid), GD3 idiotype, GloboH, Glypican 3, N-glycolylneuraminic acid, GM3, GMCSF receptor a-chain, Growth differentiation factor 8, GP100, GPNMB (Transmembrane glycoprotein NMB), GUCY2C (Guanylate cyclase 2C, guanylyl cyclase C (GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-γ, Influenza hemagglutinin, IgE, IgE Fc region, IGHE, IL-1, IL-2 receptor (interleukin 2 receptor), IL-4, IL-5, IL-6, IL-6R (interleukin 6 receptor), IL-9, IL-10, IL-12, IL-13, IL-17, IL-17A, IL-20, IL-22, IL-23, IL31RA, ILGF2 (Insulin-like growth factor 2), Integrins (α4, αᵤβ₃, αvβ3, α₄β₇, α5β1, α6β4, α7β7, α11β3, α5β5, αvβ5), Interferon gamma-induced protein, ITGA2, ITGB2, KIR2D, LCK, Le, Legumain, Lewis-Y antigen, LFA-1 (Lymphocyte function-associated antigen 1, CD11a), LHRH, LINGO-1, Lipoteichoic acid, LIV1A, LMP2, LTA, MAD-CT-1, MAD-CT-2, MAGE-1, MAGE-2, MAGE-3, MAGE A1, MAGE A3, MAGE 4, MARTI, MCP-1, MIF (Macrophage migration inhibitory factor, or glycosylation inhibiting factor (GIF)), MS4A1 (membrane-spanning 4-domains subfamily A member 1), MSLN (mesothelin), MUCl (Mucin 1, cell surface associated (MUC1) or polymorphic epithelial mucin (PEM)), MUC1-KLH, MUC16 (CA125), MCPl (monocyte chemotactic protein 1), MelanA/MARTl, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES 1, P21, p53 nonmutant, P97, Page 4, PAP, Paratope of anti-(N-glycolylneuraminic acid), PAX3, PAX5, PCSK9, PDCD1 (PD-1, Programmed cell death protein 1, CD279), PDGF-Ra (Alpha-type platelet-derived growth factor receptor), PDGFR-β, PDL-1, PLAC1, PLAP-like testicular alkaline phosphatase, Platelet-derived growth factor receptor beta, Phosphate-sodium co-transporter, PMEL 17, Polysialic acid, Proteinase3 (PR1), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI), CD240), Rhesus factor, RANKL, RhoC, Ras mutant, RGS5, ROBO4, Respiratory syncytial virus, RON, Sarcoma translocation breakpoints, SART3, Sclerostin, SLAMF7 (SLAM family member 7), Selectin P, SDC1 (Syndecan 1), sLe(a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-a, TGF-β (Transforming growth factor beta), TGF-β1, TGF-β2 (Transforming growth factor-beta 2), Tie (CD202b), Tie2, TIM-1 (CDX-014), Tn, TNF, TNF-a, TNFRSF8, TNFRSF10B (tumor necrosis factor receptor superfamily member 10), TNFRSF13B (tumor necrosis factor receptor superfamily member 13B), TPBG (trophoblast glycoprotein), TRAIL-R1 (Tumor necrosis apoptosis Inducing ligand Receptor 1), TRAILR2 (Death receptor 5 (DR5)), tumor-associated calcium signal transducer 2, tumor specific glycosylation of MUC1, TWEAK receptor, TYRP1 (glycoprotein 75), TRP-2, Tyrosinase, VCAM-1 (CD 106), VEGF, VEGF-A, VEGF-2 (CD309), VEGFR-1, VEGFR2, or vimentin, WT1, XAGE 1, or cells expressing any insulin growth factor receptors, or any epidermal growth factor receptors. In some embodiments, the macrophages do not comprise an exogenous receptor or targeting moiety configured to bind a component of a target site of a subject. The macrophages can be capable of trafficking to solid tumor(s) of a subject via endogenous physiological signals and/or endogenous receptors.

### Methods of Treating a Disease or Disorder

Disclosed herein include methods of treating a disease or disorder in a subject. In some embodiments, the method comprises: administering to the subject an effective amount of the heat-inducible macrophages disclosed herein.

The subject can be a mammal. In some embodiments, the disease is associated with expression of a tumor antigen, wherein the disease associated with expression of a tumor antigen is selected from the group consisting of a proliferative disease, a precancerous condition, a cancer, and a non-cancer related indication associated with expression of the tumor antigen. The disease or disorder can be a cancer (e.g., a solid tumor). The cancer can be selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers.

The cancer can be a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

In some embodiments, the method comprises: administering an effective amount of pro-death agent and/or a transactivator-binding compound to the subject prior to, during, and/or after administration of the heat-inducible macrophages. The amount of transactivator-binding compound can be an amount effective to induce or attenuate a sufficient level of payload expression to treat the subject. In some embodiments, the transactivator-binding compound comprises tetracycline, doxycycline or a derivative thereof.

Administering can comprise aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof. The heat-inducible macrophages can be administered at a therapeutically effective amount. For example, a therapeutically effective amount of the heat-inducible macrophages can be at least about 10⁴ cells, at least about 10⁵ cells, at least about 10⁶ cells, at least about 10⁷ cells, at least about 10⁸ cells, at least about 10⁹, or at least about 10¹⁰. In another embodiment, the therapeutically effective amount of the heat-inducible macrophages is about 10⁴ cells, about 10⁵ cells, about 10⁶ cells, about 10⁷ cells, or about 10⁸ cells. In one particular embodiment, the therapeutically effective amount of the heat-inducible macrophages is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg.

The heat-inducible macrophages described herein may be included in a composition for therapy. In some embodiments, the composition comprises a population of heat-inducible macrophages. The composition may include a pharmaceutical composition and further include a pharmaceutically acceptable carrier. A therapeutically effective amount of the pharmaceutical composition comprising the heat-inducible macrophages may be administered. The heat-inducible macrophages may be administered either alone, or as a pharmaceutical composition in combination with diluents and/or with other components such as IL-2 or other cytokines or cell populations. Ex vivo procedures are well known in the art. Briefly, cells are isolated from a mammal (e.g., a human) and genetically modified (i.e., transduced or transfected in vitro) with a nucleic acid composition (e.g., a vector) disclosed herein or a composition disclosed herein, thereby generating one or more heat-inducible macrophages. The heat-inducible macrophages can be administered to a mammalian recipient to provide a therapeutic benefit. The mammalian recipient may be a human and the heat-inducible macrophages can be autologous with respect to the recipient. Alternatively, the heat-inducible macrophages can be allogeneic, syngeneic or xenogeneic with respect to the recipient.

Applying Thermal Energy

In some embodiments, the method comprises: applying thermal energy to a target site of the subject sufficient to increase the local temperature of the target site to an activating temperature, thereby inducing the expression of the payload in heat-inducible macrophages at the target site. The activating temperature can be about 37.5° C., about 38.0° C., about 38.5° C., about 39.0° C., about 39.5° C., about 40.0° C., about 40.5° C., about 41.0° C., about 41.5° C., about 42.0° C., about 42.5° C., about 43.0° C., about 43.5° C., about 44.0° C., about 44.5° C., about 45.0° C., about 45.5° C., or about 46.0° C., or a number or a range between any two of these values.

Applying thermal energy to a target site of the subject can comprise the application of one or more of focused ultrasound (FUS), magnetic hyperthermia, microwaves, infrared irradiation, liquid-based heating, and contact heating. Liquid-based heating can comprise intraperitoneal chemotherapy (HIPEC). The term “applying ultrasound” shall be given its ordinary meaning, and shall also refer to sending ultrasound-range acoustic energy to a target. The sound energy produced by the piezoelectric transducer can be focused by beamforming, through transducer shape, lensing, or use of control pulses. The soundwave formed is transmitted to the body, then partially reflected or scattered by structures within a body; larger structures typically reflecting, and smaller structures typically scattering. The return sound energy reflected/scattered to the transducer vibrates the transducer and turns the return sound energy into electrical signals to be analyzed for imaging. The frequency and pressure of the input sound energy can be controlled and are selected based on the needs of the particular imaging/delivery task

The period of time between the administering and applying thermal energy can be about 48 hours, about 44 hours, about 40 hours, about 35 hours, about 30 hours, about 25 hours, 20 hours, 15 hours, 10 hours, about 8 hours, about 8 hours, 8 hours, about 7 hours, about 6 hours, about 5 hours, about 4 hours, about 3 hours, about 2 hours, about 1 hour, about 30 minutes, about 15 minutes, about 10 minutes, about 5 minutes, or a number or a range between any two of these values.

Applying thermal energy to a target site can comprise a continuous application of thermal energy to the target site over a duration of time. Applying thermal energy to a target site can comprise applying one or more pulses of thermal energy to the target site over a duration of time. The duration of time can be about 48 hours, about 44 hours, about 40 hours, about 35 hours, about 30 hours, about 25 hours, 20 hours, 15 hours, 10 hours, about 8 hours, about 8 hours, 8 hours, about 7 hours, about 6 hours, about 5 hours, about 4 hours, about 3 hours, about 2 hours, about 1 hour, about 30 minutes, about 15 minutes, about 10 minutes, about 5 minutes, or a number or a range between any two of these values.

In some embodiments, the one or more pulses have a duty cycle of greater than about 1% and less than about 100%. The one or more pulses have a duty cycle of about 0.000000001%, 0.00000001%, 0.0000001%, 0.000001%, 0.00001%, 0.0001%, 0.001%, 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values.

In some embodiments, the one or more pulses each have a pulse duration of about 1 hour, about 30 minutes, about 15 minutes, about 10 minutes, or about 5 minutes, about 1 minute, about 1 second, about 1 millisecond, or a number or a range between any two of these values.

In some embodiments, the method comprises: monitoring the temperature of the target region. The monitoring can be performed by magnetic resonance imaging (MRI) and/or fiber optic thermometry. The application of thermal energy to a target site of the subject can be guided spatially by magnetic resonance imaging (MRI).

Target Sites

The target site can comprise a solid tumor. The target site can comprise a site of disease or disorder or can be proximate to a site of a disease or disorder. The location of the one or more sites of a disease or disorder can be predetermined, can be determined during the method, or both. The target site can be an immunosuppressive environment. The target site can comprise a tissue. The tissue can be inflamed tissue and/or infected tissue. The tissue can comprise adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue and/or fat tissue. The tissue can comprise: (i) grade I, grade II, grade III or grade IV cancerous tissue; (ii) metastatic cancerous tissue; (iii) mixed grade cancerous tissue; (iv) a sub-grade cancerous tissue; (v) healthy or normal tissue; and/or (vi) cancerous or abnormal tissue. In some embodiments, at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or a number or a range between any two of these values, of the heat-inducible macrophages at the target site express the payload protein after applying thermal energy to the target site. In some embodiments, less than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or a number or a range between any two of these values, of the heat-inducible macrophages at a site other than the target site express the payload protein.

The ratio of the concentration of payload-expressing heat-inducible macrophages at the subject's target site to the concentration of payload-expressing heat-inducible macrophages in subject's blood, serum, or plasma can be vary. The ratio of the concentration of payload-expressing heat-inducible macrophages at the subject's target site to the concentration of payload-expressing heat-inducible macrophages in subject's blood, serum, or plasma can be, or be about, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, 10000:1, or a number or a range between any two of the values. In some embodiments, the ratio of the concentration of payload-expressing heat-inducible macrophages at the subject's target site to the concentration of payload-expressing heat-inducible macrophages in subject's blood, serum, or plasma can be at least, or be at most, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, or 10000:1.

The ratio of the concentration of payload protein at the subject's target site to the concentration of payload protein in subject's blood, serum, or plasma can be vary. In some embodiments, the ratio of the concentration of payload protein at the subject's target site to the concentration of payload protein in subject's blood, serum, or plasma can be, or be about, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, 10000:1, or a number or a range between any two of the values. In some embodiments, the ratio of the concentration of payload protein at the subject's target site to the concentration of payload protein in subject's blood, serum, or plasma can be at least, or be at most, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, or 10000:1.

The target site can comprise target cells. The target cells can be tumor cells (e.g., solid tumor cells). In some embodiments, the application of thermal energy to a target site of the subject results in the death of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or a number or a range between any two of these values, of the target cells. Non-target cells can comprise cells of the subject other than target cells. The ratio of target cell death to non-target cell death after application of thermal energy can be at least about 2:1 In some embodiments, the ratio of target cell death to non-target cell death after application of thermal energy can be, or be about, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, 10000:1, or a number or a range between any two of the values. In some embodiments, the ratio of target cell death to non-target cell death after application of thermal energy can be at least, or be at most, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, or 10000:1. The ratio of target cell death to non-target cell death can be at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, or a number or a range between any of these values) greater as compared to a method comprising macrophages constitutively expressing the payload protein. In some embodiments, the ratio of the concentration of payload protein at the subject's target site to the concentration of payload protein in a non-target site of the subject is about 2:1 to about 3000:1, about 2:1 to about 2000:1, about 2:1 to about 1000:1, or about 2:1 to about 600:1. Said non-target sites can comprise the lungs and/or liver.

In some embodiments, upon administration, the macrophages migrate to tumor(s) of the subject. In some embodiments, at least about 20 percent of the population of macrophages migrate to tumor(s) of the subject. In some embodiments, said macrophages constitute at least about 10 percent of the volume of said tumor(s) about 7 days post-administration. In some embodiments, the administration of the population of macrophages does not cause neurological side effects or the development of type 1 diabetes mellitus, hypothyroidism, adrenocortical hormone insufficiency, and/or other autoimmune-like syndromes. In some embodiments, at least about 20 percent of the population of macrophages present in the target site of the subject have undergone the recombination event about four days after applying thermal energy to the target site. Administering to the subject an effective amount of the population of heat-inducible macrophages can comprise two or more administrations. Applying thermal energy to a target site of the subject can comprise applying thermal energy to two or more target site(s) of the subject. The method can comprise: prior to the administering step, transducing macrophages with a viral vector comprising a circuit disclosed herein (e.g., a lentiviral vector) to generate an effective amount of the population of heat-inducible macrophages. The method can comprise selecting heat-inducible macrophages that have non undergone the recombination event. In some embodiments, heat-inducible macrophages that have non undergone the recombination event are detected via the presence of the detectable protein. In some embodiments, the expression of the payload from the heat-inducible macrophages is not suppressed by the tumor microenvironment of solid tumor(s). At least about 20 percent of the population of heat-inducible macrophages can be viable 7 days post-administration.

Additional Agents

The method can comprise administering one or more additional agents to the subject. In some embodiments, the one or more additional agents increases the efficacy of the heat-inducible macrophages. The one or more additional agents can comprise a protein phosphatase inhibitor, a kinase inhibitor, a cytokine, an inhibitor of an immune inhibitory molecule, or any combination thereof. The one or more additional agents can comprise an immune modulator, an anti-metastatic, a chemotherapeutic, a hormone or a growth factor antagonist, an alkylating agent, a TLR agonist, a cytokine antagonist, a cytokine antagonist, or any combination thereof. The one or more additional agents can comprise an agonistic or antagonistic antibody specific to a checkpoint inhibitor or checkpoint stimulator molecule such as PD1, PD-L1, PD-L2, CD27, CD28, CD40, CD137, OX40, GITR, ICOS, A2AR, B7-H3, B7-H4, BTLA, CTLA4, IDO, KIR, LAG3, PD-1, TIM-3.

The one or more additional agents can be selected from the group consisting of alkylating agents (nitrogen mustards, ethylenimine derivatives, alkyl sulfonates, nitrosoureas and triazenes); uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®); bendamustine (Treakisym®, Ribomustin®, Treanda®); chlormethine (Mustargen®); cyclophosphamide (Cytoxan®, Neosar®, Clafen®, Endoxan®, Procytox®, Revimmune™); ifosfamide (Mitoxana®); melphalan (Alkeran®); Chlorambucil (Leukeran®); pipobroman (Amedel®, Vercyte®); triethylenemelamine (Hemel®, Hexylen®, Hexastat®); triethylenethiophosphoramine; Temozolomide (Temodar®); thiotepa (Thioplex®); busulfan (Busilvex®, Myleran®); carmustine (BiCNU®); lomustine (CeeNU®); streptozocin (Zanosar®); estramustine (Emcyt®, Estracit®); fotemustine; irofulven; mannosulfan; mitobronitol; nimustine; procarbazine; ranimustine; semustine; triaziquone; treosulfan; and Dacarbazine (DTIC-Dome®); anti-EGFR antibodies (e.g., cetuximab (Erbitux®), panitumumab (Vectibix®), and gefitinib (Iressa®)); anti-Her-2 antibodies (e.g., trastuzumab (Herceptin®) and other antibodies from Genentech); antimetabolites (including, without limitation, folic acid antagonists (also referred to herein as antifolates), pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), carmofur, cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), mercaptopurine (Puri-Nethol®), capecitabine (Xeloda®), nelarabine (Arranon®), azacitidine (Vidaza®), decitabine (Dacogen®), enocitabine (Sunrabin®), sapacitabine, tegafur-uracil, tiazofurine, tioguanine, trofosfamide, and gemcitabine (Gemzar®); vinca alkaloids: vinblastine (Velban®, Velsar®), vincristine (Vincasar®, Oncovin®), vindesine (Eldisine®), vinorelbine (Navelbine®), vinflunine (Javlor®); platinum-based agents: carboplatin (Paraplat®, Paraplatin®), cisplatin (Platinol®), oxaliplatin (Eloxatin®), nedaplatin, satraplatin, and triplatin; anthracyclines: daunorubicin (Cerubidine®, Rubidomycin®), doxorubicin (Adriamycin®), epirubicin (Ellence®), idarubicin (Idamycin®), mitoxantrone (Novantrone®), valrubicin (Valstar®), aclarubicin, amrubicin, liposomal doxorubicin, liposomal daunorubicin, pirarubicin, pixantrone, and zorubicin; topoisomerase inhibitors: topotecan (Hycamtin®), irinotecan (Camptosar®), etoposide (Toposar®, VePesid®), teniposide (Vumon®), lamellarin D, SN-38, camptothecin (e.g., IT-101), belotecan, and rubitecan; taxanes: paclitaxel (Taxol®), docetaxel (Taxotere®), larotaxel, cabazitaxel, ortataxel, and tesetaxel; antibiotics: actinomycin (Cosmegen®), bleomycin (Blenoxane®), hydroxyurea (Droxia®, Hydrea®), mitomycin (Mitozytrex®, Mutamycin®); immunomodulators: lenalidomide (Revlimid®), thalidomide (Thalomid®); immune cell antibodies: alemtuzamab (Campath®), gemtuzumab (Myelotarg®), rituximab (Rituxan®), tositumomab (Bexxar®); interferons (e.g., IFN-alpha (Alferon®, Roferon-A®, Intron®-A) or IFN-gamma (Actimmune®)); interleukins: IL-1, IL-2 (Proleukin®), IL-24, IL-6 (Sigosix®), IL-12; HSP90 inhibitors (e.g., geldanamycin or any of its derivatives). In some embodiments, the HSP90 inhibitor is selected from geldanamycin, 17-alkylamino-17-desmethoxygeldanamycin (“17-AAG”) or 17-(2-dimethylaminoethyl)amino-17-desmethoxygeldanamycin (“17-DMAG”); anti-androgens which include, without limitation nilutamide (Nilandron®) and bicalutamide (Caxodex®); antiestrogens which include, without limitation tamoxifen (Nolvadex®), toremifene (Fareston®), letrozole (Femara®), testolactone (Teslac®), anastrozole (Arimidex®), bicalutamide (Casodex®), exemestane (Aromasin®), flutamide (Eulexin®), fulvestrant (Faslodex®), raloxifene (Evista®, Keoxifene®) and raloxifene hydrochloride; anti-hypercalcaemia agents which include without limitation gallium (III) nitrate hydrate (Ganite®) and pamidronate disodium (Aredia®); apoptosis inducers which include without limitation ethanol, 2-[[3-(2,3-dichlorophenoxy)propyl]amino]-(9Cl), gambogic acid, elesclomol, embelin and arsenic trioxide (Trisenox®); Aurora kinase inhibitors which include without limitation binucleine 2; Bruton's tyrosine kinase inhibitors which include without limitation terreic acid; calcineurin inhibitors which include without limitation cypermethrin, deltamethrin, fenvalerate and tyrphostin 8; CaM kinase II inhibitors which include without limitation 5-Isoquinolinesulfonic acid, 4-[{2S)-2-[(5-isoquinolinylsulfonyl)methylamino]-3-oxo-3-{4-phenyl-1-piperazinyl)propyl]phenyl ester and benzenesulfonamide; CD45 tyrosine phosphatase inhibitors which include without limitation phosphonic acid; CDC25 phosphatase inhibitors which include without limitation 1,4-naphthalene dione, 2,3-bis[(2-hydroxyethyl)thio]-(9Cl); CHK kinase inhibitors which include without limitation debromohymenialdisine; cyclooxygenase inhibitors which include without limitation 1H-indole-3-acetamide, 1-(4-chlorobenzoyl)-5-methoxy-2-methyl-N-(2-phenylethyl)-(9C1), 5-alkyl substituted 2-arylaminophenylacetic acid and its derivatives (e.g., celecoxib (Celebrex®), rofecoxib (Vioxx®), etoricoxib (Arcoxia®), lumiracoxib (Prexige®), valdecoxib (Bextra®) or 5-alkyl-2-arylaminophenylacetic acid); cRAF kinase inhibitors which include without limitation 3-(3,5-dibromo-4-hydroxybenzylidene)-5-iodo-1,3-dihydroindol-2-one and benzamide, 3-(dimethylamino)-N-[3-[(4-hydroxybenzoyl)amino]-4-methylphenyl]-(9C1); cyclin dependent kinase inhibitors which include without limitation olomoucine and its derivatives, purvalanol B, roascovitine (Seliciclib®), indirubin, kenpaullone, purvalanol A and indirubin-3′-monooxime; cysteine protease inhibitors which include without limitation 4-morpholinecarboxamide, N-[(1S)-3-fluoro-2-oxo-1-(2-phenylethyl)propyl]amino]-2-oxo-1-(phenylmethyl)ethyl]-(9Cl); DNA intercalators which include without limitation plicamycin (Mithracin®) and daptomycin (Cubicin®); DNA strand breakers which include without limitation bleomycin (Blenoxane®); E3 ligase inhibitors which include without limitation N-((3,3,3-trifluoro-2-trifluoromethyl)propionyl)sulfanilamide; EGF Pathway Inhibitors which include, without limitation tyrphostin 46, EKB-569, erlotinib (Tarceva®), gefitinib (Iressa®), lapatinib (Tykerb®) and those compounds that are generically and specifically disclosed in WO 97/02266, EP 0 564 409, WO 99/03854, EP 0 520 722, EP 0 566 226, EP 0 787 722, EP 0 837 063, U.S. Pat. No. 5,747,498, WO 98/10767, WO 97/30034, WO 97/49688, WO 97/38983 and WO 96/33980; farnesyltransferase inhibitors which include without limitation ahydroxyfarnesylphosphonic acid, butanoic acid, 2-[(2S)-2-[[(2S,3S)-2-[[(2R)-2-amino-3-mercaptopropyl]amino]-3-methylpent-yl]oxy]-1-oxo-3-phenylpropyl]amino]-4-(methylsulfonyl)-1-methylethylester (2S)-(9C1), tipifarnib (Zarnestra®), and manumycin A; Flk-1 kinase inhibitors which include without limitation 2-propenamide, 2-cyano-3-[4-hydroxy-3,5-bis(1-methylethyl)phenyl]-N-(3-phenylpropyl)-(2E-)-(9C1); glycogen synthase kinase-3 (GSK3) inhibitors which include without limitation indirubin-3′-monooxime; histone deacetylase (HDAC) inhibitors which include without limitation suberoylanilide hydroxamic acid (SAHA), [4-(2-amino-phenylcarbamoyl)-benzyl]carbamic acid pyridine-3-ylmethylester and its derivatives, butyric acid, pyroxamide, trichostatin A, oxamflatin, apicidin, depsipeptide, depudecin, trapoxin, vorinostat (Zolinza®), and compounds disclosed in WO 02/22577; I-kappa B-alpha kinase inhibitors (IKK) which include without limitation 2-propenenitrile, 3-[(4-methylphenyl)sulfonyl]-(2E)-(9C1); imidazotetrazinones which include without limitation temozolomide (Methazolastone®, Temodar® and its derivatives (e.g., as disclosed generically and specifically in U.S. Pat. No. 5,260,291) and Mitozolomide; insulin tyrosine kinase inhibitors which include without limitation hydroxyl-2-naphthalenylmethylphosphonic acid; c-Jun-N-terminal kinase (INK) inhibitors which include without limitation pyrazoleanthrone and epigallocatechin gallate; mitogen-activated protein kinase (MAP) inhibitors which include without limitation benzenesulfonamide, N-[2-[[[3-(4-chlorophenyl)-2-propenyl]methyl]amino]methyl]phenyl]-N-(2-hydroxyethyl)-4-methoxy-(9C1); MDM2 inhibitors which include without limitation trans-4-iodo, 4′-boranyl-chalcone; MEK inhibitors which include without limitation butanedinitrile, bis[amino[2-aminophenyl)thio]methylene]-(9C1); MMP inhibitors which include without limitation Actinonin, epigallocatechin gallate, collagen peptidomimetic and non-peptidomimetic inhibitors, tetracycline derivatives marimastat (Marimastat®), prinomastat, incyclinide (Metastat®), shark cartilage extract AE-941 (Neovastat®), Tanomastat, TAA211, MMI270B or AAJ996; mTor inhibitors which include without limitation rapamycin (Rapamune®), and analogs and derivatives thereof, AP23573 (also known as ridaforolimus, deforolimus, or MK-8669), CCI-779 (also known as temsirolimus) (Torisel®) and SDZ-RAD; NGFR tyrosine kinase inhibitors which include without limitation tyrphostin AG 879; p38 MAP kinase inhibitors which include without limitation Phenol, 4-[4-(4-fluorophenyl)-5-(4-pyridinyl)-1H-imidazol-2-yl]-(9C1), and benzamide, 3-(dimethylamino)-N-[3-[(4-hydroxylbenzoyl)amino]-4-methylphenyl]-(9C1); p56 tyrosine kinase inhibitors which include without limitation damnacanthal and tyrphostin 46; PDGF pathway inhibitors which include without limitation tyrphostin AG 1296, tyrphostin 9, 1,3-butadiene-1,1,3-tricarbonitrile, 2-amino-4-(1H-indol-5-yl)-(9C1), imatinib (Gleevec®) and gefitinib (Iressa®) and those compounds generically and specifically disclosed in European Patent No.: 0 564 409 and PCT Publication No.: WO 99/03854; phosphatidylinositol 3-kinase inhibitors which include without limitation wortmannin, and quercetin dihydrate; phosphatase inhibitors which include without limitation cantharidic acid, cantharidin, and L-leucinamide; protein phosphatase inhibitors which include without limitation cantharidic acid, cantharidin, L-P-bromotetramisole oxalate, 2(5H)-furanone, 4-hydroxy-5-(hydroxymethyl)-3-(1-oxohexadecyl)-(5R)-(9C1) and benzylphosphonic acid; PKC inhibitors which include without limitation 1-H-pyrollo-2,5-dione, 3-[1-3-(dimethylamino)propyl]-1H-indol-3-yl]-4-(1H-indol-3-yl)-(9C1), Bisindolylmaleimide IX, Sphinogosine, staurosporine, and Hypericin; PKC delta kinase inhibitors which include without limitation rottlerin; polyamine synthesis inhibitors which include without limitation DMFO; PTP1B inhibitors which include without limitation L-leucinamide; protein tyrosine kinase inhibitors which include, without limitation tyrphostin Ag 216, tyrphostin Ag 1288, tyrphostin Ag 1295, geldanamycin, genistein and 7H-pyrrolo[2,3-d]pyrimidine derivatives as generically and specifically described in PCT Publication No.: WO 03/013541 and U.S. Publication No.: 2008/0139587; SRC family tyrosine kinase inhibitors which include without limitation PP1 and PP2; Syk tyrosine kinase inhibitors which include without limitation piceatannol; Janus (JAK-2 and/or JAK-3) tyrosine kinase inhibitors which include without limitation tyrphostin AG 490 and 2-naphthyl vinyl ketone; retinoids which include without limitation isotretinoin (Accutane®, Amnesteem®, Cistane®, Claravis®, Sotret®) and tretinoin (Aberel®, Aknoten®, Avita®, Renova®, Retin-A®, Retin-A MICRO®, Vesanoid®); RNA polymerase H elongation inhibitors which include without limitation 5,6-dichloro-1-beta-D-ribofuranosylbenzimidazole; serine/Threonine kinase inhibitors which include without limitation 2-aminopurine; sterol biosynthesis inhibitors which include without limitation squalene epoxidase and CYP2D6; VEGF pathway inhibitors, which include without limitation anti-VEGF antibodies, e.g., bevacizumab, and small molecules, e.g., sunitinib (Sutent®), sorafinib (Nexavar®), ZD6474 (also known as vandetanib) (Zactima™), SU6668, CP-547632 and AZD2171 (also known as cediranib) (Recentin™).

### EXAMPLES

Some aspects of the embodiments discussed above are disclosed in further detail in the following examples, which are not in any way intended to limit the scope of the present disclosure.

### Example 1

### Heat-Inducible Macrophages

Summary

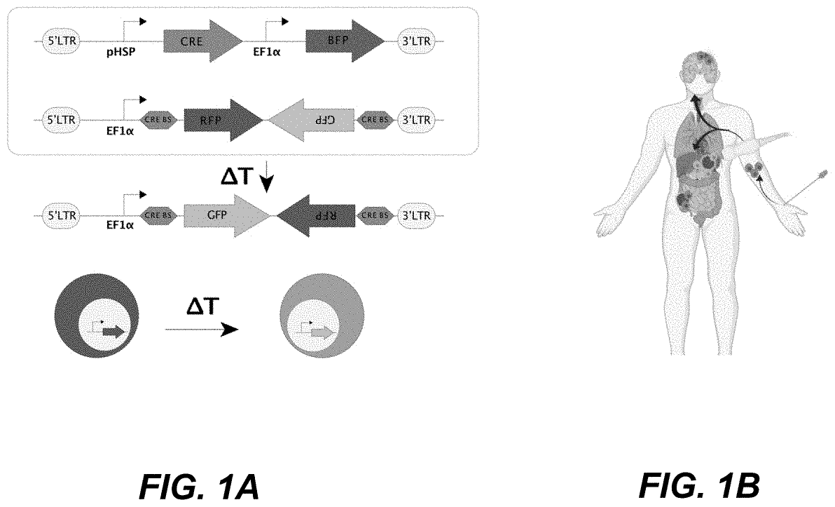

Provided herein include FUS-controlled macrophages in which a brief elevation of temperature results in stable gene activation. This platform can comprise two components (FIG. 1A) which are coupled to enable optimal circuit functionality. The first is the endogenous heat shock pathway (HSP) machinery of mammalian cells. In response to extrinsic or intrinsic stresses upon the cell that result in the accumulation of unfolded protein in the cytoplasm, HSP70/90 chaperones relieve HSF1, a transcription factor, from sequestration. HSF1 traffics to the nucleus, where it trimerizes and activates transcription of homeostatic genes from HSP promoters. These promoters can be harnessed to a transgene to enable thermally regulated gene expression.

In some embodiments, the second component of the circuit couples HSP promoter activation to Cre recombinase expression. This recombinase can catalyze inversion or deletion events, depending on the orientation of the loxP DNA-binding sites. In some embodiments of the circuit, the loxP sites are oriented such that inversion is catalyzed in the presence of the Cre recombinase. Upon activation of HSP promoters, Cre recombinase can be expressed and catalyzes the inversion of DNA flanked by loxP sites. This can enable thermal control over a state switch, through control of open-reading frame (ORF) orientation relative to an upstream promoter. This ORF can encode protein therapeutics (e.g. payloads) such as nanobodies, cytokines, chemokines, enzymes or bispecific T-cell engagers, or genome editing or RNA-acting components that alter the expression of endogenous genes to alter the cell state. Additionally, this state switch can be used to halt transcription of a specific ORF, once it is inverted. In some embodiments, this ORF encodes a kill-switch, which, once switched off, enables only cells which have received the thermal signal to survive. Multiple ORFs can be combined through 2A, IRES or similar mechanisms as provided herein.

Alternatively, in some embodiments, feedforward and/or positive feedback architectures are employed to drive expression of the ORF output(s) of interest in macrophages in response to a thermal signal. These circuits can make use of a transactivator whose expression is driven by an HSP promoter. Once the thermal stimulus has been applied, the transactivator can be transcribed and can activate transcription from a matching promoter upstream of the ORF. This feedforward step can enable an amplification of the signal, leading to persistence over days. In addition, the matching promoter can be placed upstream of the transactivator (e.g., in series with the HSP), such that the transactivator continues to drive its own expression, as well as that of the output ORF, in the absence of further heating.

Once macrophages have been engineered with the described circuit, these macrophages can be delivered systemically (FIG. 1B). These macrophages can accumulate in tumors, after which the human operator can apply heat in the form of FUS or other thermal energy-depositing methods (e.g. infrared light, warm liquid solution injections) to activate the circuit. These macrophages, having made use of physiological signals to home to tumors and an exogenous thermal signal to activate recombinase expression, can release immunomodulators or other protein therapeutics to enable high local dosing with minimal off-target expression.

Constructing and Evaluating a Thermal State Switch in Macrophages

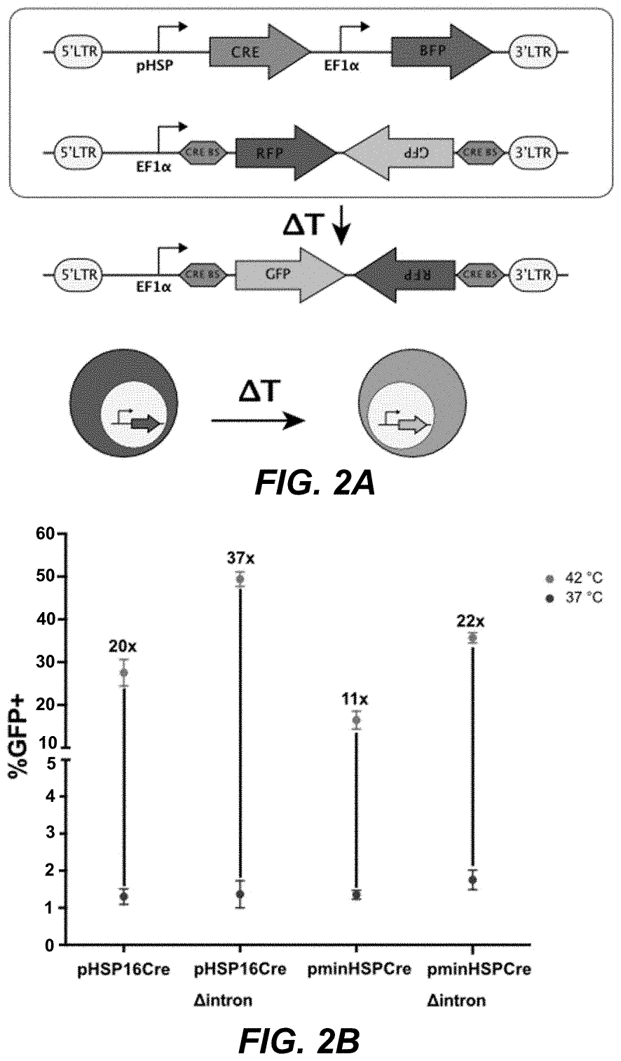

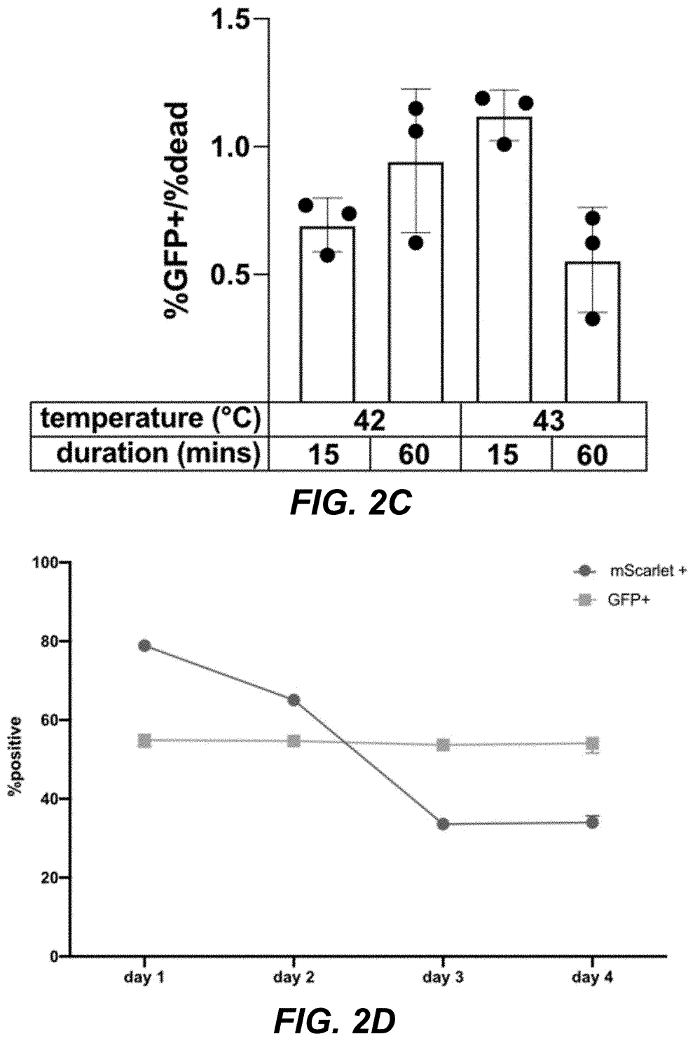

To develop a temperature-actuated genetic circuit in macrophages two HSP promoters (HSP16 and minHSP) were employed. These two candidate HSP promoters were placed upstream of the Cre recombinase gene, enabling transcription of the recombinase to be coupled to temperature elevation. This circuit was constructed within a lentiviral vector as shown in FIG. 2A. The Cre recombinase gene employed contained a synthetic intron to prevent expression in the bacterial cloning host, and it was hypothesized that the inclusion of the intron would increase the size of the lentiviral vector, thus reducing the efficiency of transduction. This led to the creation of four candidate HSP-Cre lentiviral vectors, using either HSP16 or minHSP promoters with the full length Cre recombinase gene or the intronic deletion (Δintron).

In a separate lentiviral vector, an inverted recombinase reporter was constructed in which a constitutive EF1α promoter drives production of a mScarlet reporter placed immediately upstream of an inverted GFP reporter. The mScarlet and GFP reporters are flanked by loxP sites. When Cre recombinase is introduced into the system, the target DNA between the loxP sites is inverted, leading to expression of GFP and a loss of expression of mScarlet.

The four candidate HSP-Cre lentiviral vectors were used to coinfect RAW264.7 macrophages with the inverted recombinase reporter to generate stable cell lines. After verification of successful infection with the BFP infection marker as well as mScarlet expression, the performance of each candidate HSP-Cre architecture was evaluated. Cells were heated at 37° C. or 42° C. for 1 hour in a thermocycler, after which they were analyzed 24 hours later via flow cytometry. To identify the number of cells that had switched on that were capable of switching on, the flow cytometry data was first gated for BFP+ cells, as these were cells that had received the HSP-Cre lentiviral vector. Next, the percentage of these cells that were GFP+ from both treatments (37° C. or 42° C.) was examined (FIG. 2B). Thresholds for mScarlet+, GFP+, and BFP+ cells were determined using wild-type RAW264.7 cells.

Remarkably, these circuits displayed very low activation at 37° C., with high fold-induction following temperature elevation at 42° C. The optimal circuit was found to use HSP16 with the intron deletion in the downstream Cre recombinase. This circuit was used for all subsequent experiments. In order to make use of these circuits therapeutically, the heating parameters are optimized to reduce tissue damage and ensure efficient switching of the genetic circuits. In addition, clinical FUS makes use of MRI thermometry to ensure that the temperature of the target tissue is not elevated to the point of injury. Due to the high-cost of MRI, and the patient discomfort that accompanies long MRI times, the amount of time spent performing FUS should be minimized. Keeping these clinical considerations in mind, four heating conditions were assayed for their impact on circuit activation in our engineered reporter cell line. These heating conditions were also assayed for their effect on cell viability in wild-type RAW264.7 cells. Surprisingly, treatment of cells at 43° C. for 15 minutes was sufficient to activate a large percentage of BFP+ cells and had a comparable impact on viability to longer heating durations at 42° C. (FIG. 2C). This reduction in heating time allows the alleviation of clinical concerns over extended MRI usage for FUS targeting.

In some embodiments, in order to use temperature-controlled state switches to alter the immunosuppressive nature of the tumor in a spatially restricted manner, these state switches must be stable following thermal stimulus application. To test the stability of the state switches in vitro, the engineered reporter line was treated with temperature elevation to 43° C. for 15 minutes and then analyzed the numbers of mScarlet+ cells and GFP+ cells over time. The levels of GFP+ cells remained constant over 4 days following temperature elevation, indicating that the circuit is stable over time. The levels of mScarlet+ cells decreases over time, due to dilution of the fluorescent protein over time, indicating that recombination had switched off expression of mScarlet (FIG. 2D). The temperature-sensitive state switch designed here can be activated over a very short duration of heating, and the resulting state is highly stable over time. This state switch provided the optimal platform for control of immunotherapeutic agents.

Thermal State Switches can Control Release of Immunotherapeutics

To demonstrate that this circuit can control the release of immunotherapeutic agents, key immunomodulators with demonstrated clinical efficacy were identified. Blinatumomab is a bispecific T-cell engager (BiTE) which binds the CD3 antigen on T cells and the CD19 antigen on target cancer cells. This therapeutic has been used in the treatment of CD19+ hematological malignancies. While blinatumomab has demonstrated clinical efficacy, its usage is associated with numerous neurological side effects, which some studies have suggested occur in 98% of patients. The direct mechanism for neurological side effects is still unknown, but recent work suggests that low levels of CD19 expression in mural cells in the brain result in targeting of blinatumomab and other CD19-targeted therapeutics to the brain, resulting in neurological side effects. These side effects can thus be characterized as on-target, off-tumour; molecular specificity is insufficient to restrict immunomodulation to cancer cells. As such, this was identified as an excellent candidate for release with the temperature-controlled state switch.

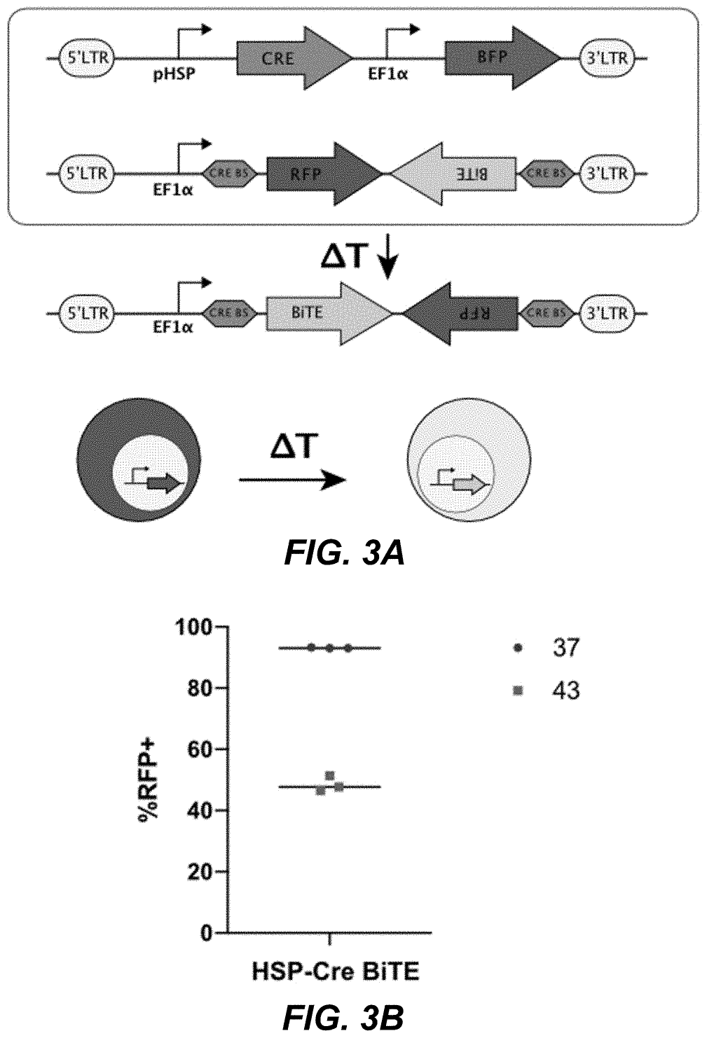

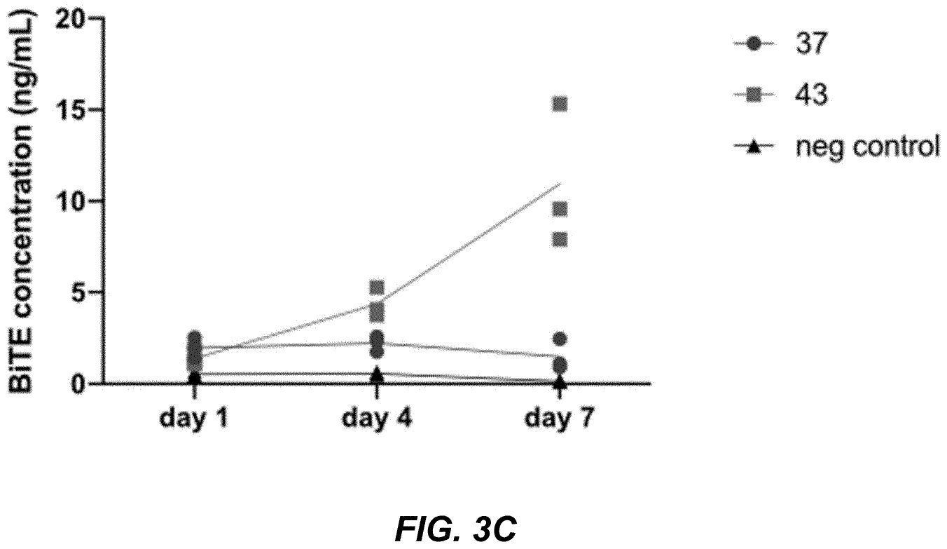

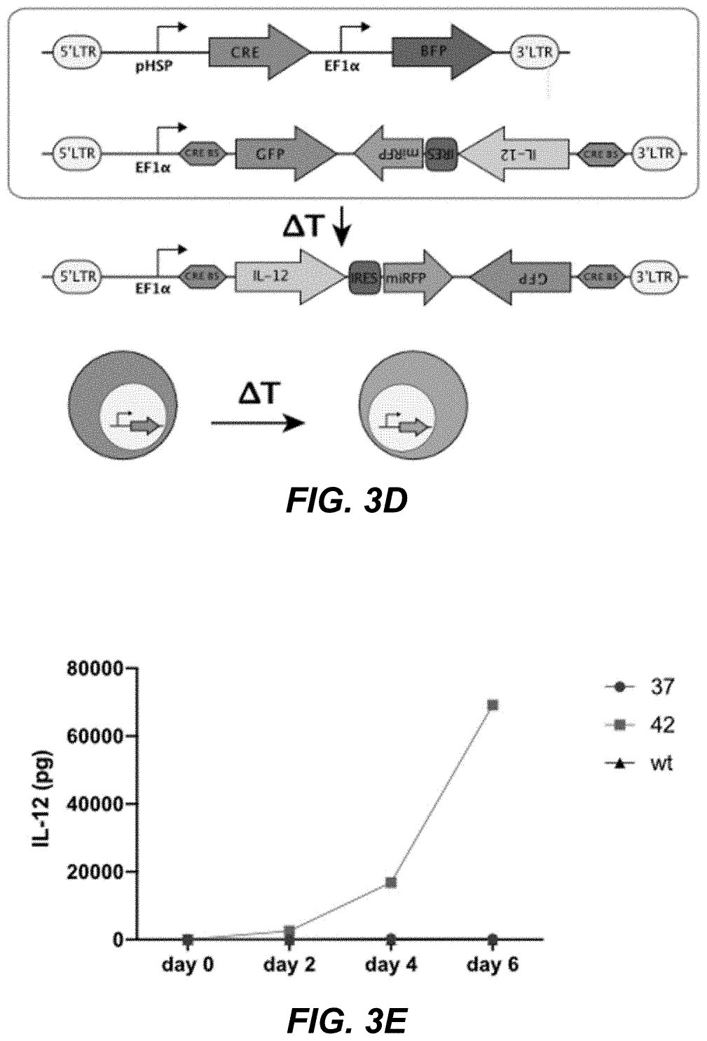

To control these therapeutics with a disclosed temperature-sensitive state switch, the design of the inverted reporter construct was modified to replace GFP with a gene encoding blinatumomab, denoted as “BiTE” (FIG. 3A). The lentiviral vectors were then separately used to coinfect RAW264.7 cells with the pHSP16-Cre lentiviral vector and were then sorted using flow cytometry for high expression of BFP and mScarlet. This was performed to select non-switched cells (which would still express mScarlet) with the highest probability of switching following temperature elevation.

Once stable RAW264.7 macrophages had been generated and sorted appropriately, it was verified that the state switch still functioned in a similar manner to the inverted mScarlet-GFP reporter. Temperature elevation of the therapeutic circuit was designed to switch off mScarlet expression, and it was hypothesized that this switching off should follow similar dilution kinetics as the inverted mScarlet-GFP reporter. To test this, cells were treated with elevated temperature 43° C. for 15 minutes and compared to control cells maintained at 37° C. These cells were analyzed using flow cytometry four days after temperature elevation (FIG. 3B). A marked decrease in the percentage of cells expressing mScarlet was observed as expected, indicating that the recombinase action was preserved in the therapeutic circuit.

After validating the genetic architecture of the therapeutic state switch, the release of blinatumomab from the engineered macrophages was quantified. Since blinatumomab was tagged with an N-terminal His-tag, competitive anti-His Tag ELISA was used to quantify release kinetics after temperature elevation. To test this, 0.5×10⁶ macrophages were seeded in 24-well plates following temperature elevation to 43° C. for 15 minutes. Engineered macrophages harboring the same genetic circuit maintained at 37° C. were used as controls. At multiple timepoints following temperature elevation, supernatant was removed from the cell culture and analyzed using competitive anti-His Tag ELISA (FIG. 3C). As expected, the baseline BiTE secretion remained low with cells maintained at 37° C. for the duration of the experiment, while the concentration rose almost 5-fold over 7 days with cells treated with temperature elevation. This demonstrated the unique ability of the disclosed temperature-sensitive state switches to control therapeutic release from engineered macrophages with very low baseline release, using a clinically applicable temperature elevation regimen.

FIG. 3D depicts a non-limiting exemplary illustration of illustration of a genetic circuit controlling release of mouse IL-12 (m-IL-12). This circuit uses a modified design in which an additional fluorescent protein, miRFP, is expressed downstream of the therapeutic. This enables the user to assess inversion of the circuit following heating by flow cytometry. FIG. 3E depicts data related to a IL-12 ELISA (R&D Biosystems) directly detecting mIL12 on supernatant of IL-12 secreting RAW264.7 cells after heating at 42 C for 1 hour.

Thermal State Switch Activation with Focused Ultrasound

After verifying migration of engineered macrophages into tumours of interest, the activation of cells within the tumour using focused ultrasound was attempted. In order to test this, 60×10⁶ RAW264.7 cells harboring the inverted RFP-GFP reporter were prepared and injected systemically over 3 days into NSG mice harboring subcutaneous Raji tumors after the tumours reached ˜100 mm³.

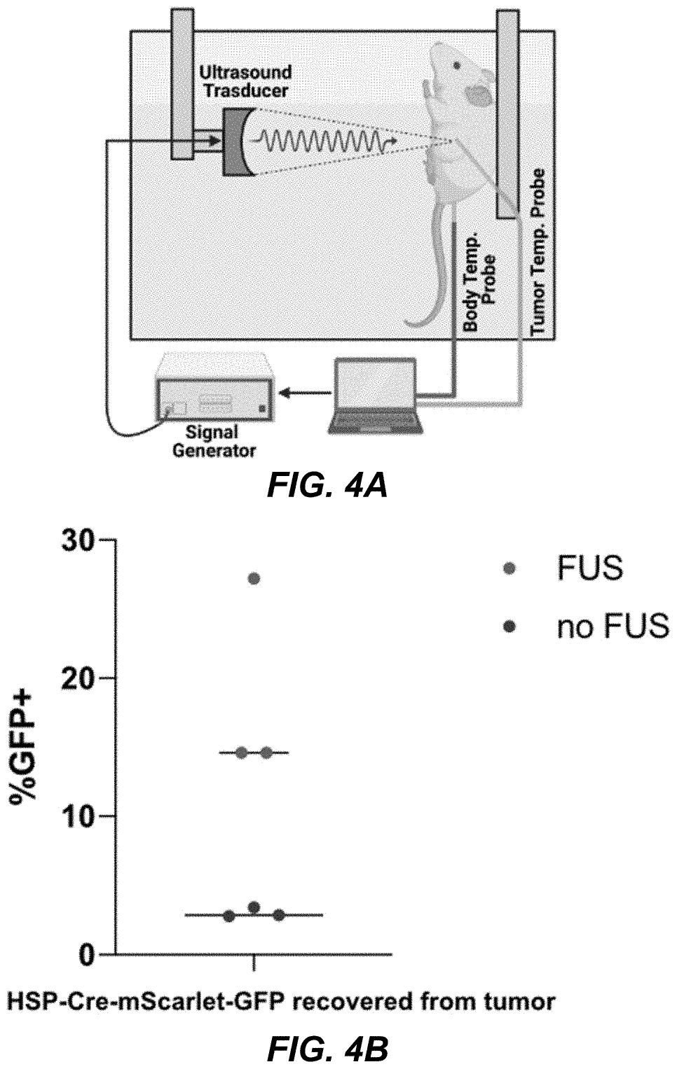

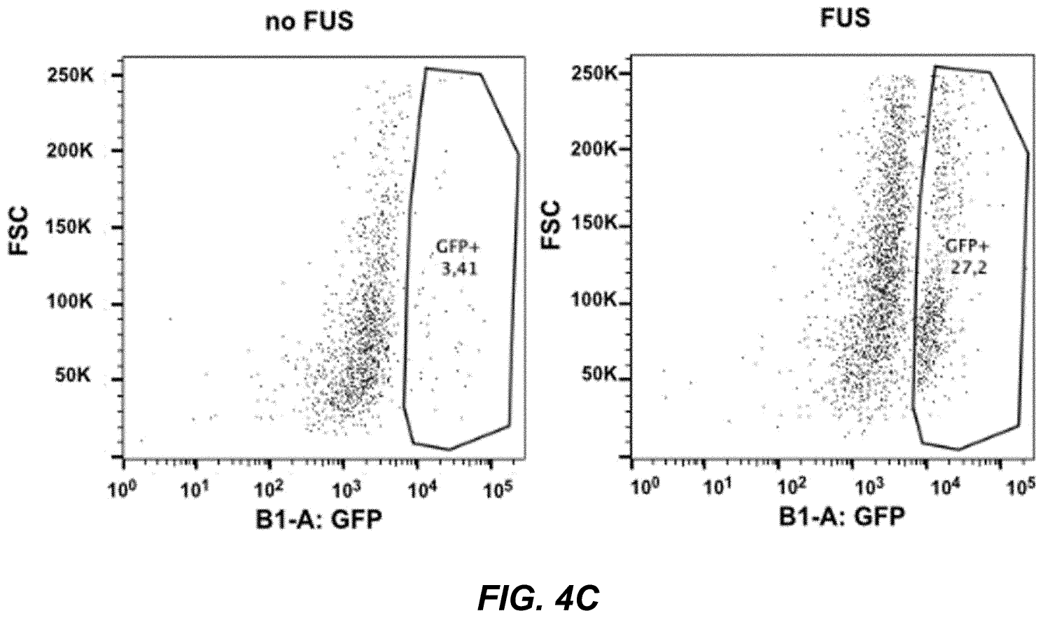

One day after the last injection, focused ultrasound was performed on the tumours, using a setup shown in FIG. 4A as described previously (Abedi, M. H., Lee, J., Piraner, D. I., & Shapiro, M. G. (2020). Thermal Control of Engineered T-cells. ACS synthetic biology, 9(8), 1941-1950). The temperature of the tumour was monitored using fiber optic thermometry, and this temperature monitoring was used for feedback control of focused ultrasound intensity, to ensure that the tumor was maintained at the desired temperature. Tumours were heated to 43° C. for 15 minutes while mice were anesthetized with isoflurane and submerged in a water tank. One day after heating, mice were euthanized and tumors were harvested and homogenized for analysis with flow cytometry.

In order to determine the extent of switching of cells within the tumour, mScarlet+ cells were first gated upon as described previously. Since the analysis of tumors was performed only a day after focused ultrasound application, it was assumed that mScarlet would not have been diluted significantly, enabling us to use this gate to identify the engineered macrophages. After selecting this population, GFP+ cells were identified as shown below (FIGS. 4B-4C). The percentage of GFP+ cells was consistent with in vitro analyses of the inverted RFP-GFP reporter circuit, with the percentage of activated cells being comparable to that observed in vitro (FIG. 2B).

Generation and Testing IC-21 Heat-Inducible Macrophages

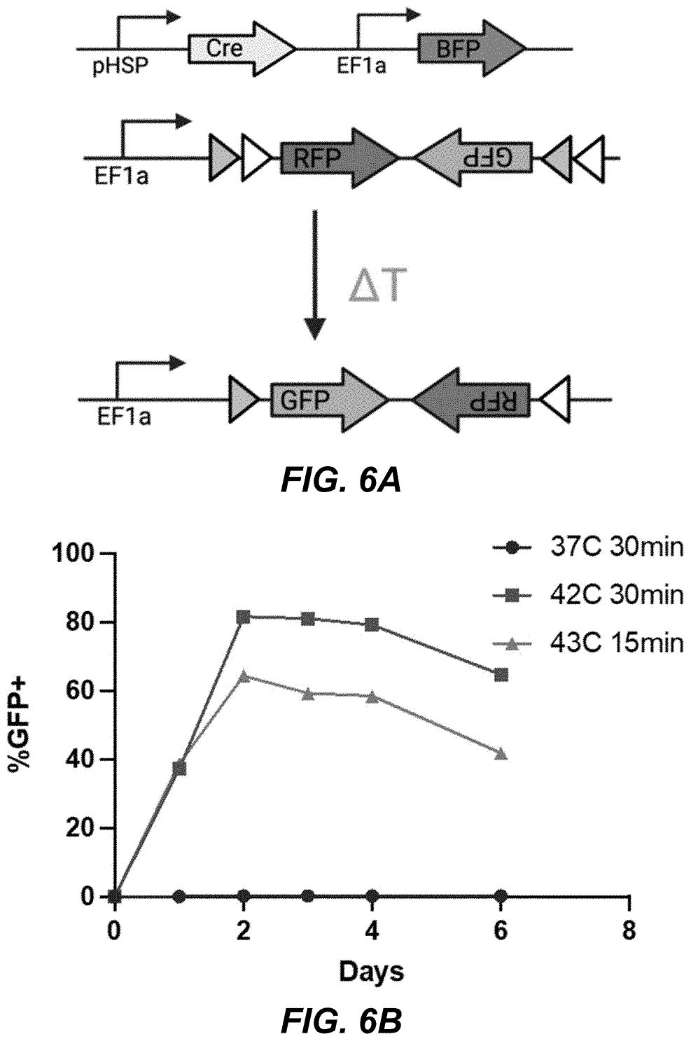

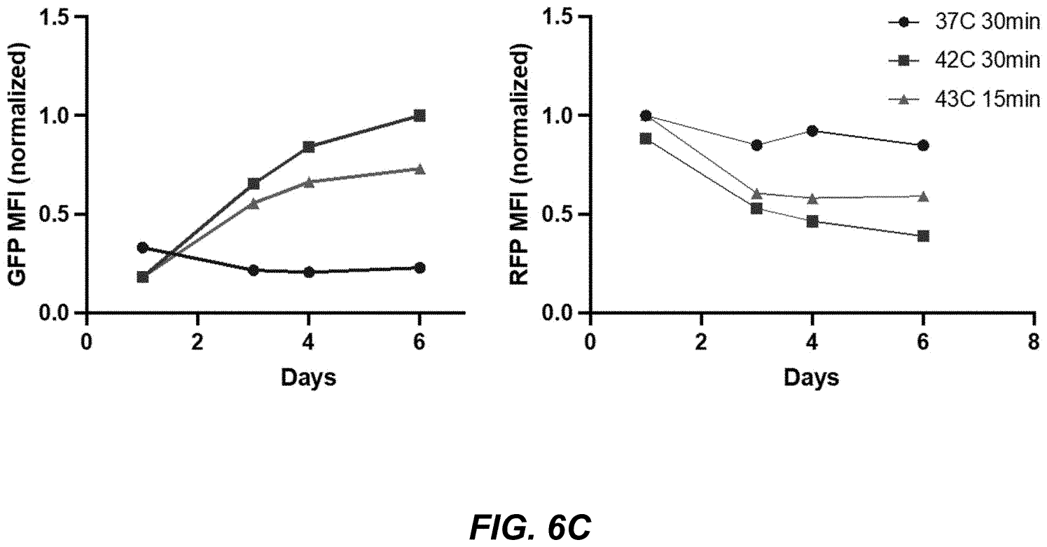

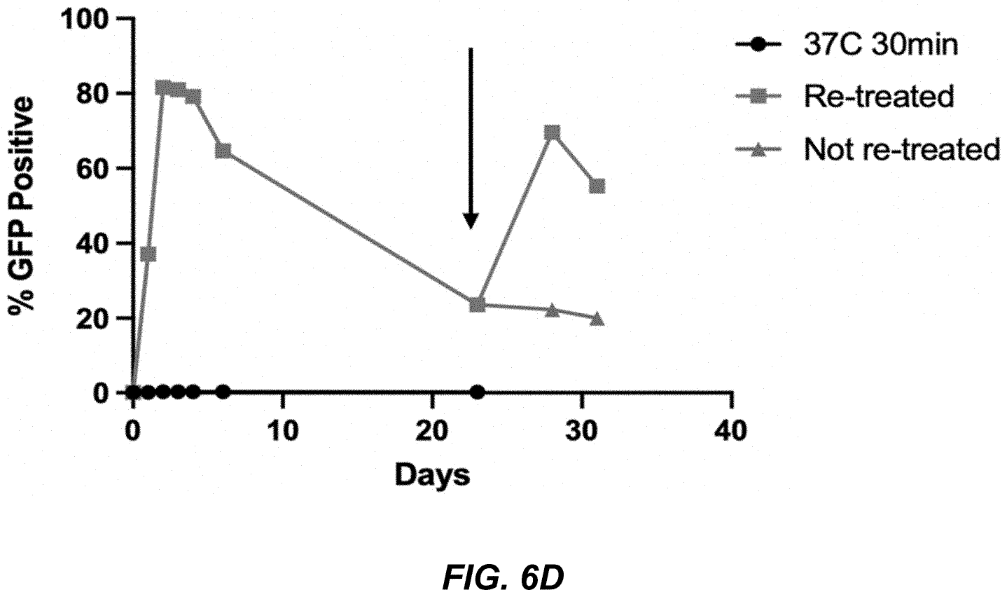

For further proof of principle, the constructs provided herein were tested in an additional macrophage cell line, IC-21. FIGS. 6A-6D depict non-limiting exemplary schematics and data related to IC-21 heat-inducible macrophages. FIG. 6A depicts a non-limiting exemplary schematic showing a temperature-sensitive state switch genetic circuit design. This circuit was lentivirally transduced into the C57BL/6 mouse peritoneal macrophage cell line, IC-21. FIG. 6B depicts data related to the percent of activated (GFP+) IC-21 cells measured using flow cytometry over 6 days after incubation at 37° C., 42° C., or 43° C. FIG. 6C depicts data related to mean fluorescence intensity of GFP (indicating active state) and RFP (indicating inactive state) of IC-21 cells over 6 days after incubation at 37° C., 42° C., or 43° C. FIG. 6D depicts data related to percent of activated (GFP+) IC-21 cells after incubation at 37° C. or 42° C. for 30 minutes. The cells were treated again 23 days after the initial incubation (indicated by the black arrow). These results provided additional proof of principle for the heat-inducible macrophage platforms provided herein.

Methods

Temperature Elevation Assay

Temperature elevation of macrophages was performed in a Bio-Rad C1000 thermocycler. Macrophages were mixed well before transferring 50 μL into a sterile PCR tube. The temperature and duration of stimulation was varied based on the experimental procedure. Upon completion of thermal stimulation, cells were moved back into a mammalian incubator and supplemented with fresh media. Cells were typically incubated for 24 hours unless stated otherwise before assaying with a flow cytometer (MACSQuant VYB). Dead cells were typically excluded via FSC/SSC gating for routine assays. In FIGS. 1A-1B, a LIVE/DEAD viability/cytotoxicity kit (Thermo Fisher) was used for a more accurate quantification of cell state. Live cells were further gated via a fluorescent protein to isolate virally infected cells for further analysis. Thresholds for BFP, GFP+ and mScarlet+ were determined using wild-type RAW264.7 cells. Blinatumomab expression was measured using a competitive Anti His Tag ELISA (GenScript).

Preparation of Cell Lines for In Vitro and In Vivo Experiments

RAW264.7 (TIB-71) were obtained from ATCC and cultured in DMEM media (Thermo Fisher Scientific) with 1× Penicillin/Streptomycin (Corning). All engineered cell lines were constructed via lentiviral infection. Lentivirus was prepared using a third-generation viral vector and helper plasmids (gifts of D. Baltimore). Virus was packaged in HEK293T cells grown in 10 cm dishes. After 3 days of transfection viral particles were concentrated via ultracentrifugation. Infection was performed using addition of Polybrene (Millipore Sigma) at 5 μg/mL. Experiments were performed at least two weeks after infection.

Feedback-Controlled Focused Ultrasound

A closed loop thermal control setup was developed to maintain a specified predetermined temperature within the tumor of a mouse by modulating the intensity of the FUS. This setup includes a water bath filled with pure distilled water that was being actively cleaned and degassed with an AQUAS-10 water conditioner (ONDA) and maintained at 33° C. with a sous vide immersion cooker (InstantPot Accu Slim). A tumor-bearing mouse that has been anesthetized as described above was fastened nose up vertically to an acrylic arm that was connected to a manual 3D positioning system (Thorlabs) to enable 3D motion of the mouse within the water bath. A Velmex BiSlide motorized positioning system was used to submerge and position the 0.67 MHz FUS transducer (Precision Acoustics PA717) such that the focal point of the transducer lies inside the tumor of the mouse. A signal generator (B&K #4054B) generates the thermal ultrasound signal which was then amplified (AR #100A250B) and sent to drive the ultrasound transducer. The water in this chamber acts as the coupling medium to transfer the ultrasound wave from the transducer to the tumor. A thin fiber optic temperature probe (Neoptix) was temporarily implanted into the tumors to measure the internal tumor temperature during a heating session. This temperature readout was also used to align the focus of the transducer with the tumor by emitting a constant test thermal ultrasound signal. Once the system was aligned, a Matlab closed loop thermal control script was run that regulates the signal generator output. Feedback for the controller was provided by the temperature measurements acquired with a sampling rate of 4 Hz. The actuator for the controller was the voltage amplitude of the continuous sinusoidal signal at 0.67 MHz used to drive the FUS transducer, where the voltage was adjusted also at 4 Hz. The system uses a PID controller with anti-windup control that modifies the amplitude of the thermal ultrasound waveform to achieve a desired temperature in the targeted tissues. The Kp, Ki, Kd, and Kt parameters for the PID and anti-windup were tuned using Ziegler-Nichols method, and in some cases adjusted further through trial-and-error tuning to achieve effective thermal control.

Tumor Analysis

Tumors were collected and homogenized in ten milliliters of DMEM containing 2 mg/ml collagenase and 0.1 mg/ml DNAse for one hour at 37° C. 450 μL of the homogenized tumor was used for flow cytometry. To estimate the total cell counts within the tumour, the cell count obtained from flow cytometry of the sample was multiplied by the total volume of the homogenized tumour.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A population of heat-inducible macrophages, one or more of said heat-inducible macrophages comprising:
a first inducible promoter operably linked to a first polynucleotide comprising a recombinase gene,
wherein the first inducible promoter is capable of inducing transcription of the recombinase gene to generate a recombinase transcript upon thermal stimulation,
and wherein the recombinase transcript is capable of being translated to generate a recombinase; and
a second promoter and a second polynucleotide comprising a payload gene,
wherein, in the absence of a recombination event, the second promoter and the second polynucleotide are not operably linked,
wherein the recombinase is capable of catalyzing the recombination event, and
wherein the second promoter and the second polynucleotide are operably linked after the recombination event such that the second promoter is capable of inducing transcription of the payload gene to generate a payload transcript.

2. The population of claim 1, wherein the recombination event comprises removal of a sequence flanked by recombinase target sites or an inversion of a sequence flanked by recombinase target sites.

3. The population of claim 1, wherein the second polynucleotide is flanked by recombinase target sites.

4. The population of claim 1, wherein, prior to the recombination event, the sequence of the payload gene is inverted relative to the promoter.

5. The population of claim 1, comprising at least one stop cassette situated between the second promoter and the payload gene, wherein the stop cassette comprises one or more stop sequences, and wherein the one or more stop cassettes are flanked by recombinase target sites, wherein the at least one stop cassette is configured to prevent transcription of the payload gene and/or translation of the payload transcript, and wherein the one or more stop sequences comprise a polyadenylation signal, a stop codon, a frame-shifting mutation, or any combination thereof.

6. The population of claim 1, wherein the second promoter selected from the group comprising a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CBH promoter, or any combination thereof.

7. The population of claim 1, wherein the recombinase is Cre, Dre, Flp, KD, B2, B3, λ, HK022, HP1, γ6, ParA, Tn3, Gin, ΦC31, Bxb1, R4, derivatives thereof, or any combination thereof.

8. The population of claim 1, comprising:
a third polynucleotide comprising a default gene,
wherein, in the absence of a recombination event, the second promoter and the third polynucleotide are operably linked such that the second promoter is capable of inducing transcription of the default gene to generate a default transcript,
wherein the default transcript is capable of being translated to generate a default protein, and
wherein the second promoter and the third polynucleotide are not operably linked after the recombination event such that the second promoter is no longer capable of inducing transcription of the default gene to generate a default transcript.

9. The population of claim 8, wherein the default gene is a pro-death gene encoding a pro-death protein, wherein the pro-death protein is capable of halting cell growth and/or inducing cell death, and wherein the pro-death protein selected from the group comprising cytosine deaminase, iCasp9, thymidine kinase, Bax, Bid, Bad, Bak, BCL2L11, p53, PUMA, Diablo/SMAC, S-TRAIL, Cas9, Cas9n, hSpCas9, hSpCas9n, HSVtk, cholera toxin, diphtheria toxin, alpha toxin, anthrax toxin, exotoxin, pertussis toxin, Shiga toxin, shiga-like toxin Fas, TNF, caspase 2, caspase 3, caspase 6, caspase 7, caspase 8, caspase 9, caspase 10, caspase 11, caspase 12, purine nucleoside phosphorylase, or any combination thereof.

10. The population of claim 8, wherein the default gene is a pro-death gene encoding a pro-death protein, wherein the pro-death protein is capable of halting cell growth and/or inducing cell death in the presence of a pro-death agent, and wherein:
the pro-death protein comprises iCasp9 and the pro-death agent comprises AP20187;
the pro-death protein comprises Caspase-9 and the pro-death agent comprises AP1903;
the pro-death protein comprises HSV thymidine kinase (TK) and the pro-death agent comprises Ganciclovir (GCV), Ganciclovir elaidic acid ester, Penciclovir (PCV), Acyclovir (ACV), Valacyclovir (VCV), (E)-5-(2-bromovinyl)-2′-deoxyuridine (BVDU), Zidovuline (AZT), and/or 2′-exo-methanocarbathymidine (MCT);
the pro-death protein comprises Cytosine Deaminase (CD) and the pro-death agent comprises 5-fluorocytosine (5-FC);
the pro-death protein comprises Purine nucleoside phosphorylase (PNP) and the pro-death agent comprises 6-methylpurine deoxyriboside (MEP) and/or fludarabine (FAMP);
the pro-death protein comprises a Cytochrome p450 enzyme (CYP) and the pro-death agent comprises Cyclophosphamide (CPA), Ifosfamide (IFO), and/or 4-ipomeanol (4-IM);
the pro-death protein comprises a Carboxypeptidase (CP) and the pro-death agent comprises 4-[(2-chloroethyl)(2-mesyloxyethyl)amino]benzoyl-L-glutamic acid (CMDA), Hydroxy-and amino-aniline mustards, Anthracycline glutamates, and/or Methotrexate α-peptides (MTX-Phe);
the pro-death protein comprises Carboxylesterase (CE) and the pro-death agent comprises Irinotecan (IRT), and/or Anthracycline acetals;
the pro-death protein comprises Nitroreductase (NTR) and the pro-death agent comprises dinitroaziridinylbenzamide CB1954, dinitrobenzamide mustard SN23862, 4-Nitrobenzyl carbamates, and/or Quinones;
the pro-death protein comprises Horse radish peroxidase (HRP) and the pro-death agent comprises Indole-3-acetic acid (IAA) and/or 5-Fluoroindole-3-acetic acid (FIAA);
the pro-death protein comprises Guanine Ribosyltransferase (XGRTP) and the pro-death agent comprises 6-Thioxanthine (6-TX); the pro-death protein comprises a glycosidase enzyme and the pro-death agent comprises HM1826 and/or Anthracycline acetals;
the pro-death protein comprises Methionine-α,γ-lyase (MET) and the pro-death agent comprises Selenomethionine (SeMET); and/or
the pro-death protein comprises thymidine phosphorylase (TP) and the pro-death agent comprises 5′-Deoxy-5-fluorouridine (5′-DFU).

11. The population of claim 1, wherein thermal stimulation comprises heating to an activating temperature, and wherein the activating temperature is about 37.5° C., about 38.0° C., about 38.5° C., about 39.0° C., about 39.5° C., about 40.0° C., about 40.5° C., about 41.0° C., about 41.5° C., about 42.0° C., about 42.5° C., about 43.0° C., about 43.5° C., about 44.0° C., about 44.5° C., about 45.0° C., about 45.5° C., or about 46.0° C.

12. The population of claim 1, wherein the steady-state levels of the payload transcript are at least 1.1 higher upon thermal stimulation.

13. The population of claim 1, wherein the first inducible promoter comprises or is derived from a mammalian heat shock promoter (HSP) or a C. elegans HSP.

14. The population of claim 1, wherein the first inducible promoter comprises a nucleotide sequence that is at least 80%, 85%, 90%, 95%, 98%, 99%, or 100% identical to any one of SEQ ID NOS: 1-14.

15. The population of claim 1, wherein the payload transcript is capable of being translated to generate a payload protein, and wherein a payload protein is or comprises:
a component of a synthetic protein circuit;
a therapeutic protein or a variant thereof;
a CRE recombinase, GCaMP, a cell therapy component, a knock-down gene therapy component, a cell-surface exposed epitope, or any combination thereof;
a bispecific T cell engager (BiTE) selected from the group comprising obinutuzumab, mosunetuzumab, selicrelumab, blinatumomab, ertumaxomab, maxomab, AMV564, AFM13, REGN-1979, GEN-3013, or pasotuxizumab;
a cytokine selected from the group consisting of interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, granulocyte macrophage colony stimulating factor (GM-CSF), M-CSF, SCF, TSLP, oncostatin M, leukemia-inhibitory factor (LIF), CNTF, Cardiotropin-1, NNT-1/BSF-3, growth hormone, Prolactin, Erythropoietin, Thrombopoietin, Leptin, G-CSF, or receptor or ligand thereof;
a member of the TGF-β/BMP family selected from the group consisting of TGF-β1, TGF-β2, TGF-β3, BMP-2, BMP-3a, BMP-3b, BMP-4, BMP-5, BMP-6, BMP-7, BMP-8a, BMP-8b, BMP-9, BMP-10, BMP-11, BMP-15, BMP-16, endometrial bleeding associated factor (EBAF), growth differentiation factor-1 (GDF-1), GDF-2, GDF-3, GDF-5, GDF-6, GDF-7, GDF-8, GDF-9, GDF-12, GDF-14, mullerian inhibiting substance (MIS), activin-1, activin-2, activin-3, activin-4, and activin-5;
a member of the TNF family of cytokines selected from the group consisting of TNF-alpha, TNF-beta, LT-beta, CD40 ligand, Fas ligand, CD 27 ligand, CD 30 ligand, and 4-1 BBL;
a member of the immunoglobulin superfamily of cytokines selected from the group consisting of B7.1 (CD80) and B7.2 (B70);
an interferon;
a chemokine selected from the group comprising CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13, or CXCL15;
an interleukin selected from the group comprising IL-10 IL-12, IL-1, IL-6, IL-7, IL-15, IL-2, IL-18 or IL-21;
an agonistic or antagonistic antibody or antigen-binding fragment thereof specific to a checkpoint inhibitor or checkpoint stimulator molecule;
a dosage indicator protein selected from the group comprising green fluorescent protein (GFP), enhanced green fluorescent protein (EGFP), yellow fluorescent protein (YFP), enhanced yellow fluorescent protein (EYFP), blue fluorescent protein (BFP), mScarlet, red fluorescent protein (RFP), TagRFP, Dronpa, Padron, mApple, mCherry, mruby3, rsCherry, rsCherryRev, derivatives thereof, or any combination thereof;
a nuclear localization signal (NLS) or a nuclear export signal (NES);
a constitutive signal peptide for protein degradation;
a secretion tag selected from the group comprising AbnA, AmyE, AprE, BglC, BglS, Bpr, Csn, Epr, Ggt, GlpQ, HtrA, LipA, LytD, MntA, Mpr, NprE, OppA, PbpA, PbpX, Pel, PelB, PenP, PhoA, PhoB, PhoD, PstS, TasA, Vpr, WapA, WprA, XynA, XynD, YbdN, Ybxl, YcdH, YclQ, YdhF, YdhT, YfkN, YflE, YfmC, Yfnl, YhcR, YlqB, YncM, YnfF, YoaW, YocH, YolA, YqiX, Yqxl, YrpD, YrpE, YuaB, Yurl, YvcE, YvgO, YvpA, YwaD, YweA, YwoF, YwtD, YwtF, YxaLk, YxiA, and YxkC;
a tumor necrosis factor (TNF) selected from the group comprising TNF-alpha, TNF-beta, TNF-gamma, CD252, CD154, CD178, CD70, CD153, or 4-1BBL; and/or
a chimeric antigen receptor (CAR) or T-cell receptor (TCR).

16. The population of claim 1, wherein the payload transcript is capable of being translated to generate a payload protein, and wherein a payload protein is capable of remodeling a tumor microenvironment.

17. The population of claim 1, wherein the heat-inducible macrophages do not comprise an exogenous receptor or targeting moiety configured to bind a component of a target site of a subject.

18. The population of claim 1, wherein the heat-inducible macrophages:
are derived from blood, cord blood, bone marrow, or iPSC;
are autologous macrophages and/or allogeneic macrophages; and/or
are Kupffer cells, stellate macrophages, M1 macrophages, M2 macrophages, tumor-associated macrophages (TAMs), or any combination thereof.

19. A method of treating a disease or disorder in a subject, the method comprising:
administering to the subject an effective amount of the population of heat-inducible macrophages of claim 1.

20. The method of claim 19, further comprising applying thermal energy to a target site of the subject sufficient to increase the local temperature of the target site to an activating temperature, thereby inducing the expression of the payload in heat-inducible macrophages at the target site.


SEQUENCE LISTING (14 sequences)

SEQ ID NO: 1 - DNA, 483 bp, synthetic construct
  source 1..483: mol_type = other DNA
aattagcttgaggatcctccacagccccggggagaccttgcctctaaagttgctgcttttgcagctctgccacaaccgcgcgtcctcagagccagccgggaggagctagaaccttccccgcgtttctttcagcagccctgagtcagaggcgggctggccttgcaagtagccgcccagccttcttcggtctcacggaccgatccgcccgaaccttctcccggggtcagcgccgcgctgcgccgcccggctgactcagcccgggcgggcgggcgggaggctctcgactgggcgggaaggtgcgggaaggttcgcggcggcggggtcggggaggtgcaaaaggatgaaaagcccgtggacggagctgagcagatccggccgggctggcggcagagaaaccgcagggagagcctcactgctgagcgcccctcgacgcgggcggcagcagcctccgtggcctccagcatccgacaagaagcttcagcc

SEQ ID NO: 2 - DNA, 538 bp, synthetic construct
  source 1..538: mol_type = other DNA
aattagcttgagcctctaaagttgctgcttttgcagcctctgccacaaccgcgcgtcctcagagccagcccggaggagctagaaccttccccgcatttctttcagcagcctgagtcagaggcgggctggcctggcgtagccgcccagcctcgcggctcatgccccgatctgcccgaaccttctcccggggtcagcgccgcgccgcgccacccggctgagtcagcccgggcgggcgagaggctctcaactgggcgggaaggtgcgggaaggtgcggaaaggttcgcgaaagttcgcggcggcgggggtcgggtgaggcgcaaaaggataaaaagccggtggaagcggagctgagcagatccgagccgggctggctgcagagaaaccgcagggagagcctcactgctgagcgcccctcgacggcggagcggcagcagcctccgtggcctccagcatccgacaagaagcttgaattcgagctcgccggggatcctctagtcagctgacgcgtgctagcgcggccgcaccactagtgccacc

SEQ ID NO: 3 - DNA, 526 bp, synthetic construct
  source 1..526: mol_type = other DNA
aattagcttgagcctctaaagttgctgcttttgcagcctctgccacaaccgcgcgtcctcagagccagcccggaggagctagaaccttccccgcatttctttcagcagcctgagtcagaggcgggctggcctggcgtagccgcccagcctcgcggctcatgccccgatctgcccgaaccttctcccggggtcagcgccgcgccgcgccacccggctgagtcagcccgggcgggcgagaggctctcaactgggcgggaaggtgcgggaaggtgcggaaaggttcgcgaaagttcgcggcggcgggggtcgggtgaggcgcaaaaggataaaaagccggtggaagcggagctgagcagatccgagccgggctggctgcagagaaaccgcagggagagcctcactgctgagcgcccctcgacggcggagcggcagcagcctccgtggcctccagcatccgacaagaagcttgaattcgagctcgccggggatcctctagtcagctgacgcgtgctagcgcggcgccacc

SEQ ID NO: 4 - DNA, 473 bp, synthetic construct
  source 1..473: mol_type = other DNA
aattagcttgagcctctaaagttgctgcttttgcagcctctgccacaaccgcgcgtcctcagagccagcccggaggagctagaaccttccccgcatttctttcagcagcctgagtcagaggcgggctggcctggcgtagccgcccagcctcgcggctcatgccccgatctgcccgaaccttctcccggggtcagcgccgcgccgcgccacccggctgagtcagcccgggcgggcgagaggctctcaactgggcgggaaggtgcgggaaggtgcggaaaggttcgcgaaagttcgcggcggcgggggtcgggtgaggcgcaaaaggataaaaagccggtggaagcggagctgagcagatccgagccgggctggctgcagagaaaccgcagggagagcctcactgctgagcgcccctcgacggcggagcggcagcagcctccgtggcctccagcatccgacaagaagcttcagcc

SEQ ID NO: 5 - DNA, 323 bp, synthetic construct
  source 1..323: mol_type = other DNA
aattagcttgaccccgatctgcccgaaccttctcccggggtcagcgccgcgccgcgccacccggctgagtcagcccgggcgggcgagaggctctcaactgggcgggaaggtgcgggaaggtgcggaaaggttcgcgaaagttcgcggcggcgggggtcgggtgaggcgcaaaaggataaaaagccggtggaagcggagctgagcagatccgagccgggctggctgcagagaaaccgcagggagagcctcactgctgagcgcccctcgacggcggagcggcagcagcctccgtggcctccagcatccgacaagaagcttcagcc

SEQ ID NO: 6 - DNA, 323 bp, synthetic construct
  source 1..323: mol_type = other DNA
aattagcttgaccccgatctgcccgaaccttctcccggggtcagcgccgcgccgcgccacccggctgcagcagcccgggcgggcgagaggctctcaactgggcgggaaggtgcgggaaggtgcggaaaggttcgcgaaagttcgcggcggcgggggtcgggtgaggcgcaaaaggataaaaagccggtggaagcggagctgagcagatccgagccgggctggctgcagagaaaccgcagggagagcctcactgctgagcgcccctcgacggcggagcggcagcagcctccgtggcctccagcatccgacaagaagcttcagcc

SEQ ID NO: 7 - DNA, 628 bp, synthetic construct
  source 1..628: mol_type = other DNA
aattagcttgaccccgatctgcccgaaccttctcccggggtcagcgccgcgccgcgccacccggctgcagcagcccgggcgggcgagaggctctcaactgggcgggaaggtgcgggaaggtgcggaaaggttcgcgaaagttcgcggccggactagagtggcgagatcccccgatctgcccgaaccttctcccggggtcagcgccgcgccgcgccacccggctgcagcagcccgggcgggcgagaggctctcaactgggcgggaaggtgcgggaaggtgcggaaaggttcgcgaaagttcgcggcaattagcttgaccccgatctgcccgaaccttctcccggggtcagcgccgcgccgcgccacccggctgcagcagcccgggcgggcgagaggctctcaactgggcgggaaggtgcgggaaggtgcggaaaggttcgcgaaagttcgcggcggcgggggtcgggtgaggcgcaaaaggataaaaagccggtggaagcggagctgagcagatccgagccgggctggctgcagagaaaccgcagggagagcctcactgctgagcgcccctcgacggcggagcggcagcagcctccgtggcctccagcatccgacaagaagcttcagcc

SEQ ID NO: 8 - DNA, 476 bp, synthetic construct
  source 1..476: mol_type = other DNA
aattagcttgagccgcccactcccccttcctctcagggtccctgtcccctccagtgaatcccagaagactctggagagttctgagcagggggcggcactctggcctctgattggtccaaggaaggctggggggcaggacgggaggcgaaaaccctggaatattcccgacctggcagcctcatcgagctcggtgattggctcagaagggaaaaggcgggtctccgtgacgacttataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaaccgcc

SEQ ID NO: 9 - DNA, 774 bp, synthetic construct
  source 1..774: mol_type = other DNA
aattagcttgactccttcccattaagacggaaaaaacatccgggagagccggtccgtttctcaggcagactaggccattaggtgcctcggagaaaggacccaaggctgctccgtccttcacagacacagtccaatcagagtttcccaggcacatcgatgcaccgcctccttcgagaaacaaggtaactttcgggttctggttgtctccaaagtcatccgaccaatctcgcaccgcccagagcgggcccttcctgtcaattacctactgaagggcaggcggccagcatcgccatggagaccaacacccttcccaccaccactccccctttctctcagggcccctgtcccctccagtgaatcccagaagactctggagagttctgagcagagggcggcaccctgccctctgattggtccaaggaaggctggggggcaggacgggaggcgaaacccctggaatattcccgacctggcagcctcatcgagcttggtgattggctcagaaggggaaaggcgggtctccacgacgacttataaaagccgaggggcgcgcggtccggaaaacggccagcctgaggagctgctgcgagggtccgcttcgtctttcgagagtgactcccgcggtcccaaggctttccagagcgaacctgtgcggctgcaggcaccggcgtgttgagtttccggcgttccgaaggactgagctcttgtcgcggatcccgtccgccgtttccagcccccagtctcagagcggagcccacagagcagggcaccggc

SEQ ID NO: 10 - DNA, 1060 bp, synthetic construct
  source 1..1060: mol_type = other DNA
aattagcttgaaaatcagtcaaacctaagaaaattctcaaccgcatcaaaccgaggaccaactgggacacagagcttctgccccactccaatcagagccttcccagctcacctgggatctctacgccttcgatccagtttggaaaatttcaagtcgctgagcccctacgagaggagctccaggaacataccaaactgaggcagccggggtcccccccaccccccaccccgcccctcccggcaactttgagcctgtgctgggacagagcctctagttcctaaattagtccatgaggtcagaggcagcactgccattgtaaccgcgattggagaggatcacgtcaccggacacgccccaggcatctccctgggtctcctaaacttggccggggagaagttttagcccttaaggttttagcctttaacccccatattcagaactgtgcgagttggcgaaaccccacaaatcacaacaaactgtacacaacaccgaggctagaggtgatctttcttgtccattccacacaggccttagtaattgcgtcgccatagcaacagtgtcactagtagcaccagcacgttccccacaccctccccctcaggaatccgtactctccagtgaaccccagaaacctctggagagttctggacaagggcggaacccacaactccgattactcaagggaggcggggaagctccaccagacgcgaaactgctggaagattcctggccccaaggcctcctccggctcgctgattggcccagcggagagtgggcggggccggtgaagactccttaaaggcgcagggcggcgagcacggtcaccagacgctgacagctactcagaaccaaatctggttccatccagagacaagcgaagacaagagaagcagagcagagcggcgcgttcccgatcctcggccaggaccagccttccccagagcatccctgccgcgggacgcaaccttcccaggagcatccctgccgcggagcaactttccccggagcatccagcccggacgcagccttccagaagcacgagcccaccactagtgccacc

SEQ ID NO: 11 - DNA, 1064 bp, synthetic construct
  source 1..1064: mol_type = other DNA
aattagcttgacctgcagcctgaggcaaagggagtggctacagcctggcacggtcgattaagccctgctctccgggtcctgggacactttcctttttcctcttttgagtcacaggtcctcctaacatgagaatcaagtattttcacgctgatttccttataaaattgtgagaactccataggcgatgtaccgcctactcctaccttaaccgtgatgtaaagacagcaaaacaaatgaactatactgcaagatctcttctatttccctattcaaacctaaaatgaagagggagggggagacatggacaagcaagcattccacaggcgcccctgcccaacgctgtcactcaaaccaggacccaatcacagactttttagccaagccttatcccgcctctcttgagaaactttctgcgtccgccatcctgtaggaaggatttgtacactttaaactccctccctggtctgagtcccacactctcaccacccagcaccttcaggagctgacccttaacagcttcacccacagggaccccgaagttgcgtcgcctccgcaacagtgtcaatagcagcaccagcacttccccacaccctccccctcaggaatccgtactctctagcgaaccccagaaacctctggagagttctggacaagggcggaacccacaactccgattactcaagggaggcggggaagctccaccagacgcgaaactgctggaagattcctggccccaaggcctcctccggctcgctgattggcccagcggagagtgggcggggccggtgaagactccttaaaggcgcagggcggcgagcagggcaccagacgctgacagctactcagaatcaaatctggttccatccagagacaagcgaagacaagagaagcagagcgagcggcgcgttcccgatcctcggccaggaccagccttccccagagcatccacgccgcggagcgcaaccttcccaggagcatccctgccgcggagcgcaactttccccggagcatccacgccgcggagcgcagccttccagaagcagagcgccaccactagtgccacc

SEQ ID NO: 12 - DNA, 231 bp, synthetic construct
  source 1..231: mol_type = other DNA
accagacgctgacagctactcagaaccaaatctggttccatccagagacaagcgaagacaagagaagcagagcgagcggcgcgttcccgatcctcggccaggaccagccttccccagagcatccctgccgcggagcgcaaccttcccaggagcatccctgccgcggagcgcaactttccccggagcatccacgccgcggagcgcagccttccagaagcagagcgcggcgcc

SEQ ID NO: 13 - DNA, 228 bp, synthetic construct
  source 1..228: mol_type = other DNA
gattgtagtttgaagatttcacaattagagtgaatgttgtttggttcggttttgtcactgtatttatactcatttccacctttttctagaaggtcctagatgcatctaggaccttctagaacattctaaacggctgcaggatacgggtatataagccaatcgtgttcagaggaaaccaatacactttgttcaagtgcttactgttcattctctaaacttcaagacacc

SEQ ID NO: 14 - DNA, 217 bp, synthetic construct
  source 1..217: mol_type = other DNA
acatttcctgtacaagtgccctaggagctcggatccaggaggcctaactggccggtacctgagctcctggaagattctagaacgttctggaagattctagaacgttcctcgaggatatcaagatctggcctcggcggccaagcttagacactagagggtatataatggaagctcgacttccagcttggcaatccggtactgttggtaaagcgccacc